SEQUENCE LISTING (92 sequences)

SEQ ID NO: 1 - protein, 139 aa, Synthetic Construct
MGSSHHHHHHSSGLVPRGSRGVPHIVMVDAYKRYKGSGESGMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGC

SEQ ID NO: 2 - protein, 139 aa, Synthetic Construct
MGSSHHHHHHSSGLVPRGSRGVPHIVMVAAYKRYKGSGESGMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGC

SEQ ID NO: 3 - protein, 302 aa, Synthetic Construct
MGSSHHHHHHSSGLVPRGSRGVPHIVMVDAYKRYKGSGESGYPYDVPDYASSGGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 4 - protein, 258 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGGYPYDVPDYAGGSC

SEQ ID NO: 5 - protein, 258 aa, Synthetic Construct; identical to SEQ ID NO: 4

SEQ ID NO: 6 - protein, 292 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLNGGYPYDVPDYAGGSC

SEQ ID NO: 7 - protein, 21 aa, Synthetic Construct
SGGENLYFQGENLYFQGGSGG

SEQ ID NO: 8 - protein, 581 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSACLSYETEILTVEYGLLPIGKIVEKRIECTVYSVDNNGNIYTQPVAQWHDRGKQKVFEYCLEDGSLIRATKDHKFMTVDGQMLPIKEIFRRKLDLMRVDNLPNGSGGENLYFQGENLYFQGGSGGIEIATEKYLGEQNVYDIGVERDHNFALKNGGYFQGIEIATEKYLGEQNVYDIGVERDHNFALKNGNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 9 - protein, 599 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGACLSYETEILTVEYGLLPIGKIVEKRIECTVYSVDNNGNIYTQPVAQWHDRGKQKVFEYCLEDGSLIRATKDHKFMTVDGQMLPIKEIFRRKLDLMRVDNLPNGSGGENLYFQGENLYFQGGSGGIEIATEKYLGEQNVYDIGVERDHNFALKNGGYFQGIEIATEKYLGEQNVYDIGVERDHNFALKNGNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 10 - protein, 612 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISCLSYETEILTVEYGLLPIGKIVEKRIECTVYSVDNNGNIYTQPVAQWHDRGKQKVFEYCLEDGSLIRATKDHKFMTVDGQMLPIKEIFRRKLDLMRVDNLPNGSGGENLYFQGENLYFQGGSGGIEIATEKYLGEQNVYDIGVERDHNFALKNGGYFQGIEIATEKYLGEQNVYDIGVERDHNFALKNGNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 11 - protein, 630 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLSYETEILTVEYGLLPIGKIVEKRIECTVYSVDNNGNIYTQPVAQWHDRGKQKVFEYCLEDGSLIRATKDHKFMTVDGQMLPIKEIFRRKLDLMRVDNLPNGSGGENLYFQGENLYFQGGSGGIEIATEKYLGEQNVYDIGVERDHNFALKNGGYFQGIEIATEKYLGEQNVYDIGVERDHNFALKNGNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 12 - protein, 638 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYTFVETAAPCLSYETEILTVEYGLLPIGKIVEKRIECTVYSVDNNGNIYTQPVAQWHDRGKQKVFEYCLEDGSLIRATKDHKFMTVDGQMLPIKEIFRRKLDLMRVDNLPNGSGGENLYFQGENLYFQGGSGGIEIATEKYLGEQNVYDIGVERDHNFALKNGGYFQGIEIATEKYLGEQNVYDIGVERDHNFALKNGNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 13 - protein, 647 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYTFVETAAPDGYEVATPICLSYETEILTVEYGLLPIGKIVEKRIECTVYSVDNNGNIYTQPVAQWHDRGKQKVFEYCLEDGSLIRATKDHKFMTVDGQMLPIKEIFRRKLDLMRVDNLPNGSGGENLYFQGENLYFQGGSGGIEIATEKYLGEQNVYDIGVERDHNFALKNGGYFQGIEIATEKYLGEQNVYDIGVERDHNFALKNGNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 14 - protein, 590 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGGEQEVFEYCLEDGSLIRATKDHKFMTVDGQMLPIDEIFERELDLMRVDNLPNGSGGENLYFQGENLYFQGGSGGIKIATRKYLGKQNVYDIGVERDHNFALKNGFIASNCHIKFSKRDEDGRELAGATMELRDSSGKTISTWISDGHVKDFYLYPGKYTFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 15 - protein, 572 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGGEQEVFEYCLEDGSLIRATKDHKFMTVDGQMLPIDEIFERELDLMRVDNLPNGSGGENLYFQGENLYFQGGSGGIKIATRKYLGKQNVYDIGVERDHNFALKNGFIASNCMELRDSSGKTISTWISDGHVKDFYLYPGKYTFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 16 - protein, 559 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGGEQEVFEYCLEDGSLIRATKDHKFMTVDGQMLPIDEIFERELDLMRVDNLPNGSGGENLYFQGENLYFQGGSGGIKIATRKYLGKQNVYDIGVERDHNFALKNGFIASNCWISDGHVKDFYLYPGKYTFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 17 - protein, 541 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGGEQEVFEYCLEDGSLIRATKDHKFMTVDGQMLPIDEIFERELDLMRVDNLPNGSGGENLYFQGENLYFQGGSGGIKIATRKYLGKQNVYDIGVERDHNFALKNGFIASNCFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 18 - protein, 532 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGGEQEVFEYCLEDGSLIRATKDHKFMTVDGQMLPIDEIFERELDLMRVDNLPNGSGGENLYFQGENLYFQGGSGGIKIATRKYLGKQNVYDIGVERDHNFALKNGFIASNCYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 19 - protein, 524 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGGEQEVFEYCLEDGSLIRATKDHKFMTVDGQMLPIDEIFERELDLMRVDNLPNGSGGENLYFQGENLYFQGGSGGIKIATRKYLGKQNVYDIGVERDHNFALKNGFIASNCFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 20 - protein, 595 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAIENLYGDEVDGYFQGMEAKTYIGKLKSRKIVSNEDTYDIQTSTHNFFANDNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 21 - protein, 595 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYALVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVDGYFQGMEAKTYIGKLKSRKIVSNEDTYDIQTSTHNFFANDNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 22 - protein, 595 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVDGYFQGMEAKTYIGKLKSRKIVSNEDTYDIQTSTHNFFANDNSAGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 23 - protein, 535 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGNAELVELEIDTLHGVKTIRCTPDHPVYTKNRGYVRADELTDDDELVVAIENLYGDEVDGYFQGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 24 - protein, 535 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGRVAILWHEMWHEGLEEASRLYFGERNVKGMFEVLEPLHAMMERGPQTLKETSFNQAYGRDLMEAQEWCRKYMKSGNVKDLLQAWDLYYHVFRRISGNGNGNAELVELEIDTLHGVKTIRVTPDHPVYTKNRGYVRADELTDDDELVVAIENLYGDEVDGYFQGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 25 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVDGYFQGMEAKTYIGKLKSRKIVSNEDTYDIQTSTHNFFAND

SEQ ID NO: 26 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFAND

SEQ ID NO: 27 - protein, 518 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFAND

SEQ ID NO: 28 - protein, 518 aa, Synthetic Construct; identical to SEQ ID NO: 27

SEQ ID NO: 29 - protein, 434 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGNAELVELEIDTLHGVKTIRVTPDHPVYTKNRGYVRADELTDDDELVVAIENLYGDEVDGYFQGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 30 - protein, 434 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGNAELVELEIKTLHGVKTIRVTPDHPVYTKNRGYVRADELTDDDELVVAIENLYGDEVNGYFQGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 31 - protein, 335 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGNAELVELEIKTLHGVKTIRVTPDHPVYTKNRGYVRADELTDDDELVVAIENLYGDEVNGYFQGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILMANGADVNAKDEYGLTPLYLATAHGHLEIVEVLLKNGADVNAVDAI

SEQ ID NO: 32 - protein, 436 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGNAELVELEIKTLHGVKTIRVTPDHPVYTKNRGYVRADELTDDDELVVAIENLYGDEVNGYFQGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLNEQKLISEEDL

SEQ ID NO: 33 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGALKSRAIVSNEDTYDIQTSTHNFFAND

SEQ ID NO: 34 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLASRAIVSNEDTYDIQTSTHNFFAND

SEQ ID NO: 35 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSAAIVSNEDTYDIQTSTHNFFAND

SEQ ID NO: 36 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGALASRAIVSNEDTYDIQTSTHNFFAND

SEQ ID NO: 37 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGALASAAIVSNEDTYDIQTSTHNFFAND

SEQ ID NO: 38 - protein, 484 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRKIVSNEDTYDIQTSTHNFFAND

SEQ ID NO: 39 - protein, 518 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGALASRAIVSNEDTYDIQTSTHNFFAND

SEQ ID NO: 40 - protein, 518 aa, Synthetic Construct; identical to SEQ ID NO: 39

SEQ ID NO: 41 - protein, 488 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFANDILVH

SEQ ID NO: 42 - protein, 487 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFANDILV

SEQ ID NO: 43 - protein, 486 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFANDIL

SEQ ID NO: 44 - protein, 485 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFANDI

SEQ ID NO: 45 - protein, 482 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFA

SEQ ID NO: 46 - protein, 481 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFF

SEQ ID NO: 47 - protein, 480 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNF

SEQ ID NO: 48 - protein, 479 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHN

SEQ ID NO: 49 - protein, 478 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTH

SEQ ID NO: 50 - protein, 475 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQT

SEQ ID NO: 51 - protein, 469 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNED

SEQ ID NO: 52 - protein, 449 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNGSTSGSVTTLSGLSGEQGPSGDMTTEEDSATHIKFSKRDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQG

SEQ ID NO: 53 - protein, 371 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGMEAKTYIGKLKSRKIVSNEDTYDIQTSTHNFFANDILVHNSFVETAAPDGYEVATPIEFTVNEDGQVTVDGEATEGDAHTGSTSGSDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNEQKLISEEDL

SEQ ID NO: 54 - protein, 510 aa, Synthetic Construct
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDYKDDDDKDLGKKLLEAARAGQDDEVRILMANGADVNADDTWGWTPLHLAAYQGHLEIVEVLLKNGADVNAYDYIGWTPLHLAADGHLEIVEVLLKNGADVNASDYIGDTPLHLAAHNGHLEIVEVLLKHGADVNAQDKFGKTAFDISIDNGNEDLAEILQKLNGSTSGSVTTLSGLSGEQGPSGDMTTEEDSATHIKFSERDEDGRELAGATMELRDSSGKTISTWISDGHVKDFYLYPGKYCLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGALASRAIVSNEDTYDIQTSTH

SEQ ID NO: 55 - protein, 113 aa, Synthetic Construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 56 - protein, 6 aa, Synthetic Construct
VELEID

SEQ ID NO: 57 - protein, 6 aa, Synthetic Construct
KRSKLK

SEQ ID NO: 58 - protein, 9 aa, Synthetic Construct
ADELTDDDE

SEQ ID NO: 59 - protein, 17 aa, Synthetic Construct
MEAKTYIGKLKSRKIVS

SEQ ID NO: 60 - protein, 17 aa, Synthetic Construct
MEAETYIGKLKSRAIVS

SEQ ID NO: 61 - protein, 17 aa, Synthetic Construct
MEAETYIGALKSRAIVS

SEQ ID NO: 62 - protein, 17 aa, Synthetic Construct
MEAETYIGKLASRAIVS

SEQ ID NO: 63 - protein, 17 aa, Synthetic Construct
MEAETYIGKLKSAAIVS

SEQ ID NO: 64 - protein, 17 aa, Synthetic Construct
MEAETYIGALASRAIVS

SEQ ID NO: 65 - protein, 17 aa, Synthetic Construct
MEAETYIGALASAAIVS

SEQ ID NO: 66 - protein, 17 aa, Synthetic Construct
MEAETYIGKLKSRKIVS

SEQ ID NO: 67 - protein, 7 aa, Synthetic Construct
KAAQLTR

SEQ ID NO: 68 - protein, 20 aa, Synthetic Construct
TYDIQTSTHNFFANDILVHN

SEQ ID NO: 69 - protein, 6 aa, Synthetic Construct
IVSNED

SEQ ID NO: 70 - protein, 12 aa, Synthetic Construct
IVSNEDTYDIQT

SEQ ID NO: 71 - protein, 15 aa, Synthetic Construct
IVSNEDTYDIQTSTH

SEQ ID NO: 72 - protein, 16 aa, Synthetic Construct
IVSNEDTYDIQTSTHN

SEQ ID NO: 73 - protein, 17 aa, Synthetic Construct
IVSNEDTYDIQTSTHNF

SEQ ID NO: 74 - protein, 17 aa, Synthetic Construct
VSNEDTYDIQTSTHNFF

SEQ ID NO: 75 - protein, 18 aa, Synthetic Construct
VSNEDTYDIQTSTHNFFA

SEQ ID NO: 76 - protein, 20 aa, Synthetic Construct
IVSNEDTYDIQTSTHNFFAN

SEQ ID NO: 77 - protein, 21 aa, Synthetic Construct
IVSNEDTYDIQTSTHNFFAND

SEQ ID NO: 78 - protein, 22 aa, Synthetic Construct
IVSNEDTYDIQTSTHNFFANDI

SEQ ID NO: 79 - protein, 23 aa, Synthetic Construct
IVSNEDTYDIQTSTHNFFANDIL

SEQ ID NO: 80 - protein, 24 aa, Synthetic Construct
IVSNEDTYDIQTSTHNFFANDILV

SEQ ID NO: 81 - protein, 25 aa, Synthetic Construct
IVSNEDTYDIQTSTHNFFANDILVH

SEQ ID NO: 82 - protein, 113 aa, Synthetic Construct; identical to SEQ ID NO: 55

SEQ ID NO: 83 - protein, 154 aa, Synthetic Construct
CLVGSSEIITRNYGKTTIKEVVEIFDNDKNIQVLAFNTHTDNIEWAPIKAAQLTRPNAELVELEIDTLHGVKTIRVTPDHPVYTKNRGYVRADELTDDDELVVAIENLYGDEVNGYFQGMEAETYIGKLKSRAIVSNEDTYDIQTSTHNFFAND

SEQ ID NO: 84 - protein, 103 aa, Synthetic Construct
NAELVELEIKTLHGVKTIRVTPDHPVYTKNRGYVRADELTDDDELVVAIENLYGDEVNGYFQGMEAKTYIGKLKSRKIVSNEDTYDIQTSTHNFFANDILVHN

SEQ ID NO: 85 - protein, 154 aa, Synthetic Construct
CLVGSSEIITRNYGKTTIKEVVEIFDNDKNIQVLAFNTHTDNIEWAPIKAAQLTRPNAELVELEIDTLHGVKTIRCTPDHPVYTKNRGYVRADELTDDDELVVAIENLYGDEVDGYFQGMEAKTYIGKLKSRKIVSNEDTYDIQTSTHNFFAND

SEQ ID NO: 86 - protein, 103 aa, Synthetic Construct
NAELVELEIDTLHGVKTIRCTPDHPVYTKNRGYVRADELTDDDELVVAIENLYGDEVDGYFQGMEAKTYIGKLKSRKIVSNEDTYDIQTSTHNFFANDILVHN

SEQ ID NO: 87 - protein, 181 aa, Synthetic Construct
CLSYETKILTVEYGLLPIGKIVEKRIECTVYSVDNNGNIYTQPVAQWHDRGKQKVFEYCLKDGSLIRATKDHKFMTVDGQMLPIKEIFRRKLDLMRVDNLPNENLYGDEVDGYFQGIEIATEKYLGEQNVYDIGVERDHNFALENGGYFQGIEIATEKYLGEQNVYDIGVERDHNFALENG

SEQ ID NO: 88 - protein, 101 aa, Synthetic Construct
GEQEVFEYCLEDGSLIRATKDHKFMTVDGQMLPIDEIFERELDLMRVDNLPNENLYGDEVDGYFQGIKIATRKYLGKQNVYDIGVERDHNFALKNGFIASN

SEQ ID NO: 89 - protein, 134 aa, Synthetic Construct
CLDLKTQVQTPQGMKEISNIQVGDLVLSNTGYNEVLNVFPKSKKKSYKITLEDGKEIICSEEHLFPTQTGEMNISGGLKEGMCLYVKEGNLYGDEVDGYFQGMMLKKILKIEELDERELIDIEVSGNHLFYAND

SEQ ID NO: 90 - protein, 98 aa, Synthetic Construct
SKKKSYKITLEDGKEIICSEEHLFPTQTGEMNISGGLKEGMCLYVKEGNLYGDEVDGYFQGMMLKKILKIEELDERELIDIEVSGNHLFYANDILTHN

SEQ ID NO: 91 - protein, 143 aa, Synthetic Construct
CLSLDTMVVTNGKAIEIRDVKVGDWLESECGPVQVTEVLPIIKQPVFEIVLKSGKKIRVSANHKFPTKDGLKTINSGLKVGDFLRSRAKENLYGDEVDGYFQGMCEIFENEIDWDEIASIEYVGVEETIDINVTNDRLFFANG

SEQ ID NO: 92 - protein, 108 aa, Synthetic Construct
IIKQPVFEIVLKSGKKIRVSANHKFPTKDGLKTINSGLKVGDFLRSRAKENLYGDEVDGYFQGMCEIFENEIDWDEIASIEYVGVEETIDINVTNDRLFFANGILTHN

(19) United States
(12) Patent Application Publication
(10) Pub. No.: US 2026/0053882 A1
(43) Pub. Date: Feb. 26, 2026

(54) LOGIC GATED PROTEIN ACTUATORS

(71) Applicant: THE TRUSTEES OF PRINCETON UNIVERSITY, Princeton, NJ (US)

(72) Inventors: Thomas W. MUIR, Princeton, NJ (US); Christian KOFOED, Copenhagen (DK); Gihoon LEE, Princeton, NJ (US); Josef GRAMESPACHER, Princeton, NJ (US); Nicholas TAY, Princeton, NJ (US); Xuanjia YE, Princeton, NJ (US); Girum ERKALO, Princeton, NJ (US)

(73) Assignee: THE TRUSTEES OF PRINCETON UNIVERSITY, Princeton, NJ (US)

(21) Appl. No.: 19/105,551

(22) PCT Filed: Aug. 23, 2023
(86) PCT No.: PCT/US2023/072702

§ 371 Date: Feb. 21, 2025

Related U.S. Application Data
(60) Provisional application No. 63/400,218, filed on Aug. 23, 2022.

(51) Int. Cl. A61K 38/00; A61K 49/00; A61P 35/00; C07K 14/71; C07K 16/28; C07K 16/40; C12N 9/00

(52) U.S. Cl. CPC A61K 38/00; A61K 49/0002; A61P 35/00; C07K 14/71; C07K 16/2809; C07K 16/40; C12N 9/00; C07K 2319/70

(57) ABSTRACT

A logic-gated protein device in which proximity-gated protein trans-splicing governs formation of an active protein from two otherwise inactive fragments.

This International Application claims the benefit of U.S. Provisional Application No. 63/400,218, filed Aug. 23, 2022, the specification of which is hereby incorporated by reference in its entirety.

This invention was made with government support under Grant Nos. R37-GM096868 and R01-GM086868 awarded by the National Institutes of Health. The government has certain rights in the invention.

### FIELD OF THE INVENTION

The present invention relates to logic gated protein actuators.

### SUMMARY OF THE INVENTION

In an embodiment of the invention, a logic-gated protein actuator system includes a mixture that includes a first component and a second component. The first component can include a first caged split intein fragment fused to a first protein-of-interest fragment and fused to a first antigen-binding domain configured to bind to a first surface antigen. The second component can include a second caged split intein fragment fused to a second protein-of-interest fragment and fused to a second antigen-binding domain configured to bind to a second surface antigen. The first caged split intein fragment and the second caged split intein fragment can be capable of undergoing protein trans-splicing to form a protein of interest from the first protein-of-interest fragment and the second protein-of-interest fragment when the first antigen-binding domain and the second antigen-binding domain are proximal (e.g., near to each other, next to each other, or in contact with each other). The first caged split intein fragment and the second caged split intein fragment can be considered to be an actuator. The first surface antigen and the second surface antigen can be different.

In an embodiment of the invention, the mixture does not include a third component including the first caged split intein fragment fused to the first protein-of-interest fragment and fused to the second antigen-binding domain, and the mixture does not include a fourth component comprising the second caged split intein fragment fused to the second protein-of-interest fragment and fused to the first antigen-binding domain; this logic-gated protein actuator system is a first-surface-antigen-AND-second-surface-antigen logic gate.

In an embodiment of the invention, the mixture includes a fifth component includes the second caged split intein fragment fused to the second protein-of-interest fragment and fused to a third antigen-binding domain configured to bind to a third surface antigen. The third surface antigen is different from the first surface antigen and from the second surface antigen. The first caged split intein fragment and the second caged split intein fragment can undergo protein trans-splicing to form the protein of interest from the first protein-of-interest fragment and the second protein-of-interest fragment when the third antigen-binding domain and the first antigen-binding domain are proximal. This logic-gated protein actuator system is a first-surface-antigen-AND-either-second-surface-antigen-OR-third-surface-antigen logic gate.

The mixture can include at least one additional component; each additional component includes the second caged split intein fragment fused to the second protein-of-interest fragment and fused to an additional antigen-binding domain configured to bind to an additional surface antigen. Each additional surface antigen of each additional component can be different from the first surface antigen, the second surface antigen, the third surface antigen, and each other additional surface antigen. The first caged split intein fragment and the second caged split intein fragment can undergo protein trans-splicing to form the protein of interest from the first protein-of-interest fragment and the second protein-of-interest fragment when the additional antigen-binding domain is proximal to the third antigen-binding domain, the second antigen-binding domain, the first antigen-binding domain, or any other additional binding domain. This logic-gated protein actuator system can be a first-surface-antigen-AND-either-second-surface-antigen-OR-third-surface-antigen-OR-additional-surface-antigen(s) logic gate.

In an embodiment of the invention, a logic-gated protein actuator system includes a fifth component including the second caged split intein fragment fused to a modified second protein-of-interest fragment and fused to a third antigen-binding domain configured to bind to a third surface antigen. The third surface antigen is different from the first surface antigen and from the second surface antigen. The first caged split intein fragment and the second caged split intein fragment of the fifth component can undergo protein trans-splicing to form a defective protein of interest from the first protein-of-interest fragment and the modified second protein-of-interest fragment when the third antigen-binding domain and the first antigen-binding domain are proximal. The defective protein of interest does not produce an effect of the protein of interest, such as binding a toxin or an imaging agent. This logic-gated protein actuator system is a first-surface-antigen-AND-second-surface-antigen-NOT-third-surface-antigen logic gate.

The first surface antigen and the second surface antigen can be identical. The logic-gated protein actuator system can be a first-surface-antigen-NOT-third-surface-antigen logic gate.

The mixture can include a sixth component including the second caged split intein fragment fused to the modified second protein-of-interest fragment and fused to a fourth antigen-binding domain configured to bind to a fourth surface antigen. The fourth surface antigen can be different from the first surface antigen, from the second surface antigen, and from the third surface antigen. The first caged split intein fragment and the second caged split intein fragment of the sixth component can undergo protein trans-splicing to form the defective protein of interest from the first protein-of-interest fragment and the modified second protein-of-interest fragment when the fourth antigen-binding domain and the first antigen-binding domain are proximal. This logic-gated protein actuator system can be a first-surface-antigen-AND-second-surface-antigen-NOT-third-surface-antigen-NOT-fourth-surface-antigen logic gate.

The first surface antigen and the second surface antigen can be identical. The logic-gated protein actuator system can be a first-surface-antigen-AND-[third-surface-antigen-NOR-fourth-surface-antigen] logic gate.

The mixture can include at least one additional component including the second caged split intein fragment fused to the modified second protein-of-interest fragment and fused to an additional antigen-binding domain configured to bind to an additional surface antigen. The additional surface antigen can be different from the first surface antigen, from the second surface antigen, from the third surface antigen, and from the fourth surface antigen. The first caged split intein fragment and the second caged split intein fragment of the additional component can undergo protein trans-splicing to form the defective protein of interest from the first protein-of-interest fragment and the modified second protein-of-interest fragment when the additional antigen-binding domain and the first antigen-binding domain are proximal. This logic-gated protein actuator system can be a first-surface-antigen-AND-second-surface-antigen-NOT-third-surface-antigen-NOT-fourth-surface-antigen-NOT-additional-surface-antigen logic gate.

In an embodiment of the invention, a logic-gated protein actuator system the first surface antigen and the second surface antigen are different from each other. The mixture includes a third component including the first caged split intein fragment fused to the first protein-of-interest fragment and fused to the second antigen-binding domain, and the mixture includes a fourth component including the second caged split intein fragment fused to the second protein-of-interest fragment and fused to the first antigen-binding domain. The first caged split intein fragment and the second caged split intein fragment can undergo protein trans-splicing to reconstitute the protein of interest from the first protein-of-interest fragment and the second protein-of-interest fragment when two first antigen-binding domains are proximal. The first caged split intein fragment and the second caged split intein fragment can undergo protein trans-splicing to reconstitute the protein of interest from the first protein-of-interest fragment and the second protein-of-interest fragment when two second antigen-binding domains are proximal This logic-gated protein actuator system is a first-surface-antigen-OR-second-surface-antigen logic gate.

The mixture can include a fifth component including the first caged split intein fragment fused to the first protein-of-interest fragment and fused to a third antigen-binding domain, and the mixture can include a sixth component including the second caged split intein fragment fused to the second protein-of-interest fragment and fused to a fourth antigen-binding domain. The third antigen-binding domain can be different from the second antigen-binding domain and from the first antigen-binding domain. The first caged split intein fragment and the second caged split intein fragment can undergo protein trans-splicing to reconstitute the protein of interest from the first protein-of-interest fragment and the second protein-of-interest fragment when the third antigen-binding domain is proximal to the first antigen-binding domain or the second antigen-binding domain or when two third antigen-binding domains are proximal. The logic-gated protein actuator system can be a first-surface-antigen-OR-second-surface-antigen-OR-third-surface-antigen logic gate.

The mixture can include at least one additional pair of components. The first part of the additional pair of components can include the first caged split intein fragment fused to the first protein-of-interest fragment and fused to an additional antigen-binding domain configured to bind to an additional surface antigen. The second part of the additional pair of components can include the second caged split intein fragment fused to the second protein-of-interest fragment and fused to the additional antigen-binding domain. Each additional surface antigen can be different from the first surface antigen, the second surface antigen, the third surface antigen, and each other additional surface antigen. The first caged split intein fragment and the second caged split intein fragment can undergo protein trans-splicing to form the protein of interest from the first protein-of-interest fragment and the second protein-of-interest fragment when the additional antigen-binding domain is proximal to the third antigen-binding domain, the second antigen-binding domain, the first antigen-binding domain, or any additional binding domain. The logic-gated protein actuator system can be a first-surface-antigen-OR-second-surface-antigen-OR-third-surface-antigen-OR-additional-surface-antigen(s) logic gate.

In a logic-gated protein actuator system, the first caged split intein fragment can be eNrdJ-1N^{cage}-K114AK116A and the second caged split intein fragment can be eNrdJ-1C^{cage}. The first caged split intein fragment can be eNrdJ-1C^{cage} and the second caged split intein fragment can be eNrdJ-1N^{cage}-K114AK116A. The pair of the first caged split intein fragment and the second caged split intein fragment can be eNrdJ-1N^{cage} & eNrdJ-1C^{cage}, NrdJ-1N^{cage} & NrdJ-1C^{cage}, NpuN^{cage} & NpuC^{cage}, Gp41-1N^{cage} & Gp41-1C^{cage}, Gp41-8N^{cage} & Gp41-8C^{cage}, eNrdJ-1C^{cage} & eNrdJ-1N^{cage}, NrdJ-1C^{cage} & NrdJ-1N^{cage}, NpuC^{cage} & NpuN^{cage}, Gp41-1C^{cage} & Gp41-1N^{cage}, or Gp41-8C^{cage} & Gp41-8N^{cage}. For example, the protein of interest can include a toxin, an enzyme, an imaging agent, and/or an antibody epitope, an antigen, and/or or an antigen fragment to which an antibody and/or an antibody paratope and/or a T-cell and/or a T-cell receptor can bind, and/or a protein, a protein fragment, an antigen, an antibody, and/or an antigen-antibody complex to which a complement protein can bind. For example, the protein of interest can bind a toxin, an enzyme, an imaging agent, and/or an antibody epitope, an antigen, and/or an antigen fragment to which an antibody and/or an antibody paratope and/or a T-cell and/or a T-cell receptor can bind, and/or a protein, a protein fragment, an antigen, an antibody, and/or an antigen-antibody complex to which a complement protein can bind.

In a logic-gated protein actuator system, the protein of interest can be SpyCatcher003. The first protein of interest fragment can be SpyN¹⁻⁷³, and the second protein of interest fragment can be SpyC⁷⁴⁻¹¹³. The first protein of interest fragment can be SpyC⁷⁴⁻¹¹³, and the second protein of interest fragment can be SpyN¹⁻⁷³. The pair of the first protein-of-interest fragment and the second protein-of-interest fragment can be SpyN¹⁻²⁴ & SpyC²⁵⁻¹¹³, SpyN¹⁻⁴² & SpyC⁴³⁻¹¹³, SpyN¹⁻⁵⁵ & SpyC⁵⁶⁻¹¹³, SpyN¹⁻⁸² & SpyC⁸³⁻¹¹³, SpyN¹⁻⁹⁰ & SpyC⁹¹⁻¹¹³, SpyC²⁵⁻¹¹³ & SpyN¹⁻²⁴, SpyC⁴³⁻¹¹³ & SpyN¹⁻⁴². SpyC⁵⁶⁻¹¹³ & SpyN¹⁻⁵⁵, SpyC⁸³⁻¹¹³ & SpyN¹⁻⁸², or SpyC⁹¹⁻¹¹³ & SpyN^{1-90.}

In a logic-gated protein actuator system, the mixture can include a SpyTag003-biotin conjugate. The mixture can include a SpyTag003-biotin conjugate and a Streptavidin-Saporin conjugate. The mixture can include a SpyTag003-AF594 conjugate. The mixture can include a SpyTag003-APEX2 conjugate.

In a logic-gated protein actuator system, the first surface antigen can be HER2, EGFR, or EpCAM. The second surface antigen can be HER2, EGFR, or EpCAM. The third surface antigen can be HER2, EGFR, or EpCAM. Each surface antigen can be HER2. EGFR, or EpCAM.

In a method according to the invention, a protein of interest is conditionally formed. A mixture is provided that includes a first component including a first caged split intein fragment fused to a first protein-of-interest fragment and fused to a first antigen-binding domain configured to bind to a first surface antigen, and that includes a second component including a second caged split intein fragment fused to a second protein-of-interest fragment and fused to a second antigen-binding domain configured to bind to a second surface antigen. The mixture is brought into contact with a cell having a surface; for example, the mixture is brought into contact with the surface of a cell. When the first surface antigen and the second surface antigen are proximal on the surface of the cell, first antigen-binding domain binds to the first surface antigen and the second antigen-binding domain binds to the second surface antigen. The first caged split intein fragment and the second caged split intein fragment undergo protein trans-splicing to form a protein of interest from the first protein-of-interest fragment and the second protein-of-interest fragment. The first caged split intein fragment and the second caged split intein fragment are an actuator.

The protein of interest can bind a toxin, and the toxin can damage or kill the cell. A cancer can be treated by administering the mixture and the toxin to a patient, the cell being a cancerous cell.

The protein of interest can bind an imaging agent, and the imaging agent can be used to image the cell. A hyperproliferative disorder can be diagnosed by administering the mixture and the imaging agent to a patient, the cell being a hyperproliferative cell.

The protein of interest can include a toxin, and the toxin can damage or kill the cell. The protein of interest can include an imaging agent, and the imaging agent can be used to image the cell.

### BRIEF DESCRIPTION OF THE DRAWINGS

FIG. 1A. Tissues and cell communities exhibit similarities and variations in surface antigens. Unique antigen combinations generate distinct surface profiles, which can be used for specific targeting by Boolean logic functions by implementing a protein switch that computes Boolean logic on live cell surfaces by using conditional protein splicing.

FIG. 1B. Protein trans-splicing is a spontaneous and masked AND gated reaction: Split intein fragments rapidly associate, fold, and react to reconstitute a protein of interest (POI).

FIG. 1C. While inactive in the dilute solution phase, the caged split intein undergoes conditional protein splicing when its two components colocalize and locally concentrate on a target cell surface thereby causing input-to-output actuation.

FIG. 1D. As a functional output SpyCatcher003 was split at position 73-74 and fused to NrdJ-1^{cage} thereby generating SMART-SpyCatcher. While the split version of SpyCatcher003 is inactive, the reconstituted form can covalently associate with SpyTag003 through the formation of an isopeptide bond.

FIG. 1E. SMART-SpyCatcher (SpyN-αHER2 and SpyC-αEGFR) targeted for [HER2 AND EGFR] logic was incubated with individual K562, K562^{HER2+}, K562^{EGFR+}, and K562^{HER2+/EGFR+} cell lines. SpyTag003 labeled with Alexa Fluor 594 (SpyTag003-AF594) was used to detect any actuation.

FIG. 1F. The ability of SMART-SpyCatcher to discriminate between cell lines through [HER2 AND EGFR] logic was further assayed on a mixed population of the four K562 cell lines described above.

FIG. 1G. Live cell flow cytometry of the mixed K562 population allowed for the analysis of each subpopulation post-assaying.

FIG. 1H. Dose-response experiment on the mixed K562 population with a dilution series of SMART-SpyCatcher (1 μM-1 nM) and excess SpyTag003-AF594. Unless otherwise noted, experiments were performed with 100 nM SMART-SpyCatcher (100 nM SpyN-αHER2 and 100 nM SpyC-αEGFR employing eNrdJ-1^{cage}), and 100 nM SpyTag003-AF594. Flow cytometry data are presented as the mean with error bars signifying the standard error mean (n=3 independent biological replicates).

FIG. 2A. Colocalization and activation of SMART-SpyCatcher can in principle occur from binding to two target antigens that are already closely associated or that will stochastically encounter each other on the cell membrane.

FIG. 2B. SMART-SpyCatcher (i.e., SpyN and SpyC), was assigned to operate through AND logic involving combinations of EpCAM with HER2 and EGFR. Two combinations of mixed K562 cell lines were used to test for the recruitment of SpyTag003-AF594 (mixed population 1 was K562^{EpCAMlo, K562EGFR+/EpCAMlo}, K562^{HER2+/EpCAMlo} and K562^{HER2+/EGFR+/EpCAMlo}; mixed population 2 was K562^{EpCAMlo}, K562^{EGFR+/EpCAMlo}, K562^{HER2+/EpCAMhi}, and K562^{HER2+/EGFR+/EpCAMhi}) and the data combined in the bar graph plot. Quantification of AF594 signal was done by flow cytometry. The antigen profile of each cell line is indicated below each bar plot.

FIG. 2C. Recruitment of SpyTag003-AF594 to K562^{HER2+} cell lines with low or high levels of EpCAM could be altered by employing tuned versions of eNrdJ-1N^{cage} in a [HER2 AND EpCAM] setting.

FIG. 2D. Cell targeting using [Ag1 AND either Ag2 OR Ag3] (3-input) logic results in broad yet cell-specific recruitment of SpyTag003-AF594.

FIG. 2E. SMART-SpyCatcher can navigate precisely between cell lines using 3-input AND/OR functions to target subpopulations specifically. Data were acquired as for FIG. 2B using the two described mixed K562 populations.

FIG. 2F. The SMART system can encompass NOT operators as exemplified in the [HER2 AND EpCAM NOT EGFR] setting. Here, the Decoy-αEGFR obstructs AND logic output on K562^{HER2+/EGFR+/EpCAMhi} since splicing between the Decoy and SpyC generates a defective output unable to recruit SpyTag003-AF594. In contrast K562^{HER2+/EpCAMhi} elicit a normal response as Decoy-αEGFR does not bind to the cell.

FIG. 2G. SMART-SpyCatcher was assigned for [HER2 AND EpCAM] (2-input) logic and tested on the mixed K562 population 2 described above. When Decoy-αEGFR was added to generate [HER2 AND EpCAM NOT EGFR] (3-input) logic, disruption of proper AND output was observed for K562^{HER2/EGFR/EpCAMhi+} cells with little-to-no effect on K562^{HER2/EpCAMhi}. All experiments were performed with 100 nM SMART-SpyCatcher and 100 nM SpyTag003-AF594. Data arc presented as the mean with error bars signifying the standard error mean (n=3 independent biological replicates).

FIG. 2H. Data shown are for the systems described for FIG. 2G. Relative MFI % is shown for the indicated concentration of Decoy-αEGFR.

FIG. 3A. SMART-SpyCatcher performs AND logic on cell lines with endogenous antigen profiles. Target cell lines were profiled for their relative surface levels of HER2, EGFR, and EpCAM using targeting DARPins labeled with AF594. In parallel, the AND logic matrix for SMART-SpyCatcher were solved for each cell line using SpyN/SpyC fused to combinations of the targeting DARPins. Phenotyping and SpyTag003-AF594 recruitment was quantified by flow cytometry.

FIG. 3B. The relative surface levels of HER2, EGFR, and EpCAM for the OE19 cell line (an esophageal cancer cell line) are shown.

FIG. 3C. The solved AND logic matrix for SMART-SpyCatcher using pairs of the three antigens to recruit SpyTag003-AF594 to OE19 is shown.

FIG. 3D. The collective data across all tested cancer cell lines were analyzed: The median quantity of the lesser-expressed antigen used in each AND gate is plotted against the achieved recruitment of SpyTag003-AF594 by the given logic operation. Where relevant, experiments were performed with 100 nM targeting DARPin-AF594, 100 nM SMART-SpyCatcher, and 100 nM SpyTag003-AF594.

FIG. 3E. The endogenous surface levels of HER2 and EpCAM on mammary cell lines MCF-10a, MCF-7, and SK-br-3 were profiled as in described for FIG. 3A. The two antigens were detected across all three cell lines and categorized as low (MFI<1,000) or high (MFI≥1,000).

FIG. 3F. The endogenous surface levels of HER2 and EpCAM on mammary cell lines MCF-10a, MCF-7, and SK-br-3 were profiled as in described for FIG. 3A. The two antigens were detected across all three cell lines and categorized as low (MFI<1,000) or high (MFI≥1,000).

FIG. 3G. SMART-SpyCatcher assigned for [HER2 AND EpCAM] was tested in mixed population flow cytometry experiments using mixed population 1 (MCF-10a^{HER2lo/EpCAMlo}/MCF-7^{HER2lo/EpCAMhi}) and mixed population 2 (MCF-10a^{HER2lo/EpCAMlo}/Sk-br-3^{HER2hi/EpCAMhi}). Each mixed population was incubated with 100 nM SpyTag003-AF594 in the absence or presence of 100 nM SpyN-αHER2 and SpyC-αEpCAM (employing eNrdJ-1^{cage} with K114AK116A). MCF-10a was pre-stained with CMFDA, which labels intracellular proteins. Shown arc representative flow cytometry plots of the recruitment of SpyTag003-AF594 under the different conditions by the two mixed populations.

FIG. 3H. Data for the system of FIG. 3G are presented as the mean with error bars signifying the standard error mean (n=3 independent biological replicates).

FIG. 4A. AND gated cell specific surface sculpting has various applications. Through AND gated splicing, SpyCatcher003 constitutes a loading dock that can allow for the highly-selective delivery of various cargoes of interest. For instance, delivery of SpyTag003-biotin allows for the recruitment of streptavidin with a toxic payload.

FIG. 4B. The initial recruitment of and surface decoration with Neutravidin Rhodamine Red-X conjugate leads to an intact protein complex over time through endocytosis.

FIG. 4C. A mixed K562 population (K562, K562^{HER2+}, K562^{EGFR+}, K562^{HER2+/EGFR+}) was treated with SMART-SpyCatcher implementing [HER2 AND EGFR] logic and SpyTag003-biotin. The selective recruitment of Neutravidin Rhodamine Red-X was monitored by flow cytometry.

FIG. 4D. Mixed K562 was treated as in described for FIG. 4C but using Streptavidin-Saporin (toxin) to deplete cells that induced SMART-SpyCatcher actuation.

FIG. 4E. The system of FIG. 4D resulted in the selective depletion of the K562^{HER2+/EGFR+} cell line with no detectable effect on the other subpopulations. Experiments were performed with 100 nM SpyN-αHER2, 100 nM SpyC-αEGFR, 100 nM SpyTag003-biotin and 20 nM Streptavidin-Saporin as specified. Results were obtained by flow cytometry.

FIG. 4F. Delivery of SpyTag003-APEX2 can allow for AND gated protein labeling with biotin-phenol.

FIG. 4G. Various K562 cell lines were tested for the [HER2 AND EGFR] gated APEX2 proximity protein labeling using 100 nM SpyN-αHER2,100 nM SpyC-αEGFR, and 300 nM SpyTag003-APEX2.

FIG. 4H. Overview of the three AND gates used in the experiments for which results are shown in FIGS. 4I and 4J.

FIG. 4I. Similar to FIG. 4G, using [HER2 AND EGFR], [HER2 AND EpCAM], and [EGFR AND EpCAM] logic operations, SMART-SpyCatcher allows for selective delivery of and subsequent proximity protein labeling by SpyTag003-APEX2. The three logic gates described in FIG. 4H were used on K562^{HER2+/EGFR+}, OE19, and A431.

FIG. 4J. Quantification of the APEX2 labeling results from FIG. 4I. All experiments were performed with 100 nM SMART-SpyCatcher and 300 nM SpyTag003-APEX2 when specified. Data are presented as the mean with error bars signifying the standard error mean (n=3 independent biological replicates).

FIG. 5. Conditional protein splicing of SpyN¹⁻²⁴-NpuNcage-FKBP and FRB-NpuC^{cage}-SpyC²⁵⁻¹¹³. A conditional protein splicing screen was used inducing splicing either by proteolytical decaging or through chemically induced dimerization. The use of FKB-FRB heterodimerization upon addition of rapamycin simulates perfect colocalization on a target cell surface. Here the split site 24-25 for SpyCatcher003 was tested, which showed background reactivity of FRB-NpuC^{cage}-SpyC²⁵⁻¹¹³ as it retains most of the active fold. SpyN¹⁻²⁴-NpuN^{cage}-FKBP and FRB-NpuC^{cage}-SpyC²⁵⁻¹¹³ were fused to a FLAG and Myc tag respectively; His₆-SpyTag003 was used as a probe. SpyCatcher003 alone or incubated with the probe was used as a positive control for splicing or SpyCatcher003-SpyTag003 reactivity, respectively.

FIG. 6. Conditional protein splicing of SpyN¹⁻⁴²-NpuNcage-FKBP and FRB-NpuC^{cage}-SpyC⁴³⁻¹¹³. A conditional protein splicing screen was used inducing splicing either by proteolytical decaging or through chemically induced dimerization. The use of FKB-FRB heterodimerization upon addition of rapamycin simulates perfect colocalization on a target cell surface. Here the split site 42-43 for SpyCatcher003 was tested, which generated a version that was inactive until spliced. SpyN¹⁻⁴²-NpuNcage-FKBP and FRB-NpuC^{cage}-SpyC⁴³⁻¹¹³ were fused to a FLAG and Myc tag respectively, His₆-SpyTag003 was used as a probe. SpyCatcher003 alone or incubated with the probe was used as a positive control for splicing or SpyCatcher003-SpyTag003 reactivity, respectively.

FIG. 7. Conditional protein splicing of SpyN¹⁻⁵⁵-NpuN^{cage}-FKBP and FRB-NpuC^{cage}-SpyC⁵⁶⁻¹¹³. A conditional protein splicing screen was used inducing splicing either by proteolytical decaging or through chemically induced dimerization. The use of FKB-FRB heterodimerization upon addition of rapamycin simulates perfect colocalization on a target cell surface. Here the split site 55-56 for SpyCatcher003 was tested, which showed low residual background reactivity of SpyN¹⁻⁵⁵-NpuN^{cage}-FRB when incubated with FRB-NpuC^{cage}-SpyC⁵⁶⁻¹¹³, presumably as the pair can refold without splicing. SpyN¹⁻⁵⁵-NpuN^{cage}-FKBP and FRB-NpuC^{cage}-SpyC⁵⁶⁻¹¹³ are fused to a FLAG and Myc tag respectively, His₆-SpyTag003 were used as a probe. SpyCatcher003 alone or incubated with the probe was used as a positive control for splicing or SpyCatcher003-SpyTag003 reactivity, respectively.

FIG. 8. Conditional protein splicing of SpyN¹⁻⁸²-NpuN^{cage}-FKBP and FRB-NpuC^{cage}-SpyC⁸³⁻¹¹³. A conditional protein splicing screen was used inducing splicing either by proteolytical decaging or through chemically induced dimerization. The use of FKB-FRB heterodimerization upon addition of rapamycin simulates perfect colocalization on a target cell surface. Here the split site 82-83 for SpyCatcher003 was tested, which generated a version that was inactive until spliced. SpyN¹⁻⁸²-NpuNcage-FKBP and FRB-NpuC^{cage}-SpyC⁸³⁻¹¹³ were fused to a FLAG and Myc tag respectively, His₆-SpyTag003 was used as a probe. SpyCatcher003 alone or incubated with the probe was used as a positive control for splicing or SpyCatcher003-SpyTag003 reactivity, respectively.

FIG. 9. Conditional protein splicing of SpyN¹⁻⁹⁰-NpuN^{cage}-FKBP and FRB-NpuC^{cage}-SpyC⁹¹⁻¹¹³. A conditional protein splicing screen was used inducing splicing either by proteolytical decaging or through chemically induced dimerization. The use of FKB-FRB heterodimerization upon addition of rapamycin simulates perfect colocalization on a target cell surface. Here the split site 90-91 for SpyCatcher003 was tested, which showed background reactivity of SpyN⁹⁰⁻¹¹³-NpuN^{cage}-FRB as it retains most of the active fold. SpyN¹⁻⁹⁰-NpuN^{cage}-FKBP and FRB-NpuC^{cage}-SpyC⁹¹⁻¹¹³ were fused to a FLAG and Myc tag respectively, His₆-SpyTag003 was used as a probe. SpyCatcher003 alone or incubated with the probe was used as a positive control for splicing or SpyCatcher003-SpyTag003 reactivity, respectively.

FIG. 10A. An NrdJ-1 fusion was prepared and characterized; image of SDS-PAGE is shown.

FIG. 10B. System of FIG. 10A with RP-HPLC analysis of NrdJ-1 characterizing the purity of the preparation shown.

FIG. 10C. System of FIG. 10A with the protein further characterized by ESI-TOF MS for mass confirmation.

FIG. 10D. System of FIG. 10A with the protein further characterized by analytical gel-filtration chromatography validating its monomeric status.

FIG. 10E. System of FIG. 10A with circular dichroism spectra displaying the secondary structure of NrdJ-1 sampled at pH 6.5 and 7.2, which are largely superimposable (top). The spectrum at pH 7.2 was used to quantify the ratios of secondary structure elements (assessed by Dichro Web) and compared to that estimated from the crystal structure (bottom).

FIG. 10F. System of FIG. 10A with Ramachandran plot generated in Coot of the crystal structure of NrdJ-1 showing no outliers.

FIG. 11A. The elution profiles of SpyN-αHER2 employing tuned eNrdJ-1N^{cage} variants. The retention volume profiles of all tested tuned variants were determined by size-exclusion chromatography. All variants were sampled in 100 mM sodium phosphate (pH7.2), 150 mM NaCl, 1 mM EDTA at 1 mg/mL equaling 25-26 μM, which is >250-fold greater than the concentrations used in the standard cell assay. The determined hydrodynamic radii correspond to monomeric status when compared to a known set of size standards (from left to right: BSA-dimer (133 kDa); β-amylase (56 kDa); ovalbumin (43 kDa); myoglobulin (17 kDa); the dotted line indicates the theoretical retention volume of 14.27 mL for a globular 40 kDa protein. The retention volume profiles for the SpyN-αHER2 variants employing electrostatically engineered cages are shown.

FIG. 11B. The elution profiles of SpyN-αHER2 employing tuned eNrdJ-1N^{cage} variants with different cage lengths. The retention volume profiles of all tested tuned variants were determined by size-exclusion chromatography. All variants were sampled in 100 mM sodium phosphate (pH7.2), 150 mM NaCl, 1 mM EDTA at 1 mg/mL equaling 25-26 μM, which is >250-fold greater than the concentrations used in the standard cell assay. The determined hydrodynamic radii correspond to monomeric status when compared to a known set of size standards (from left to right: BSA-dimer (133 kDa); β-amylase (56 kDa); ovalbumin (43 kDa); myoglobulin (17 kDa); the dotted line indicates the theoretical retention volume of 14.27 mL for a globular 40 kDa protein. The retention volume profiles for the SpyN-αHER2 variants employing toehold engineered cages are shown. The standard cNrdJ-1N^{cage} has a cage length of 35 amino acids.

FIG. 12A. Systematic screen to identify SpyCatcher003 split site and optimization of SMART-SpyCatcher. The overall goal was to identify a split-site where the N- and C-terminal fragments of SpyCatcher003 would remain inactive towards SpyTag003 on their own or when incubated in their respective pairs, while gaining complete functionality once reconstituted through protein splicing. For this purpose, SpyCatcher003 was split at various sites and the complementary pairs used to generate FLAG-SpyN^{1-x}-NpuN^{cage}-FKBP and FRB-NpuC^{cage}-SpyC^{y-113}, with x and y denoting the last and first residue of the two fragments (i.e., the split site). The lysine involved in the isopeptide bond is highlighted in bold in the upper row of the amino acid sequence shown.

FIG. 12B. Conditional protein splicing was induced either by proteolytical decaging or through chemically induced dimerization. The use of FKB-FRB heterodimerization upon addition of rapamycin simulates ideal colocalization on a target cell surface.

FIG. 12C. The result of screening the fragment pair generated from splitting SpyCatcher003 at position 73-74. Neither SpyN¹⁻⁷³-NpuN^{cage}-FKBP nor FRB-NpuC^{cage}-SpyC⁷⁴⁻¹¹³ had any background reactivity with SpyTag003, while CPS generated a fully competent SpyCatcher003 able to form a covalent complex with the SpyTag003. SpyCatcher003 alone or incubated with the SpyTag003 probe was used as a size standard for splicing and SpyCatcher003-SpyTag003 reactivity, respectively. The legend applies for all subsequent western blots.

FIG. 12D. Npu^{cage} was swapped with NrdJ-1^{cage} giving SpyN¹⁻⁷³-NrdJ-1N^{cage}-FKBP and FRB-NrdJ-1C^{cage}-SpyC⁷⁴⁻¹¹³, which yielded a more robust reaction pair for CPS.

FIG. 12E. Inactivation of NrdJ-1^{cage} by C1A mutation within NrdJ-1N^{cage} prevented both splicing of SpyCatcher003 and reactivity with SpyTag003 displaying the dependence of fragment ligating for gain of function.

FIG. 12F. The crystal structure of fusion NrdJ-1 displays a classical Hedgehog/Intein horseshoe fold. Its 1D representation is shown to indicate the relative N- and C-termini of the split intein fragments and the position of the non-catalytic Cys76.

FIG. 12G. Structural details of Cys76 also showing Leu2 and a relevant cavity in grey (top). The structural effects of introducing C76V was assessed using AlphaFold2 (bottom). The introduced branched side chain of valine is predicted to result in cavity reduction close to position 76 (arrow) and may be accommodated by the rotation of the side chain of Leu2. No other apparent structural consequences anticipated from the structural prediction are evident; there was a low root-mean-square deviation between the two backbones of NrdJ-1 (crystal structure) and NrdJ-1(C76V) (AlphaFold2 prediction) of 0.555 Å.

FIG. 12H. Experimental validation that the C76V mutation has no noticeable impact on the protein switch reactivity with SpyTag003 upon addition of rapamycin.

FIG. 12I. ESI-TOF MS spectra of the spliced SpyCatcher003 validating its mass. All reactions were tested at 1 μM FLAG-SpyN¹⁻⁷³-IntN^{cage}-FKBP, 1 μM FRB-IntC^{cage}-SpyC⁷⁴⁻¹¹³-Myc, and 2 μM SpyTag003-AF594 overnight at 37° C. supplemented with 10 μM rapamycin when specified. While His₆-SpyTag003 was used as a probe initially in FIG. 12C, an Alexa Fluor 594 labeled version was used in all subsequent experiments.

FIG. 13A. Confocal microscopy of K562 cell lines treated with SMART-SpyCatcher for [HER2 AND EGFR] operation. K562^{HER2+/EGFR+} cell line were assayed for SMART-SpyCatcher activation and analyzed by confocal microscopy. In comparison to the untreated negative sample (top row), cells treated with SMART-SpyCatcher (SpyN-αHER2 and SpyC-αEGFR) using NrdJ-1^{cage} has a faint AF594 signal associated with their surface (middle row). The SpyTag003-AF594 recruitment was boosted by employing eNrdJ-1^{cage}, a mutant of NrdJ-1N^{cage} with K104EK119A in the cage of NrdJ-1N^{cage} (giving eNrdJ-1 N^{cage}) and D66K in the cage of NrdJ-1C^{cage} (giving eNrdJ-1 C^{cage}) for the improved actuation of SMART-SpyCatcher (bottom row). Scale bar equals 20 μm.

FIG. 13B. Confocal microscopy of K562 cell lines treated with SMART-SpyCatcher for [HER2 AND EGFR] operation. Single K562 cells were analyzed for the colocalization of HER2-eGFP. EGFR-iRFP and SpyTag003-AF594 signals (scale bars equal 10 μm).

FIG. 13C. The corresponding line plot graphs for the images in FIG. 13D. The double positive cell line K562^{HER2+/EGFR+} has a defined membrane colocalization between the three signals, whereas the single positive cells lack the AF594 signal. Experiments were performed with 100 nM SMART-SpyCatcher (using eNrdJ-1cage) and 100 nM SpyTag003-AF594.

FIG. 13D. K562, K562^{HER2+}, K562^{EGFR+}, and K562^{HER2+EGFR+} cell lines were mixed and assayed as in FIG. 13B. Zoom-ins represent the four cell lines and their respective recruitment of SpyTag003-AF594 upon the described treatment.

FIG. 14A. The mechanism of action of SMART-SpyCatcher. Confocal microscopy of K562^{HER2+/EGFR+} treated with SMART-SpyCatcher as SpyN-αHER2 and SpyC-αEGFR for [HER2 AND EGFR] logic operation (row 1). In subsequent rows the cells were supplemented with DARPins targeting HER2 and EGFR thereby blocking SMART-SpyCatcher binding (row 2), an inactivated version of SMART-SpyCatcher (SpyN-αHER2 with Cys1-alkylated NrdJ-1 N^{cage}) blocking splicing (row 3), or excess unlabeled SpyTag003 blocking the fluorescently labeled probe (row 4).

FIG. 14B. Western-blot analysis of K562^{HER2+/EGFR+} cells treated as indicated. The reconstituted SpyCather003 is seen as an overlay between the FLAG-SpyN-αHER2 and SpyC-αEGFR-Myc signals; the covalent SpyTag003-SpyCatcher003 complex is identified by Strep800 (SpyTag003-biotin probe was used here).

FIG. 14C. Using a labeled, but inactive mutant SpyTag003^{D117A}-AF594 instead of SpyTag003-AF594 led to a loss of signal, signifying the importance of the covalent bond to resist loss of interaction between SpyTag003 and SpyCatcher003.

FIG. 14D. SMART-SpyCatcher was tested on K562^{HER2+/EGFR+} in buffer (DPBS, 2 mM CaCl₂), 1% w/v BSA), or in medium (RPMI 1640, cystine-free) alone or supplemented with 5% v/v FBS.

FIG. 14E. Mixed-population flow-cytometry assay experiment with the four K562 cells lines naïve, single, or double positive for HER2-eGFP and EGFR-iRFP. The mixed population was treated with SMART-SpyCatcher for the AND gate combinations shown on the left aimed at the subpopulations with the targeted profiles. Flow cytometry was then performed to quantify the level of SpyTag003-AF594 recruitment to the various subpopulations. Flow cytometry data are presented as the mean with error bars signifying the standard error mean (n=3 independent biological replicates).

FIG. 14F. Mixed K562, K562^{HER2+}, K562^{EGFR+}, K562^{HER2+/EGFR+} cell lines were treated with SMART-SpyCatcher for [HER2 AND EGFR] logic using eNrdJ-1cage as the actuator (row 1). When an uncaged NrdJ-1 variant was used for actuation massive cell-cell crosslinking between single and double positive cells was observed (rows 2 and 3). Such cross-linking occurs presumably from in-solution, solution-surface, and surface-surface splicing. The resulting cell-cell junctions are visualized by the recruitment of SpyTag003-AF594. Crosslinking was not observed with SMART-SpyCatcher using eNrdJ-1^{cage}, and caging the split intein fragments is therefore critical for maintaining the dependence of antigen binding and co-localization for AND gated activation and to prevent cross-linking artifacts. Scale bars equal 20 μm. Experiments were performed with 100 nM SMART-SpyCatcher (caged or uncaged), and 100 nM SpyTag003-AF594.

FIG. 15A. Structural analysis of NrdJ-1 and electrostatic tuning of eNrdJ-1N^{cage}. The initial association of cognate split intein fragment pairs can be driven by electrostatic steering. Shown is the fit between the electronegative groove of NrdJ-1N and the electropositive N-terminal tail of NrdJ-1C.

FIG. 15B. Structural details of the electrostatic interactions involved in the split intein fragment-fragment association.

FIG. 15C. Structural details of the electrostatic interactions involved in the split intein fragment-fragment association; the lower ribbon arrow corresponds to the residues and chain of NrdJ-1C, and the other ribbon arrows correspond to the residues and chain of NrdJ-1N.

FIG. 15D. Structural details of the electrostatic interactions involved in the split intein fragment-fragment association.

FIG. 15E. Schematic summary of the electrostatic interactions and their characters.

FIG. 15F. Overview of the tested electrostatically tuned eNrdJ-1N^{cage} variants. The activity of SpyN-αHER2 (using the assigned tuned eNrdJ-1N^{cage} variant) with SpyC-αEGFR (using the standard eNrdJ-1C^{cage} variant) was determined by mixed-population flow cytometry.

FIG. 15G. The recruitment of SpyTag003-AF594 to K562^{HER2+/EGFR+} cells was measured upon incubation with each SpyN-αHER2/SpyC-αEGFR pair and normalized to that of a sample using the standard eNrdJ-1^{cage}. All experiments were performed with 100 nM SpyN-αHER2/SpyC-αEGFR, and excess SpyTag003-AF594. Data are presented as the mean with error bars signifying the standard error mean (n=3 independent biological replicates). One-way ANOVA followed by Dunnett's post-hoc test was used for statistical analysis.

FIG. 15H. The relative SpyTag003-AF594 recruitment to the various K562 cell lines as a measure of on-target/off-target fidelity from the experiments presented in FIG. 15G.

FIG. 16A. Structural analysis of NrdJ-1 and cage toehold tuning of eNrdJ-1 N^{cage}. The two chains of the NrdJ-1 fragments (NrdJ-1N and NrdJ-1C) intertwine to form a long horseshoe-like fold that is stabilized by beta-strand hydrogen bonds.

FIG. 16B. Structural analysis of NrdJ-1 and cage toehold tuning of eNrdJ-1N^{cage}. The two chains of the NrdJ-1 fragments (NrdJ-1N and NrdJ-1C) intertwine to form a long horseshoe-like fold that is also stabilized by hydrophobic packing.

FIG. 16C. A schematic overview of the C-terminal end of NrdJ-1 and interactions in which it is involved.

FIG. 16D. Overview of the tested cage toehold tuned eNrdJ-1N^{cage} variants. The activity of SpyN-αHER2 (using the assigned tuned eNrdJ-1N^{cage} variant) with SpyC-αEGFR (using the standard eNrdJ-1C^{cage} variant) was determined by mixed-population flow cytometry.

FIG. 16E. The recruitment of SpyTag003-AF594 to K562^{HER2+/EGFR+} cells was measured upon incubation with each SpyN-αHER2/SpyC-αEGFR pair and normalized to that of a sample using the standard eNrdJ-1^{cage} (cage length 1-35). All experiments were performed with 100 nM SpyN-αHER2/SpyC-αEGFR, and excess SpyTag003-AF594. Data are presented as the mean with error bars signifying the standard error mean (n=3 independent biological replicates). One-way ANOVA followed by Dunnett's post-hoc test was used for statistical analysis.

FIG. 16F. The relative SpyTag003-AF594 recruitment to the various K562 cell lines as a measure of on-target/off-target fidelity from the experiments presented in FIG. 16E.

FIG. 17A. Towards 3-input logic gates with SMART-SpyCatcher. Mixed K562 cells were incubated with SpyN-αHER2, SpyN-αEGFR, SpyC-αHER2, and SpyC-αEGFR to make a [HER2 OR EGFR] logic function, which broadly targets any cell line displaying any of the two antigens.

FIG. 17B. Mixed-population flow cytometry quantifying the outcome from the system described for FIG. 17A, where only wildtype K562 does not recruit SpyTag003-AF594.

FIG. 17C. Flow cytometry data of K562^{HER2+/EGFR+} treated with SMART-SpyCatcher assigned for [HER2 AND EGFR] logic using SpyN-αHER2 with K31 or K31E in the N-terminal fragment of SpyCatcher003.

FIG. 17D. Quantification of the level of SpyTag003-AF594 recruitment as in FIG. 17C. All experiments were performed with 100 nM SMART-SpyCatcher, and 100 nM SpyTag003-AF594. Data are presented as the mean with error bars signifying the standard error mean (n=3 independent biological replicates). A paired t-test was used for statistical analysis (P-value=0.0001).

FIG. 18A. SMART-SpyCatcher uses endogenous surface antigen levels for target discrimination. Phenotyping the relative surface levels of HER2, EGFR, and EpCAM was done for each cell line using targeting DARPins labeled with Alexa Fluor 594. The recruitment of SpyTag003-AF594 was done in response to various SpyN/SpyC combinations for each cell line. Samples were analyzed by flow cytometry to record the AF594 signal intensities.

FIG. 18B. K562^{HER2+/EGFR+} was treated with SMART-SpyCatcher for [HER2 AND EGFR] logic and the recruitment of SpyTag003-AF594 imaged. The cells were stained with LysoTracker (Lyso), which colors acidic compartments. The eGFR and iRFP signals were observed both on the cell membrane and in internal partitions corresponding to late-endosomal or lysosomal compartments. Scale bars equal 10 μm.

FIG. 18C. Multiple cells (n=291) as those in FIG. 18B were used to image quantify the correlation between the eGFP, iRFP and AF594 signals. The use of LysoHunter allowed for the subtraction of background signals from eGFP and iRFP arising from the lysosomal compartments. The increase in the AF594 signal (SpyTag003-SpyCatcher003 complex) is dependent upon the abundance of both HER2 and EGFR.

FIG. 18D. Focused analysis of the K562^{HER2+/EGFR+} subgroup that displayed the 15% highest iRFP signal (n=39) gives a strong association of AF594 with the eGFP signal and a weak association with the iRFP signal. This subpopulation, which displays the highest levels of EGFR therefore has a stronger dependence on the surface levels of HER2 for SpyTag003-AF594 recruitment and hence SMART-SpyCatcher activation. In contrast, the K562^{HER2+/EGFR+} subgroup, which displayed the 10% highest eGFP signal (n=30) shows a weak association between AF594 and eGFP but a moderate association with iRFP. This subpopulation, which displays comparatively higher levels of HER2, but lower levels of EGFR, is therefore limited by the availability of EGFR to enable SMART-SpyCatcher activation and SpyTag003-AF594 recruitment.

FIG. 18E. The total cellular level of HER2 and EpCAM for MCF-10a, MCF-7, and Sk-br-3 was detected by western blotting.

FIG. 18F. Image quantification based on the western blot results from FIG. 18E. The values were corrected against the GAPDH signal for each cell line and normalized to the overall highest level (Sk-br-3 for HER2, MCF-7 for EpCAM).

FIG. 18G. MCF-10a and MCF-10a stained with CMFDA were phenotyped for any differences in the levels of detectable surface HER2 and EpCAM to confirm that the staining had no effect. Data are presented as the mean with error bars signifying the standard error mean (n=3 independent biological replicates).

FIG. 19A. Highly-selective hapten delivery and recruitment of anti-hapten antibodies. Following AND gated reconstitution of SpyCatcher003, cells can be surface decorated with SpyTag003 conjugated to dinitrophenol (SpyTag003-DNP) to recruit anti-DNP antibodies.

FIG. 19B. Confocal microscopy of K562^{HER2+/EGFR+} treated with SMART-SpyCatcher assigned for [HER2 AND EGFR] logic, then SpyTag003-DNP and rabbit anti-DNP IgG (row 1). The anti-DNP antibody was detected using a goat anti-rabbit IgG AF594 conjugate. In subsequent samples, cells were supplemented with αHER2 and αEGFR DARPins blocking SMART-SpyCatcher binding (row 2), an inactivated version SMART-SpyCatcher (SpyN-αHER2 with Cys1-alkylated eNrdJ-1N^{cage}) blocking splicing (row 3), or excess unlabeled SpyTag003 blocking the dinitrophenyl probe (row 4).

FIG. 19C. A mixture of K562 cell lines (K562^{EpCAMlo}, K562^{EGFR+/EpCAMlo}, K562^{HER2+/EpCAMlo} and K562^{HER2+/EGFR+/EpCAMlo}) was used to test for the selective recruitment of SpyTag003-DNP upon SMART-SpyCatcher treatment. The mixed population was incubated with the indicated SMART-SpyCatcher combination and SpyTag003-DNP, then rabbit anti-DNP IgG. Recruitment of the anti-DNP antibody was detected as in FIG. 19B. Quantification of the AF594 signal was done by flow cytometry. The antigen profile of each cell line is indicated below each bar plot. All experiments were performed with 100 nM SMART-SpyCatcher, and 100 nM SpyTag003-DNP. Data are presented as the mean with error bars signifying the standard error mean (n=3 independent biological replicates).

FIG. 19D. Combinations of mixed K562 cell lines were used to test for the recruitment of SpyTag003-DNP upon SMART-SpyCatcher treatment (mixed population 1 was K562^{EpCAMlo}, K562^{EGFR+/EpCAMlo}, K562^{HER2+/EpCAMlo} and K562^{HER2+/EGFR+/EpCAMlo}; mixed population 2 was K562^{EpCAMlo}, K562^{EGFR+/EpCAMlo}, K562^{HER2+/CAMhi}, and K562^{HER2+/EGFR+/EpCAMhi}). The mixed populations were incubated with the indicated SMART-SpyCatcher combination and SpyTag003-DNP, then rabbit anti-DNP IgG. Recruitment of the anti-DNP antibody was detected as in FIG. 19B. Quantification of the AF594 signal was done by flow cytometry. The antigen profile of each cell line is indicated below each bar plot. All experiments were performed with 100 nM SMART-SpyCatcher, and 100 nM SpyTag003-DNP. Data are presented as the mean with error bars signifying the standard error mean (n=6 independent biological replicates for K562^{EpCAMlo} and K562^{EGFR/EpCAMlo} and n=3 independent biological replicates for all other cell lines).

FIG. 20A. Targeted cell depletion using Boolean logic. Timeline of the two strategies used. A dose consisted of cells treated with 100 nM SMART-SpyCatcher and 100 nM SpyTag003-biotin followed by a wash. The cells were then cultured in complete media supplemented with 20 nM Streptavidin-Saporin conjugate.

FIG. 20B. Analysis of the mixed K562 cell lines was done by flow-cytometry to determine the viability of the individual cell lines normalized. A one-dose regiment is used here.

FIG. 20C. The three AND gates used in the depletion study of A431 cells.

FIG. 20D. For the analysis of A431 viability a formarzan-based assay was used to measure the metabolic turnover of XTT. The developed absorbance signal at 465 nm was read on a plate reader. A two-dose regiment is used here.

FIG. 21A. Protein delivery and proximity protein labeling. Western blot analysis of K562^{HER2+EGFR+} treated as indicated using SpyN-αHER2 and SpyC-αEGFR as SMART-SpyCatcher. SpyTag003-APEX2 carries an HA tag.

FIG. 21B. K562^{HER2+/EGFR+} was treated with SMART-SpyCatcher operating through [HER2 AND EGFR] logic, then incubated with SpyTag003-APEX2, washed and finally labeled with biotin phenol using H₂O₂ to initiate the reaction, which was followed over time. The optimal time point of labeling was estimated as 4 min, with maximal signal-to-noise ratio.

FIG. 21C. A similar strategy as that for FIG. 21B was applied to the OE19 cell line using SpyN-αHER2/SpyC-αEpCAM. The optimal timepoint for APEX2 labeling of OE19 was 2 min.

FIG. 21D. A similar strategy as that for FIG. 21B was applied to the A431 cell line using SpyN-αEGFR/SpyC-αEpCAM. The optimal timepoint for APEX2 labeling of A431 was 4 min.

FIG. 22A. Illustration of percentage of drugs targeting the surfaceome; illustration of percentage of the proteome that is the surfaceome.

FIG. 22B. Illustration of the mono-targeting approach (targeting a single surface antigen).

FIG. 22C. Illustration of an on-target/on-tumor result.

FIG. 22D. Illustration of an on-target/off-tumor result.

FIG. 22E. Illustration of a Boolean AND gate with two possible antigens as inputs.

FIG. 22F. Illustration of result of AND-gated targeting on single-antigen (single positive) cells.

FIG. 22G. Illustration of result of AND-gated targeting on a two-antigen (double positive) cell.

FIG. 23A. Illustration of AND-gated delivery of a fluorophore (star symbol) to a cell expressing on its surface both HER2 and EGFR (with no delivery to cells that express neither or only one of HER2 and EGFR).

FIG. 23B. Flow cytometry plot representing the gating strategy for the separation of the mixed K562 population (K562, K562^{HER2+}, K562^{EGFR+}, K562^{HER2+/EGFR+}) into the individual cell lines for analysis. The flow cytometry gating relied on the fluorescence signal of eGFP and/or iRFP. Equal numbers of cells from each cell line were used in the standard experiment.

FIG. 23C. Graphs showing binding to cells that express both HER2 and EGFR (and not to cells that express neither or only one of HER2 and EGFR).

FIG. 24A. Illustration showing that by using the split-actuator system to implement an AND gated delivery strategy, a toxic payload is delivered to and kills a target cell that displays both target antigens on its surface.

FIG. 24B. Illustration showing experimental setup for evaluating the AND gated delivery strategy.

FIG. 24C. Graphs showing that by using the split-actuator system to implement an AND gated delivery strategy, a toxic payload is delivered to and kills target cells that display both target antigens HER2 and EGFR on their surface, whereas cells that display only one antigen (either HER2 or EGFR) or no antigen (neither HER2 nor EGFR) on their surface are spared.

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

Cell differentiation and tissue specialization lead to unique cellular surface landscapes, while exacerbated or loss of expression patterns can be a source of further heterogenicity often linked to pathological phenotypes¹⁻³. Immunotherapies and emerging protein therapeutics seek to exploit such differences by engaging cell populations selectively based on their surface markers. Because a single surface antigen rarely defines a specific cell type^{4,5}, smart molecular systems that integrate multiple cell surface features to convert on-target inputs to user-defined outputs can be useful. Herein is described an autonomous decision-making protein device driven by proximity-gated protein trans-splicing that allows local generation of an active protein from two otherwise inactive fragments. This protein actuator platform can perform various Boolean logic operations on cell surfaces, allowing highly selective recruitment of enzymatic and cytotoxic activities to specific cells within mixed populations. Due to its intrinsic modularity and tunability, this technology may be compatible with different types of inputs, targeting modalities and functional outputs, and as such has application in the synthetic biology and biotechnology areas.

66% of drugs deposited on the DrugBank target the surfaceome, a minor group (14.5%) of proteins, all of which reside on the cell surface, readily exposed to the extracellular environment. The surfaceome aids in cell-to-cell communication, cell-adhesion, and immune functions, in addition to exhibiting diverse enzymatic and transport functionalities. For this reason, surfaceome members are often described as oncogenic, as perturbations in their activity or abundance can lead to severe pathologies such as cancers.

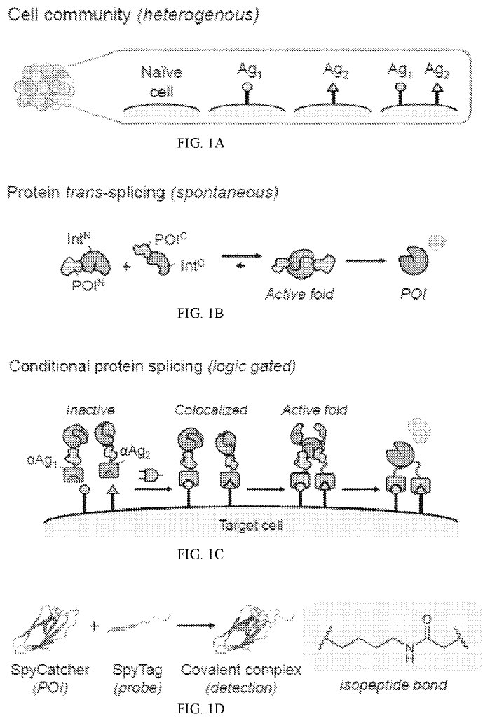

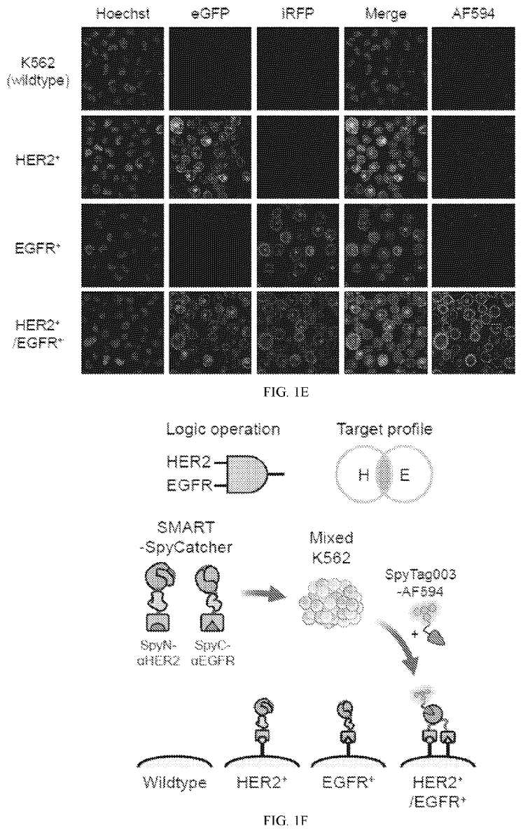

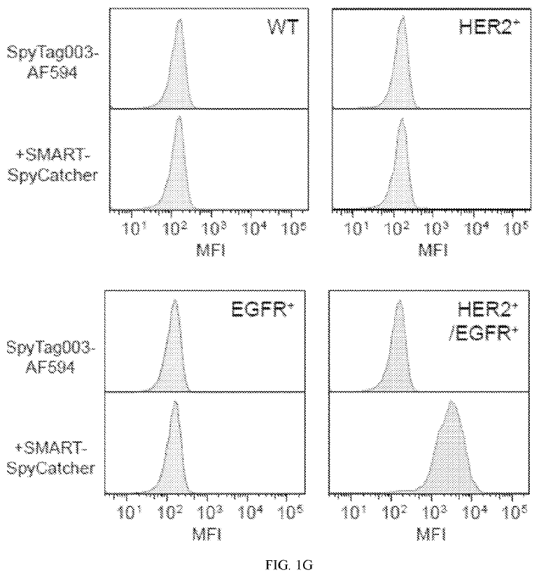

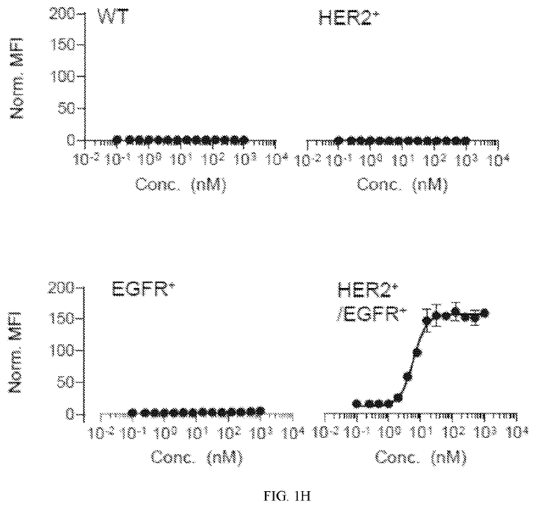

Exploiting the heterogeneity of cell surfaces is used in certain diagnostic and therapeutic approaches. These modalities may employ an agent including a targeting vector such as an antibody that engages a cell surface marker to deliver pharmacological, enzymatic or cellular activityl²⁻¹⁵. Although this approach may succeed in certain situations, single antigens rarely define single cell or tissue types and are often not disease specific (FIG. 1A). That is, a wrong commitment of such an agent can consequently occur, and in the case of therapeutic modalities may lead to unwanted side-effects^{4,5}.

For example, modern anti-cancer immunotherapies may target tumors by binding selectively to surface antigens, such as those constituting the surfaceome. However, very few surface antigens are tumor-specific, and healthy and cancerous tissues can share overlapping antigen profiles. On-target/off-tumor binding can therefore lead to serious side-effects by the elimination of otherwise healthy tissue.

A solution to this problem presented herein is to employ targeting vectors that recognize two or more surface antigens through Boolean logic^{6,7}. Bispecific antibodies partition cell engagement into two concurrent low-affinity binding events; however, achieving complete subpopulation discrimination can be difficult to fine-tune and the type of logic operations possible with these systems is limited. Protein switches and engineered antibody platforms may couple functional outputs to multiple binding events through Boolean logic operations⁸⁻¹¹. However, these platforms are prone to activate in-solution prior to target binding and remain limited in output functions. Smart protein devices that can perform autonomous decision-making to convert on-target inputs to user-defined outputs in a highly spatial and temporal manner may provide absolute cell discrimination in terms of binding and activity.

Protein trans-splicing (PTS) is intrinsically AND logic gated: split intein fragments first undergo complementation followed by an autocatalytic excision-ligation reaction that reconstitutes a protein of interest (POI) in a traceless manner (FIG. 1B)¹⁶. However, many naturally split inteins conceal their AND gated nature by assembly with pico-to-nanomolar dissociation constants, essentially making them indistinguishable from single chain variants with pseudo first-order reaction kinetics¹⁷. Caged split inteins where each fragment is fused to truncated versions of the matching partner have been developed¹⁸. This inhibits fragment complexation of the otherwise fast and efficient naturally split inteins Npu, Gp41-1, GP41-8, and NrdJ-1 and alters their trajectories to follow second-order reaction kinetics. Triggered colocalization can induce spontaneous intermolecular domain swapping, which generates the functional intein fold¹⁹. Therefore, a fragmented POI produced through this scheme is inherently AND gated as it depends on conditional protein splicing (CPS) to recapitulate its function.

Presented herein is the SMART (Splicing-Modulated Actuation upon Recognition of Target) platform that performs autonomous decision making based on Boolean logic without any user manipulation to trigger it. An embodiment of this platform is a device that includes two components, each having three modules; that is, a component includes a caged split intein fragment fused to a split-POI and an antigen-binding domain (e.g., an antibody, an antibody paratope, a T-cell receptor, a complement protein, a nanobody, a DARPin, a ligand, a fragment of these, etc.). The SMART device passively discriminates among various populations based on a population's antigen surface presentations and co-engages cells with a correct combinatorial display prompting splicing and POI function (FIG. 1C). Following this outline, a generalizable scaffold is generated that is able to perform autonomous decision making that implements Boolean AND. OR, NOT, and other logic operations within a heterogenous cell community at single-cell resolution and without any noticeable off-target effects.

For example, cell specific protein actuators using Boolean logic gated protein trans-splicing are set forth herein. In a protein engineering process conditional split inteins are fused to two distinct targeting elements which then direct them to specific cell types expressing two distinct (complementary) surface antigens. This cell-type specificity results in the split-actuator systems according to the invention having potential applicability in cell-type targeted therapies or diagnostics. This induces an on-cell protein trans-splicing reaction, the split inteins functioning as an actuator, which leads to the localized production of a user-selected protein output.

An embodiment of the invention includes the design of a caged version of a split intein, such as a split NrdJ-1 intein, that only undergoes protein trans-splicing splicing upon induced proximity of the two caged split intein fragments. These split inteins can be engineered to ensure tight conditionality and to work efficiently in the oxidizing extra-cellular environment. The caged inteins can be fused to any two targeting vectors (proteins, small molecules, nucleic acids) that engage cell surface antigens, as well as any split (and inert when split) version of a protein payload. Upon binding and receptor clustering, protein splicing ensues leading to the generation of the new functional protein payload on the cell surface. The system can be designed to actuate (work) only when each of two (both) antigens are on the cell surface, so that it operates as a Boolean logic AND gate. In other embodiments, a split intein may be configured to act as another Boolean logic gate, such as an OR gate, a NOT gate, a NOR gate, and/or a gate with more than two inputs.

Embodiments of the invention can tag specific cell types (for example, those expressing two distinct antigens) with a range of payloads including, for example, an antibody epitope (to stimulate a localized immune response), a protein toxin (to kill the target cell), a compound that fluoresces (a fluorophore, to detect the cell type), and/or a peptide or protein, such as a protein ligand for a cell surface receptor. Because embodiments of the invention can work as a logic gate (such as an AND gate), embodiments of the invention can reduce off-target effects associated with cellular therapies, thereby expanding therapeutic windows for biologics.

A process according to the invention incorporates conditional protein splicing. The payload can be entirely recombinant, expressed and purified from archaeal, bacterial, or eukaryotic cell lines, can be semisynthetic, or can be entirely synthetic, with the targeting vector conjugated to the remainder of the system (that is the caged intein actuator and the payload) using a bioconjugation method. Once generated, the components can be added at an appropriate dose to the target cells or tissue in an ex vivo or in vivo application. For example, in vivo application can be to immune privileged organs and tissues, such as the eye and the central nervous system.

The mechanism of action of the designed systems (e.g., split-actuator systems) according to the invention has been validated (confirmed), as discussed in the following examples. The ability of the systems to activate specifically on a target cellular subpopulation within a complex mixture containing other non-target cell lines that lack one, both, or more target surface antigens has been quantified. Extending on this, it has been shown that the system works on several cell lines expressing various combinations of cell surface antigens (e.g., HER2+EGFR, HER2+EpCAM, or EGFR+EpCAM). The system has been employed in the cell-type specific generation of different protein payloads that elicit cytotoxic responses or a display (for imaging) and may elicit an immunological response.

The systems according to the invention have utility in biology, synthetic biology, and medicine, including for applications such as research, diagnostics, cell-type targeted therapies, targeted cancer therapies, and CAR-T (chimeric antigen receptor T-cell) therapies and applications.

### Examples

### Engineered Protein Switch

A SMART device can convert two antigen inputs, when presented on the same cell, into a unique protein dock. This allows monitoring and characterization of the device using fluorescence-based methods and delivery of a broad arsenal of functionalities. For example, in a SMART version of SpyCatcher003, isopeptide bond forming reactivity with the sixteen amino acid peptide SpyTag003 (FIG. 1D) is regulated by AND gated CPS²⁰.

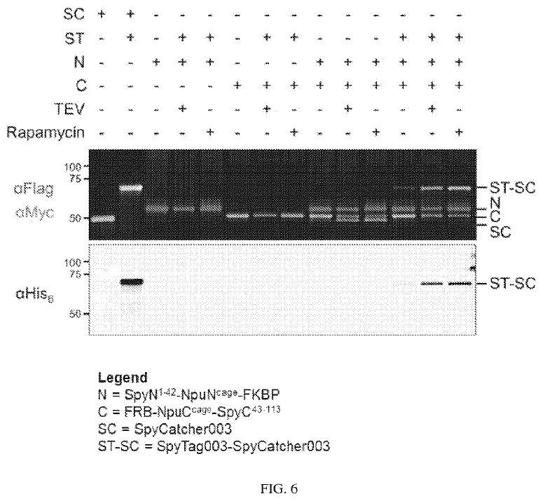

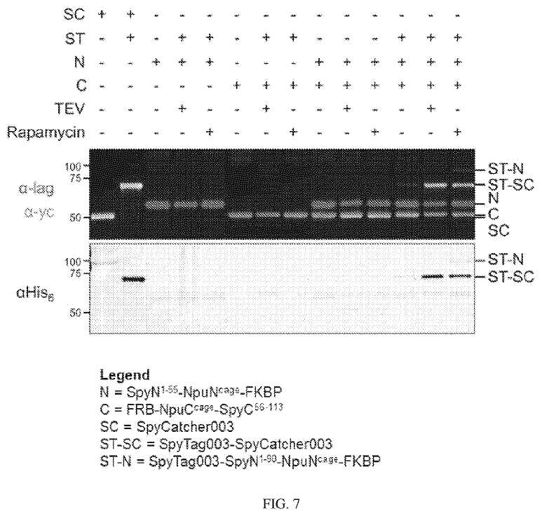

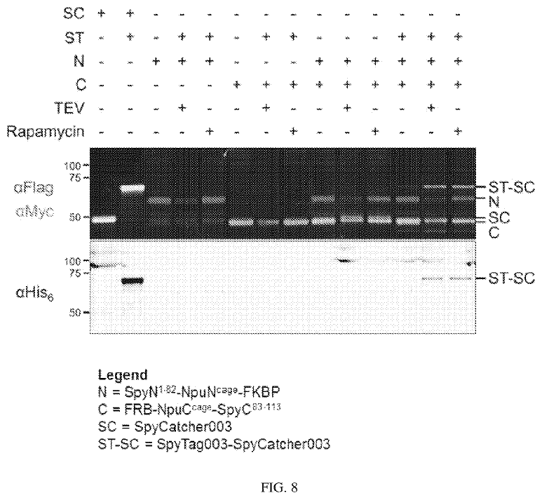

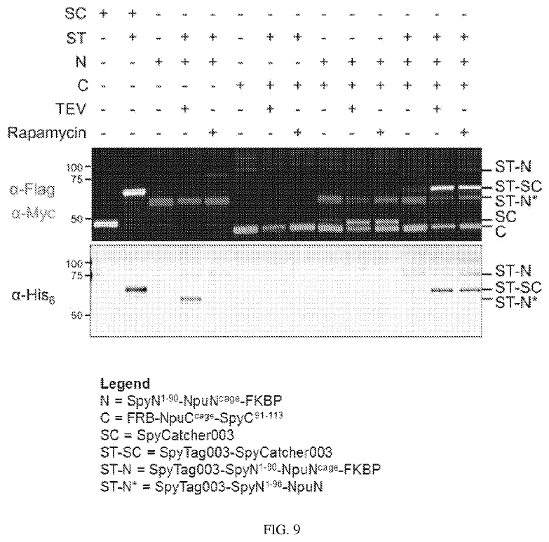

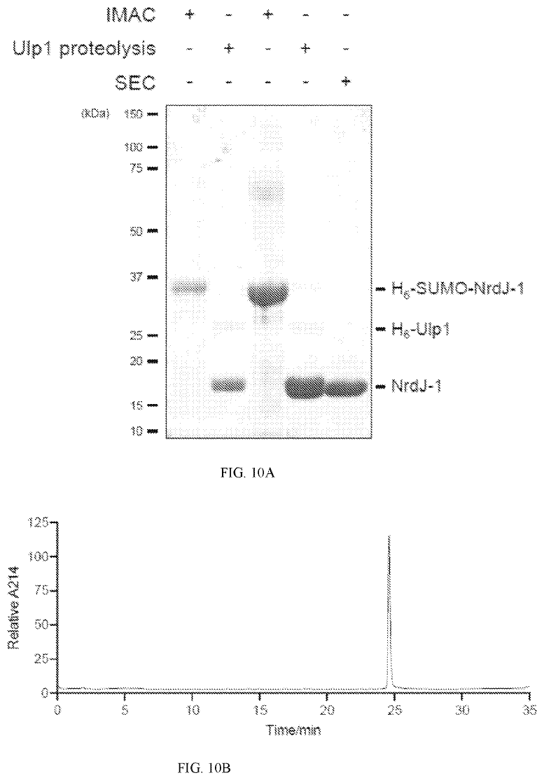

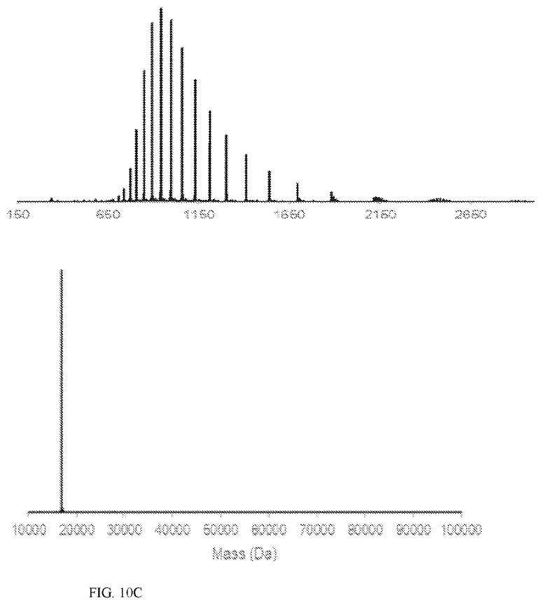

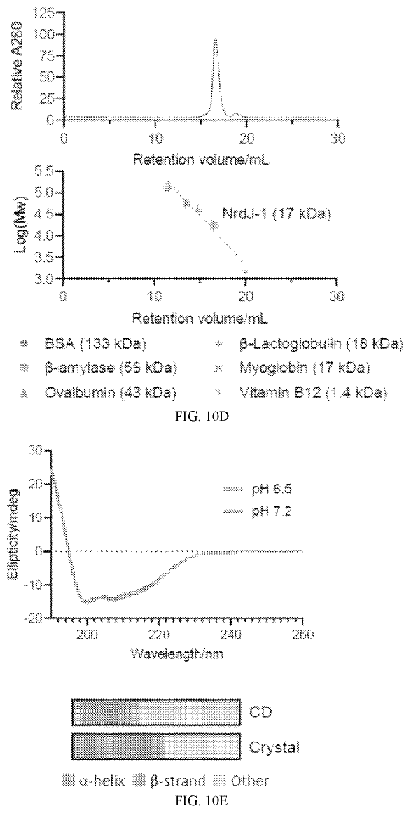

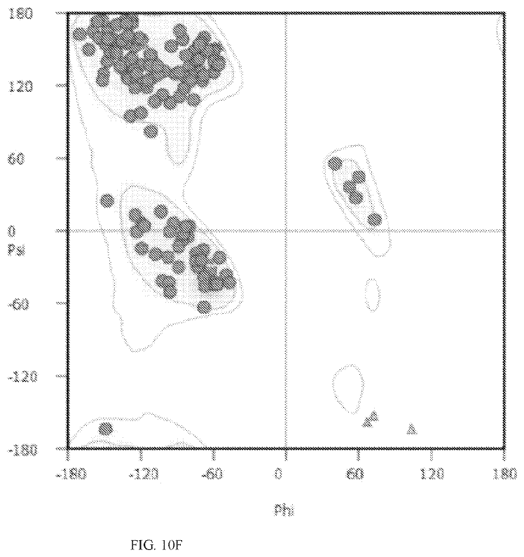

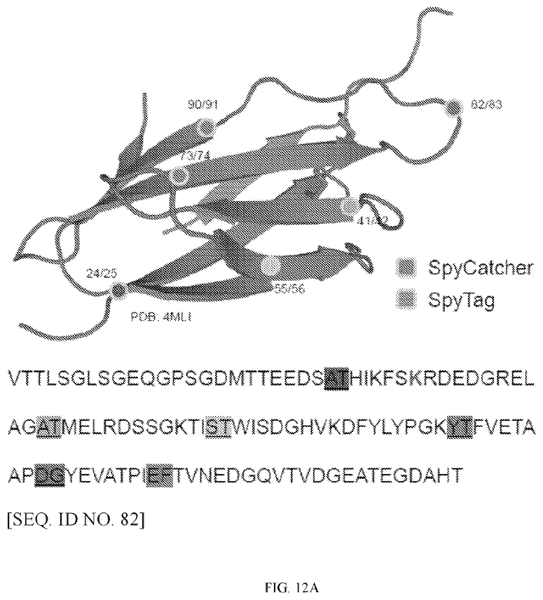

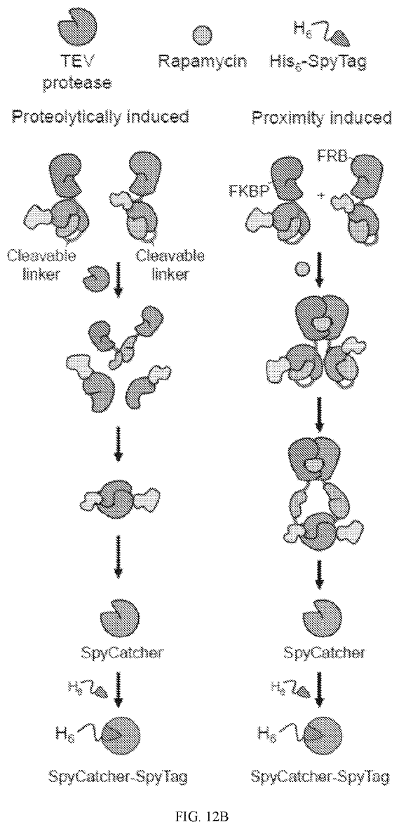

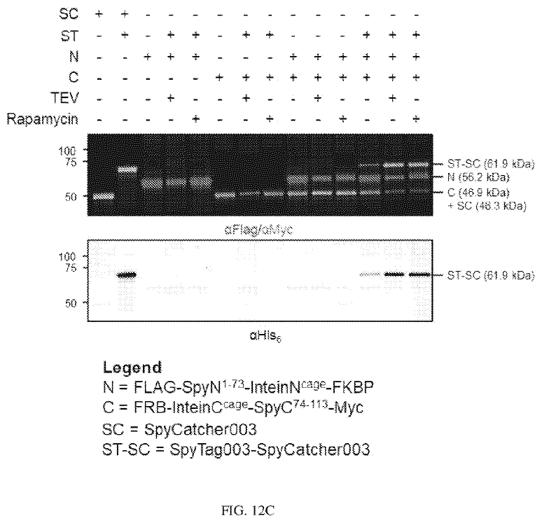

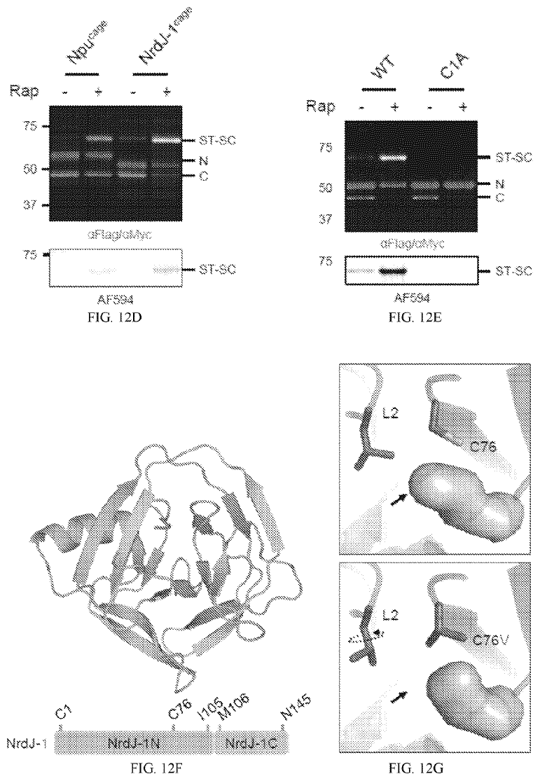

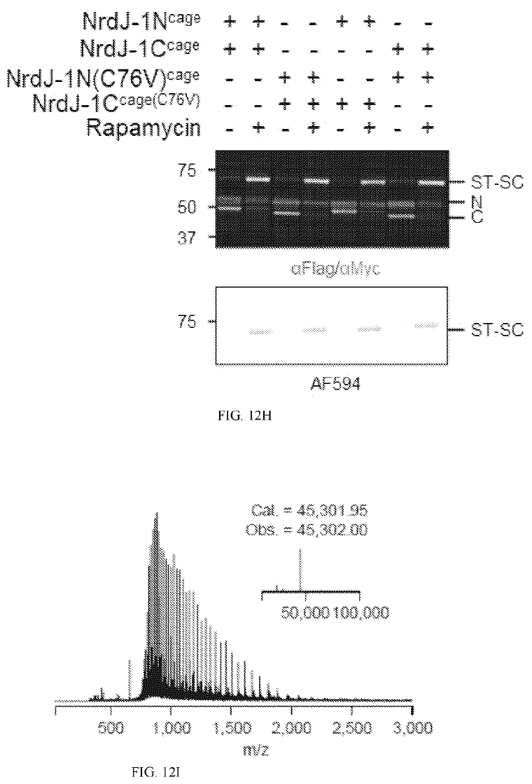

No split version of SpyCatcher003 has previously been reported. Multiple split pairs were systematically screened using Npu^{cage} for CPS (FIG. 12A). Simulating cell-surface colocalization, dimerization of SpyN-NpuN^{cage}-FKBP and FRB-NpuC^{cage}-SpyC was induced by the addition of rapamycin (FIG. 12B). This screen revealed several pairs with an ability to fully recapitulate SpyCatcher003-SpyTag003 reactivity upon splicing (FIGS. 5-9). SpyN¹⁻⁷³ and SpyC⁷⁴⁻¹¹³ had minimal background reactivity with the peptide probe prior to splicing (FIG. 12C). Exchanging Npu^{cage} for NrdJ-1^{cage} gave SpyN-NrdJ-1N^{cage}-FKBP and FRB-NrdJ-1C^{cage}-SpyC, which yielded near-complete splicing upon addition of rapamycin (FIG. 12D), whereas no splicing nor reactivity with SpyTag003 was observed when the cage split intein was inactivated (FIG. 12E). Whereas Npu^{cage} has 4 cysteines in its structure and leaves a cysteine scar, NrdJ-1^{cage} has only 2 cysteines and leaves a serine at the splice site. A fusion of NrdJ-1 was prepared (FIGS. 10A-10F) and its crystal structure solved at 1.95 A resolution (FIG. 12F, Table 3, and following X-ray crystallography section). Using the structural information, we substituted the non-catalytically Cys76 in NrdJ-1^{cage} (FIG. 12G) and found C76V to have no observable effect on CPS (FIGS. 12H-12I), while reducing the number of cysteines in our protein switch to only Cys1. Hereafter, we refer to the individual components, SpyN-NrdJ-1N (C76V)^{cage} and NrdJ-1C^{cage(C76V)}-SpyC as SpyN and SpyC, respectively, and their sum as SMART-SpyCatcher.

### On-Cell Performance and MoA

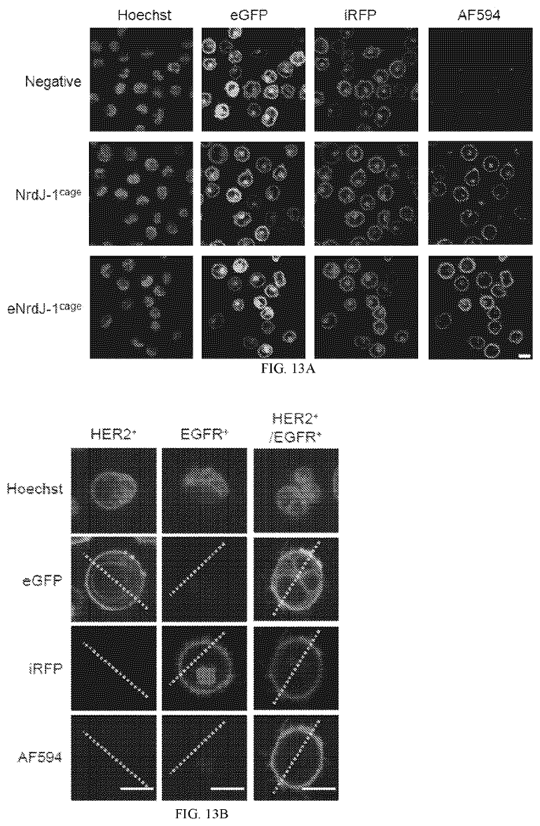

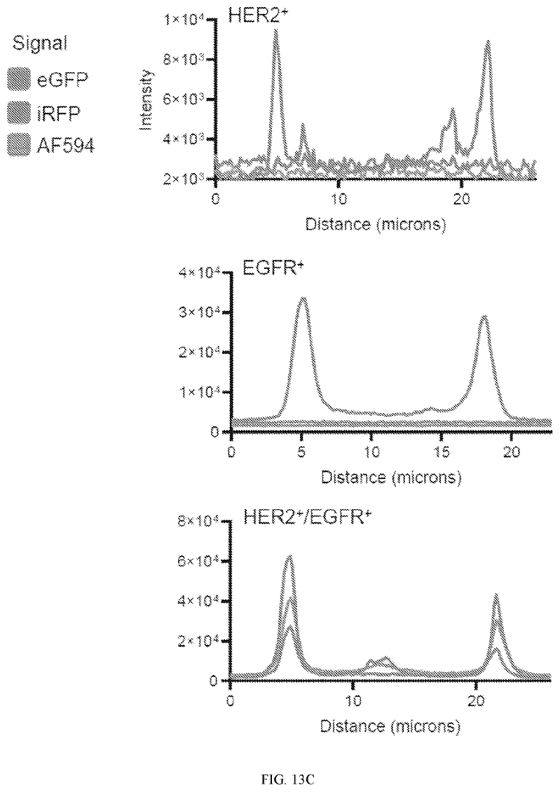

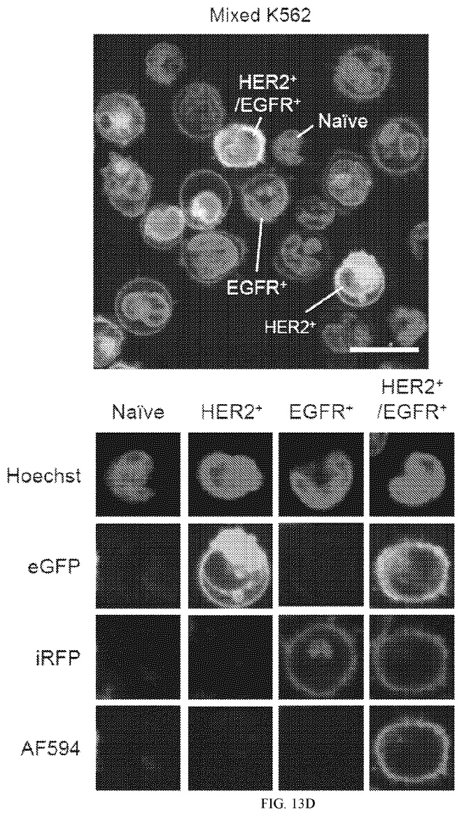

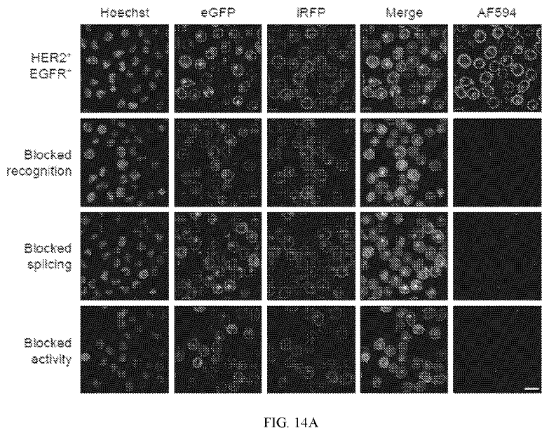

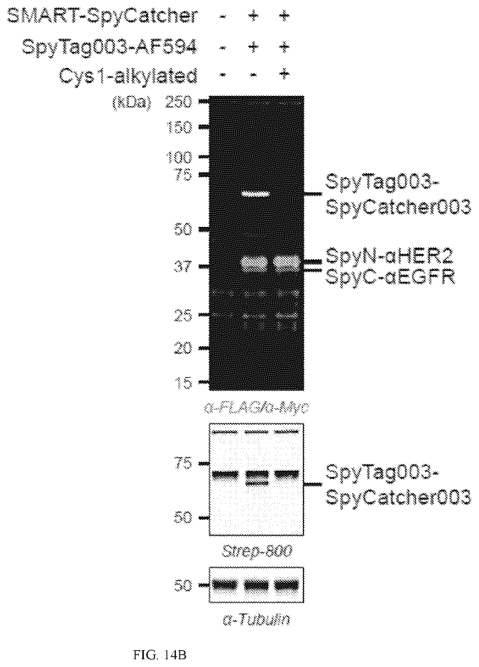

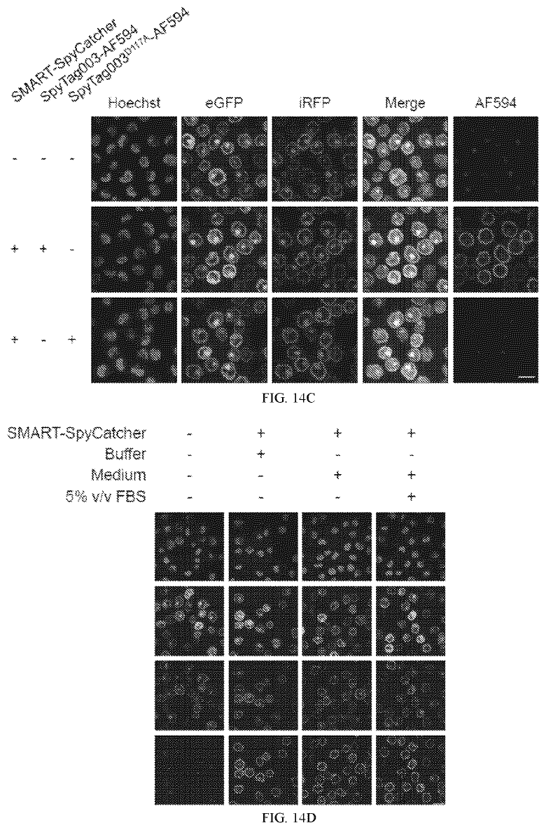

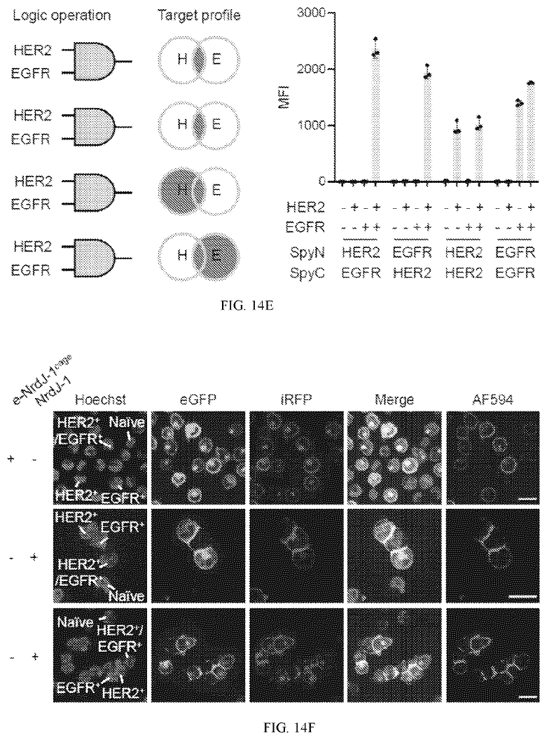

Next. SMART-SpyCatcher was refitted for on-cell activation by removing FKBP and FRB, while installing DARPins²¹⁻²³ targeting the two model antigens HER2 and EGFR, thereby generating SpyN-αHER2 and SpyC-αEGFR. The ability of SMART-SpyCatcher to perform [HER2 AND EGFR] logic was assessed on K562 leukemia cells co-expressing HER2-eGFP and EGFR-iRFP⁸. A dim AF594 signal associated with the cells was observed, and an enhanced version of NrdJ-1^{cage} (eNrdJ-1^{cage}) with a loosened cage to facilitate an increased recruitment of SpyTag003-AF594 was tested. eNrdJ-1^{cage} includes eNrdJ-1N^{cage} and eNrdJ-1C^{cage}, eNrdJ-1N^{cage} results from the substitutions C76V, K104E, and K119A to NrdJ-1N^{cage}; eNrdJ-1C^{cage} results from the substitutions D66K and C76V to NrdJ-1C^{cage}. Although C76 resides in NrdJ-1N, it is part of the cage linked to NrdJ-1C^{cage}. Both caged split intein fragments therefore have this mutation in the enhanced variant pair. This resulted in a noticeably brighter AF594 fluorescence on the cell surface (FIG. 1E, FIG. 13A). To rule out in-solution or at solution-surface interphase activation, the activities of SMART-SpyCatcher on K562 cell lines naïve (wildtype) or single positive for either HER2-eGFP or EGFR-iRFP were evaluated; each of these failed to elicit any response (FIG. 1E, FIGS. 13B-13C). The on-target/off-target specificity of SMART-SpyCatcher when presented simultaneously with multiple decisions, using a mixed-population flow-cytometry assay for quantification, was tested (FIG. 1F). SMART-SpyCatcher retained its target specificity in this setting (FIGS. 1G and 13D) even over a broad concentration range (nanomolar to micromolar), producing a sigmoidal dose-response only for K562^{HER2+/EGFR+} (FIG. 1H). Without being bound by theory, further experimental evidence supported a mechanism of action, in which 1) SpyN-αHER2 and SpyC-αEGFR colocalization, 2) splicing of SpyCatcher003, and 3) reactivity with SpyTag003 are all required, because blocking any of these steps led to a complete loss of the AF594 signal (FIGS. 14A-C). Additionally, while SMART-SpyCatcher works under various conditions (FIG. 14D) and is AND gated (FIG. 14E), its decision-making ability is lost when the cages are omitted from NrdJ-1, instead leading to massive crosslinking between single- and double-positive K562 cells (FIG. 14F).

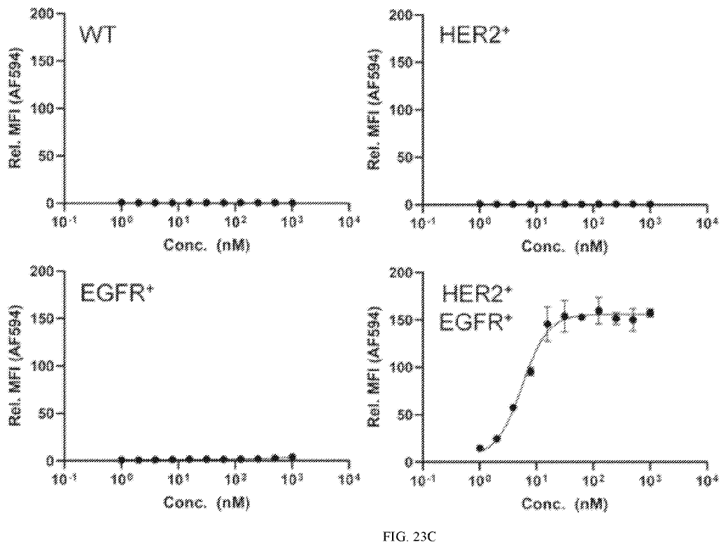

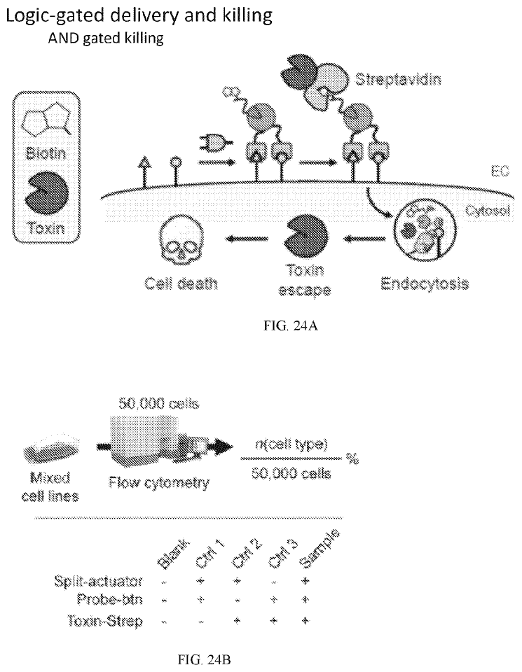

That is, the SMART-SpyCatcher (example of a split-actuator) system was tested on K562 leukemia cell lines that were either naïve or single or double positive for HER2 and EGFR. Fluorescence phenotyping was used to distinguish between the different cell lines using imaging (confocal microscopy) and flow cytometric methods. When the individual cell lines were supplemented with the SMART-SpyCatcher system and a fluorescent probe (SpyTag003-AF594), clear activation was found only on double positive cell lines as intended by the AND gating strategy (FIGS. 23B-23C). Activation was further dependent upon the ability to splice, because inactivating the intein prevented (abolished) probe capture. Dose-response assays with mixed cell lines further indicated the necessity of a cell having both target antigens for proper SMART-SpyCatcher system activation. Only the double positive cell line was associated with robust split-actuator activation over a broad concentration range with minimal to no off-target activation (FIGS. 23B-23C). The split-actuator system was expanded to generate AND gates with inputs selected from HER2, EGFR, and EpCAM and has been used on additional cell lines.

Imaging agents other than or in addition to AF594 can be used to image a target cell line in a heterogeneous population. For example, an imaging agent can be a dye, a protein, a radionuclide, a radiocontrast agent, or a magnetic contrast agent for magnetic resonance imaging (MRI). Imaging can be used, for example, to diagnose a hyperproliferative disorder, such as a benign tumor, malignant tumor, or cancer.

### Tuning Response Dynamics

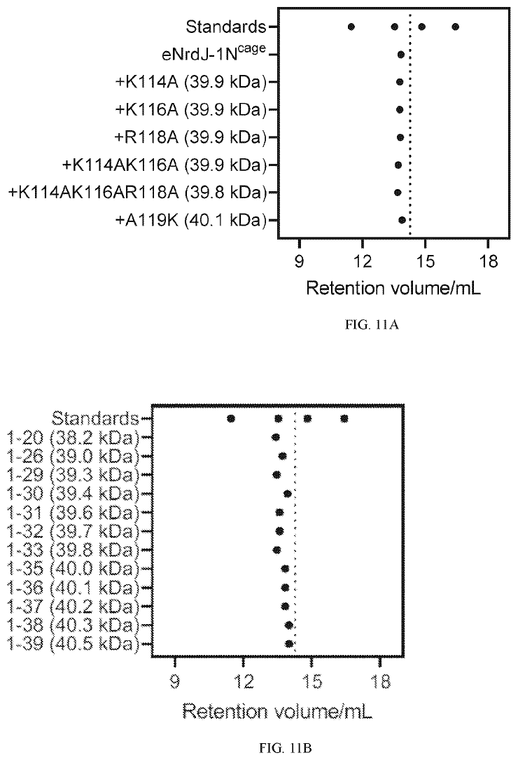

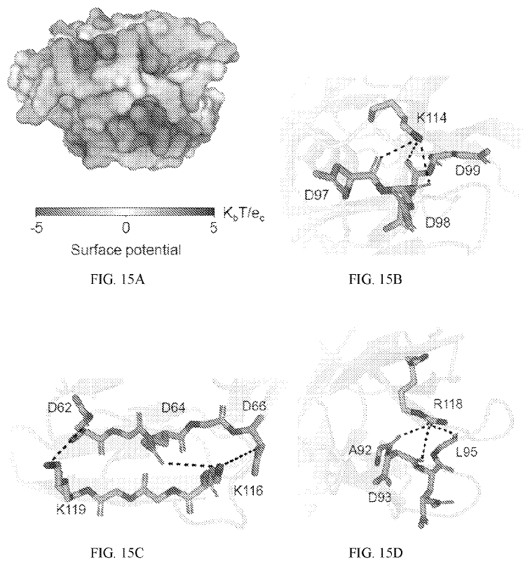

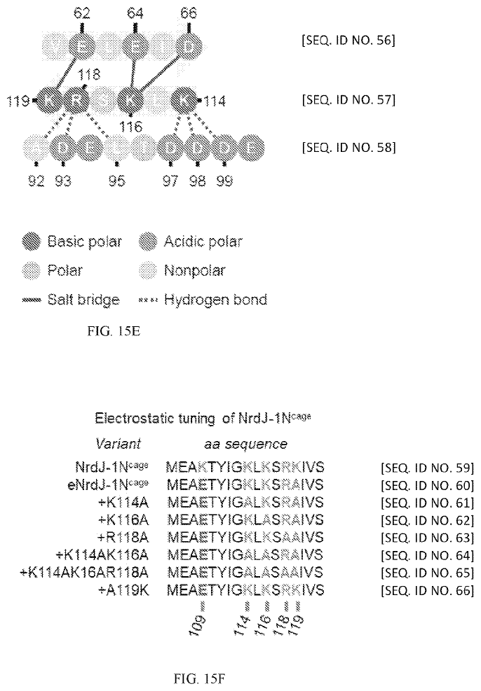

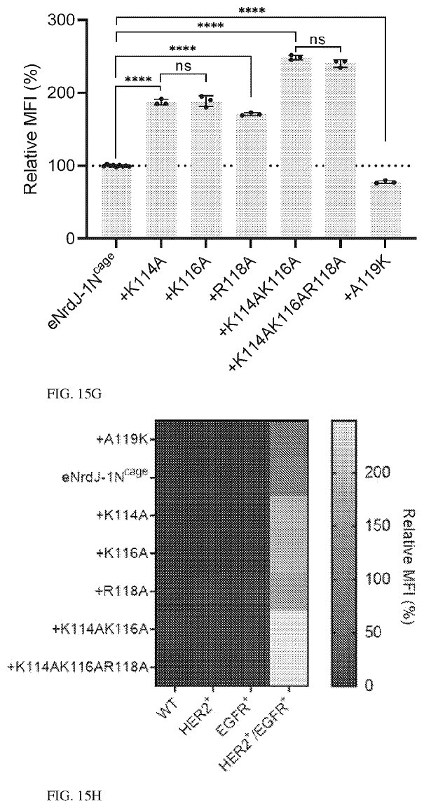

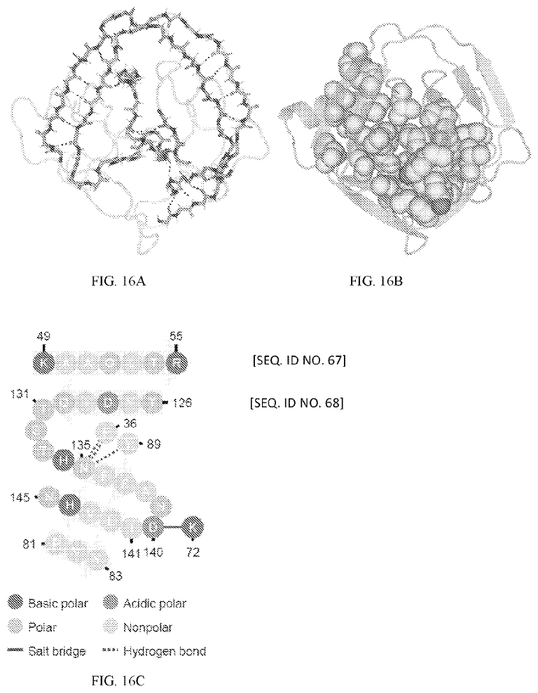

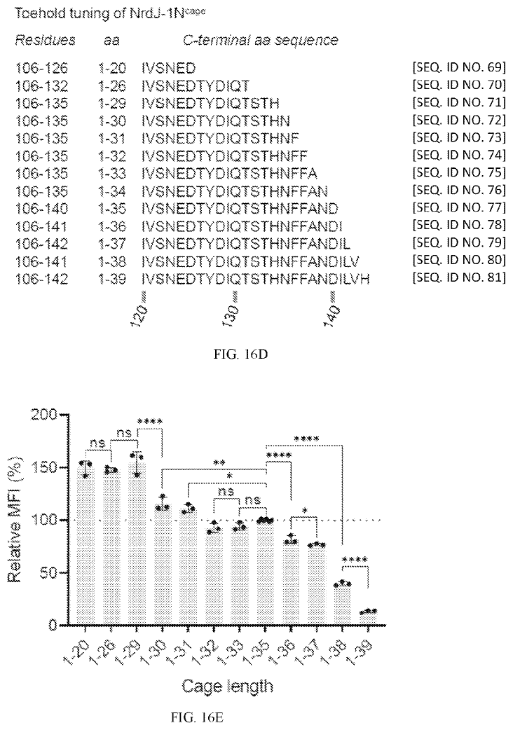

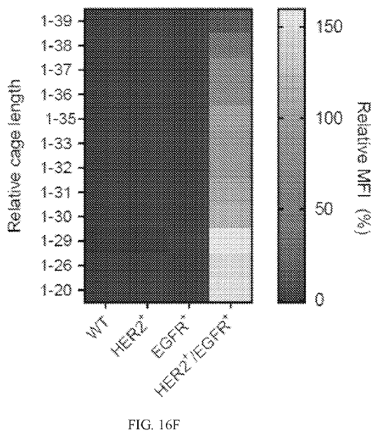

Encouraged by the ability of the protein switch to make correct decisions, a suite of SMART-SpyCatchers with a dynamic response range was created. Guided by the crystallographic information of NrdJ-1, eNrdJ-1N^{cage} I was manipulated further (FIGS. 15A-F), while eNrdJ-1C^{cage} was continued to be used. This afforded multiple pairs with increased or decreased activities (FIG. 15G), without any compromise to target specificity (FIG. 15H). For instance, introducing K114AK116A increased SpyTag003-AF594 recruitment by 250%, while introducing A119K gave a 25% drop. Alternatively, tuning the cage length of eNrdJ-1N^{cage} by either elongating or shortening it (FIGS. 16A-16D) led to variants with activities ranging between 13%-150% of that of the standard 35 amino acid toehold (FIG. 16E) while retaining target fidelity (FIG. 16F). Furthermore, all tuned variants kept their monomeric status with similar Stokes radii (FIGS. 11A-11B).

### Exploring Antigen Limitations

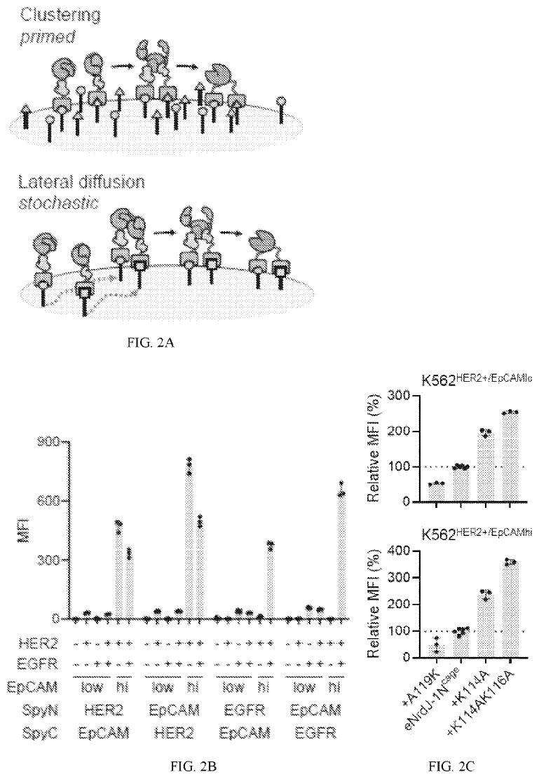

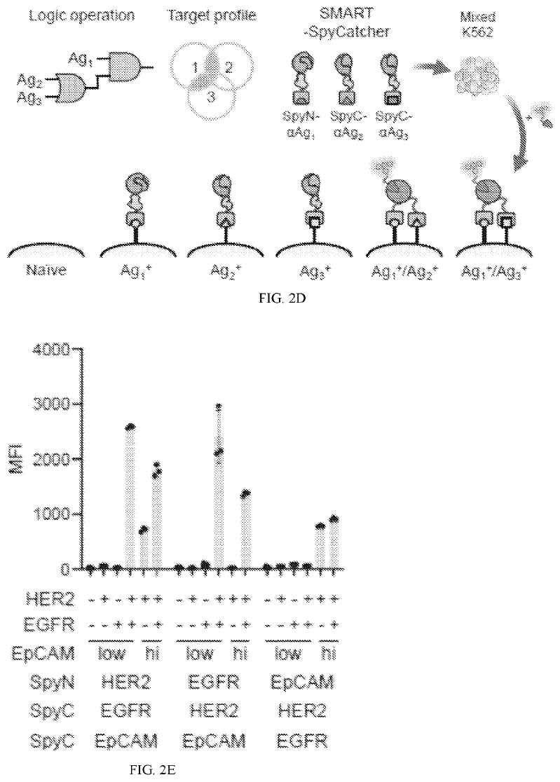

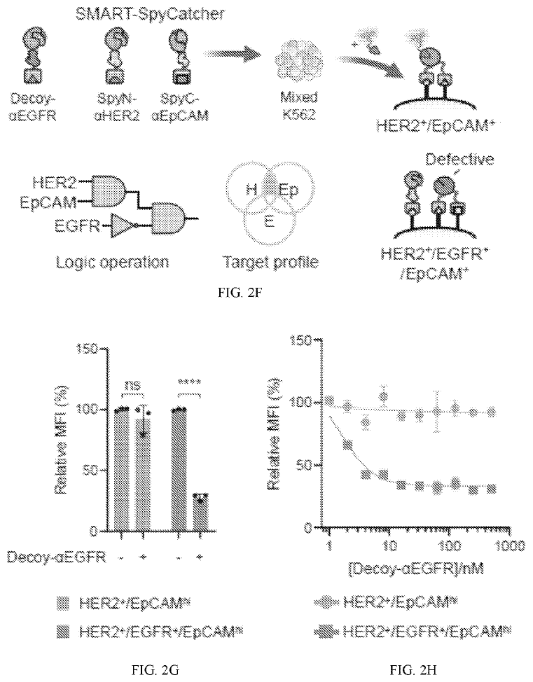

HER2 and EGFR engage in homo- and heteromultimeric surface clusters (FIG. 2A). In contrast, Epithelial Cell Adhesion Molecule (EpCAM) has not been reported to preferentially interact with either HER2 or EGFR, and actuation would therefore occur from chance encounters. When tested on mixed K562 cell lines expressing either low endogenous levels or induced to overexpress EpCAM in combination with different profiles of HER2 and EGFR, SMART-SpyCatcher computed AND functions on cells that fulfilled the assigned logic gate (FIG. 2B). SMART-SpyCatcher can therefore be used to target antigens that stochastically encounter each other or casually co-reside. SMART-SpyCatcher had a reduced activation profile on cells with low endogenous levels of EpCAM compared to these stably overexpressing the antigen, when employing AND gates involving the antigen. For instance, using SpyN-αHER2 and SpyC-αEpCAM for [HER2 AND EpCAM] logic resulted in a 15-fold difference in AF594 signal intensity between K562^{HER2+/EpCAMlo} and K562^{HER2+/EpCAMhi}. Yet, the level of actuation on either cell line in response to [HER2 AND EpCAM] logic was adjustable using tuned versions of eNrdJ-1N^{cage} (FIG. 2C).

### 3-Input AND/OR Logic

The SMART platform encompasses and can be used to implement OR operations (17A-17B). Furthermore, when combined with AND gates and utilizing 3 antigen inputs, the OR operator unlocks breadth in a targeting scheme. For instance, the [Ag₁ AND either Ag₂ OR Ag₃] logic uses one fixed and two variable antigens (FIG. 2D), where SMART targeting is constrained by the fixed antigen. This was illustrated by employing SpyN-αHER2 and SpyC-αEGFR/SpyC-αEpCAM, where the SMART-SpyCatcher functions by [HER2 AND either EGFR or EpCAM] logic, thereby targeting the three subpopulations K562^{HER2+/EGFR+/EpCAMlo}, K562^{HER2+/EpCAMhi} and K562^{HER2+/EGFR+/EpCAMhi} respectively 129-fold, 36-fold, and 89-fold above background, without responding to cells missing HER2 or both of EGFR and EpCAM (FIG. 2E). Therefore, combining AND/OR logic can be useful in heterogeneous cell communities where a single antigen is shared by target and non-target cell populations, while pairs of variable antigens can help define distinct subpopulations.

### 3-Input AND/NOT Logic

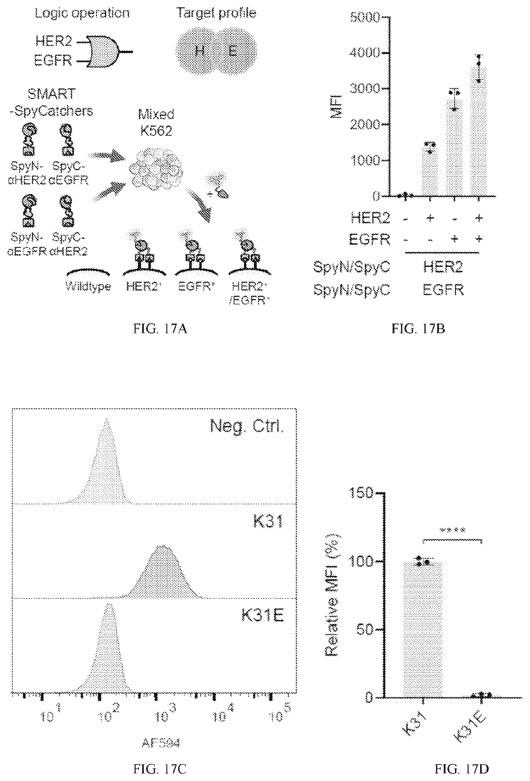

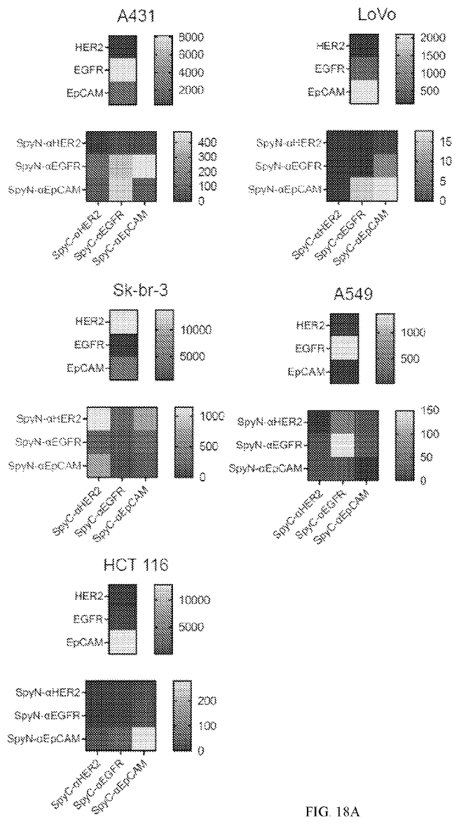

The lack of a surface marker between closely related cell populations could in principle be exploited through NOT gating for negative selection upon recognition. A NOT operator was implemented through a decoy that generates a defective POI upon actuation and outcompetes the reconstitution of the functional SpyCatcher output (FIG. 2F). That is, to make such decoy, SpyN was used as a template, with first introducing K31E into its N-terminal SpyCatcher003 fragment, thereby disabling the decoy's ability to capture SpyTag003 once the POI was reconstituted (FIG. 17C). The decoy's reactivity with SpyC was then enhanced by tapping into the tunability of its split intein actuator component; the cage of eNrdJ-1N^{cage} was manipulated by introducing K114AK116A and shortening its C-terminal from 35 to 29 amino acid residues. The decoy was then targeted at EGFR. Upon co-incubation of SpyN-αHER2, SpyC-αEpCAM, and Decoy-αEGFR with a mixture of K562 cell lines to assess [HER2 AND EpCAM NOT EGFR] logic, we observed that proper output associated with K562^{HER2+/EGFR+/EpCAMhi} was reduced by 72% whilst K562^{HER2+/EpCAMhi} was unaffected (FIG. 2G). Furthermore, dosing-in Decoy-αEGFR over a broad concentration range only minimally obscured actuation of SpyN-αHER2 and SpyC-αEpCAM on K562HER2/EpCAMhi cells, while K562HER2/EGFR/EpCAMhi cells experienced a decreased probe association (FIG. 2H).

### AND Gated Performance on Wildtype Cell Lines

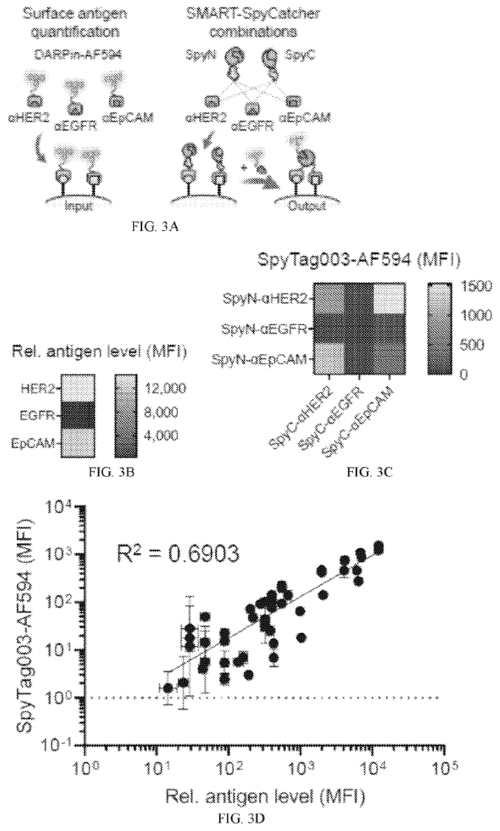

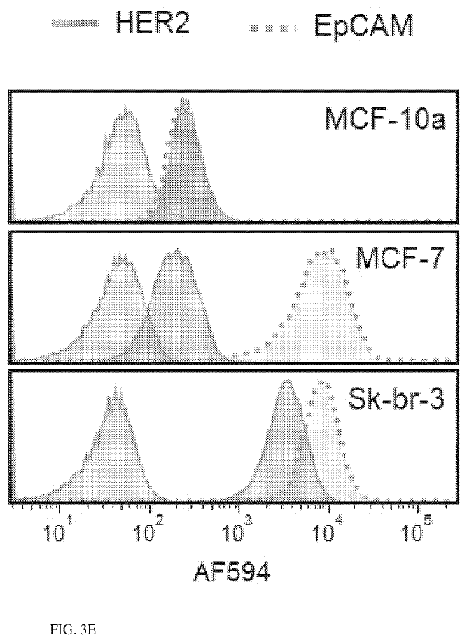

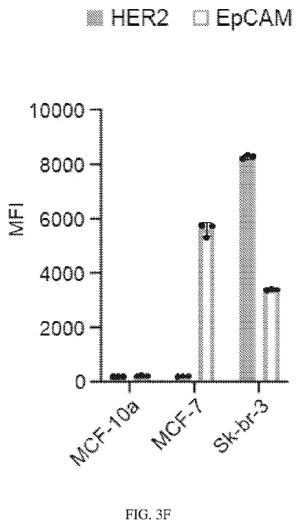

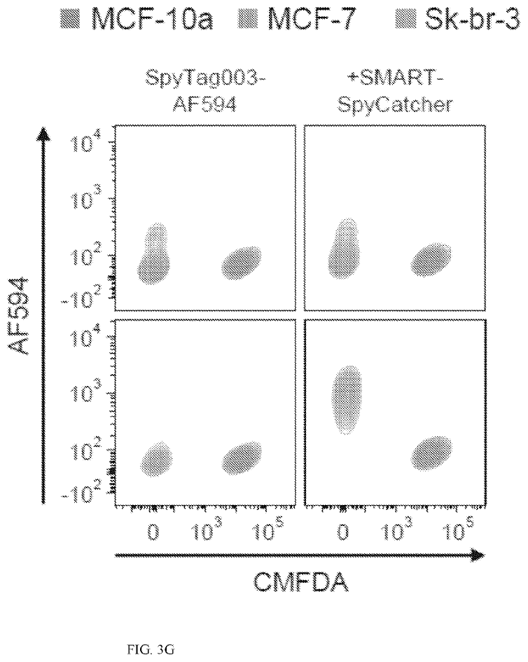

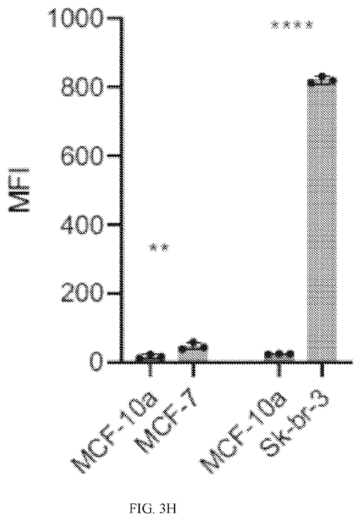

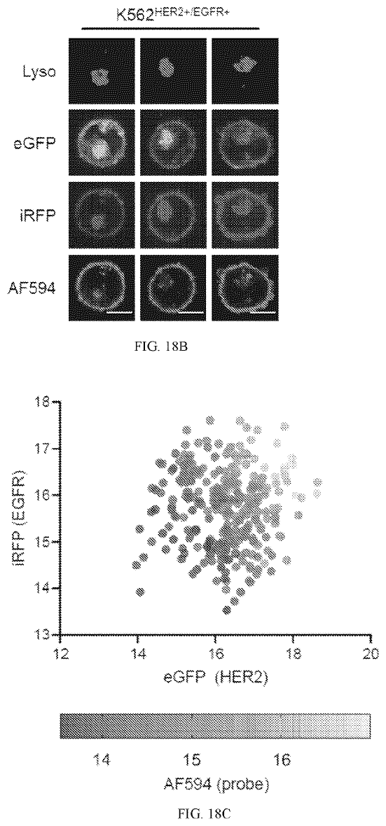

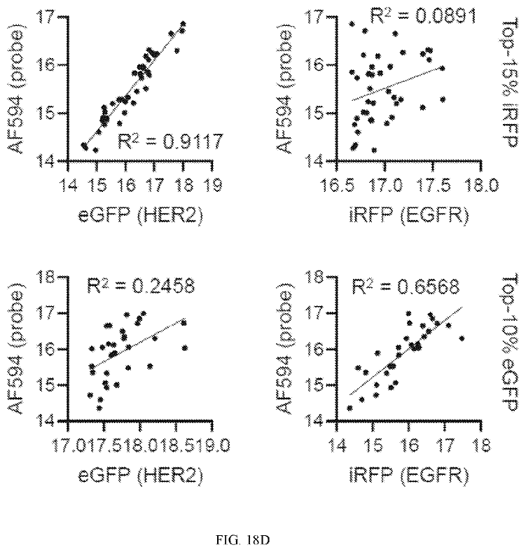

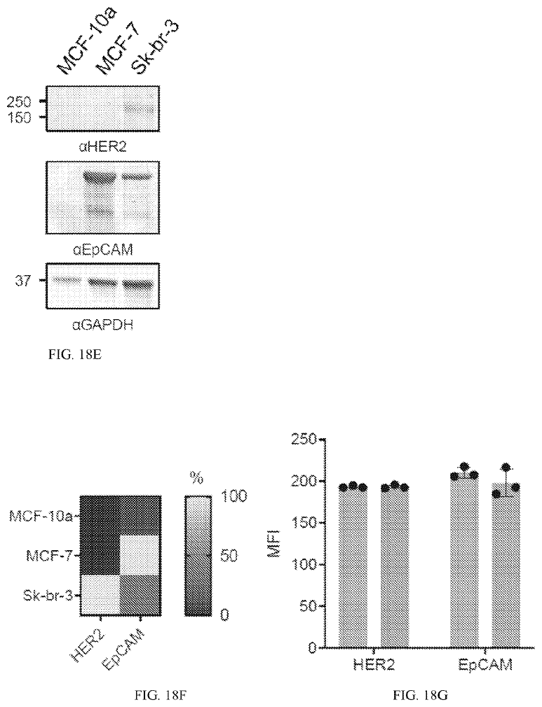

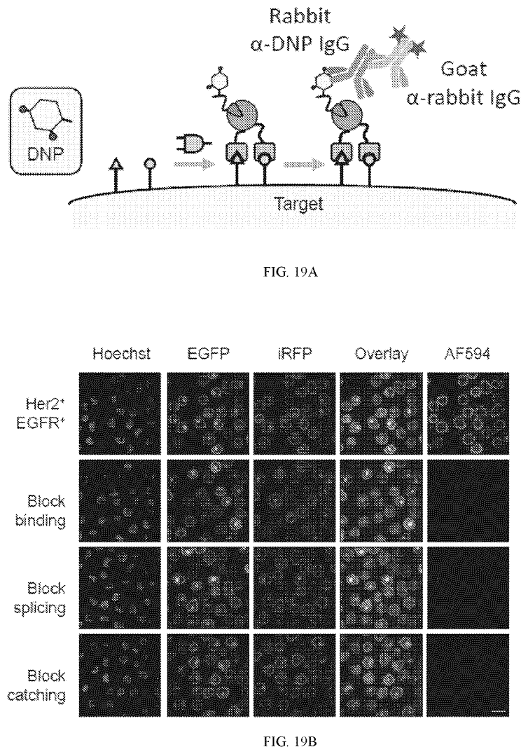

The Boolean reactivity of SMART-SpyCatcher to endogenous surface antigen landscapes was also evaluated on a variety of cancer cell lines using various combinations of SpyN and SpyC (FIG. 3A). First, DARPins labeled with AF594 were used to estimate the relative surface levels of HER2, EGFR, and EpCAM for each cell line (FIGS. 3A-3B, FIG. 18A). Then, the AND gated recruitment of SpyTag003-AF594 to the cells achieved by different sets of SpyNs and SpyCs was recorded (FIG. 3C, FIG. 18A). Plotting the median quantity of the lesser-expressed antigen used in each AND gate against the median recruitment of SpyTag003-AF594 achieved by the given logic operation gave a positive linear correlation accounting for 69% of the responsiveness of SMART-SpyCatcher (FIG. 3D). A similar tendency was observed with the K562^{HER2+/EGFR+} cell line (FIGS. 18B-18D). While this does not take cell morphology and membrane organization into consideration, this implies that the level of SMART actuation is largely dominated by the quantities and possibly the densities of the target antigens, where a threshold mechanism acts to protect cells with low or dilute surface levels. We further validated this observation on mixed human mammary cell lines displaying endogenous levels of HER2 and EpCAM (FIG. 3E-F and FIG. 18E-G), thereby mimicking the heterogenous environment of a diseased tissue. Two combinations (populations) of cells were tested: epithelial MCF-10a (HER2^{low}, EpCAM^{low}) was co-cultured with MCF-7 (HER2^{low}, EpCAM^{high}) as mixed population 1 or with Sk-br-3 (HER2^{high}, EpCAM^{high}) as mixed population 2. To enhance the recruitment of SpyTag003-AF594 we used SMART-SpyCatcher utilizing eNrdJ-1^{cage} with the additional K114AK119A mutations. Neither mixed population effectively recruited SpyTag003-AF594 on its own; however, once SpyN-αHER2 and SpyC-αEpCAM were added, recruitment occurred predominately for subpopulation Sk-br-3, with little-to-no probe associated with the other subpopulations MCF-10a or MCF-7 (FIG. 3G). When compared across the two populations the AF594 signal was only 2.3-fold higher for MCF-7 but 39-fold higher for Sk-br-3 over MCF10a (FIG. 3H). In summary, SMART-SpyCatcher can sense and react to endogenous surface antigen landscapes and use this ability to distinguish between health-like and cancerous subpopulations by Boolean logic. Additionally, cells with profiles that match the applied logic function may be guarded from SMART activation if one or both antigens are represented at low levels.

### Selective Delivery of Functionalities

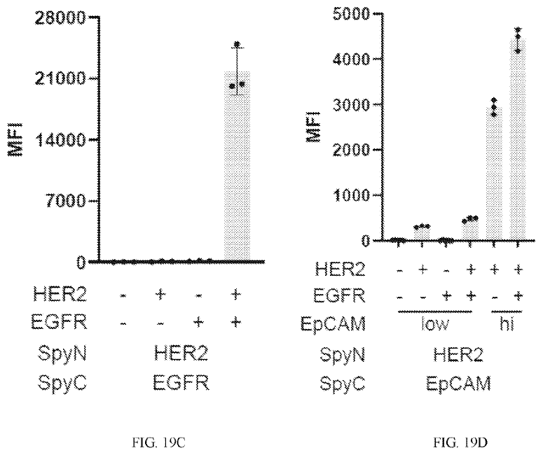

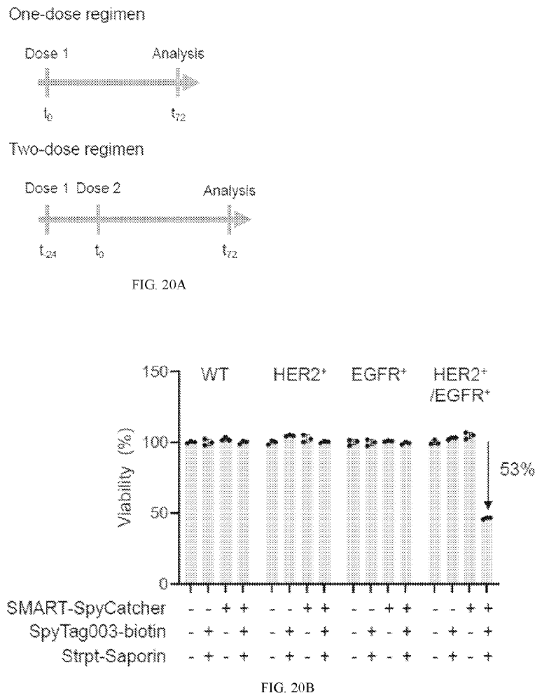

SMART-SpyCatcher generates a unique protein loading dock once activated cell-selectively, where the incoming peptide probe can be made with a wide array of functionalities to sculpt the cell surface. Of interest is the use of bifunctional small molecules to surface decorate a selected cell population for the recruitment of antibodies. We tested the use of SpyTag003 conjugated to dinitrophenol (SpyTag003-DNP) to recruit anti-DNP antibodies to K562^{HER2+/EGFR+} cells. Once incubated with SMART-SpyCatcher for [HER2 AND EGFR] logic and SpyTag003-DNP, we found the cells' surface decorated with the small molecule hapten as detected using a primary rabbit anti-DNP antibody and a secondary goat anti-rabbit AF594 conjugate (FIGS. 19A-19B). In a mixed K562 population setting, SMART-SpyCatcher successfully led to the recruitment of anti-DNP antibody to the targeted cell lines while avoiding engagement of non-target subpopulations (FIGS. 19C-19D).

### Depletion of Target Cell Line

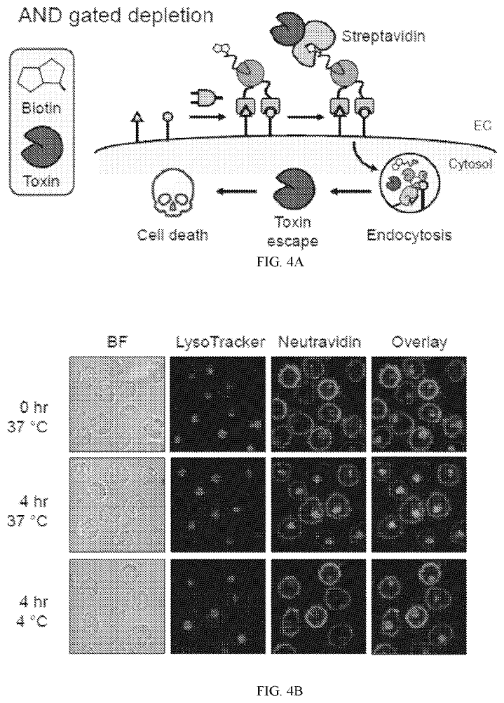

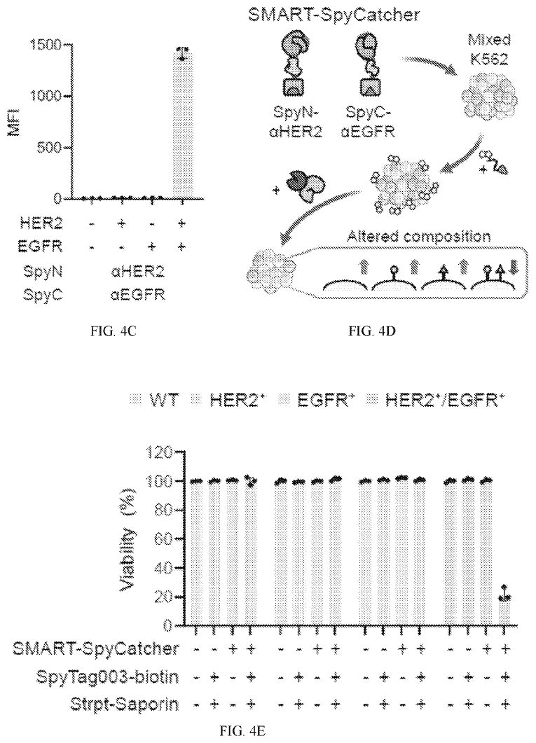

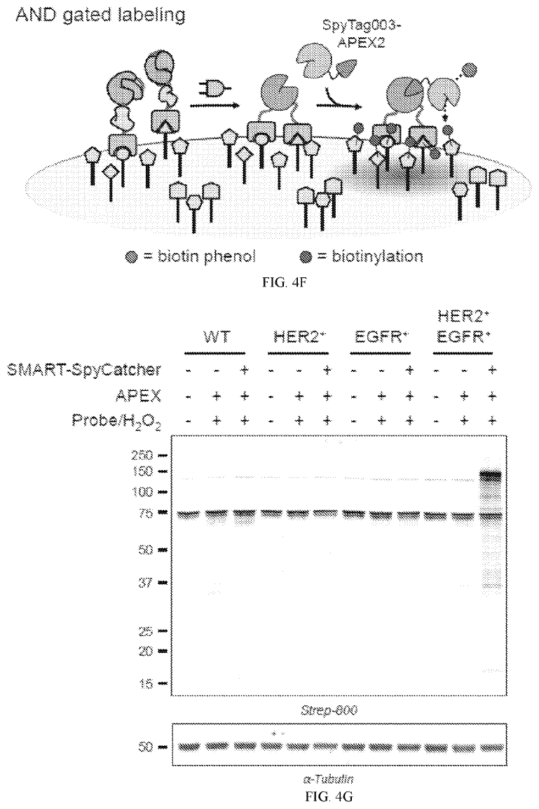

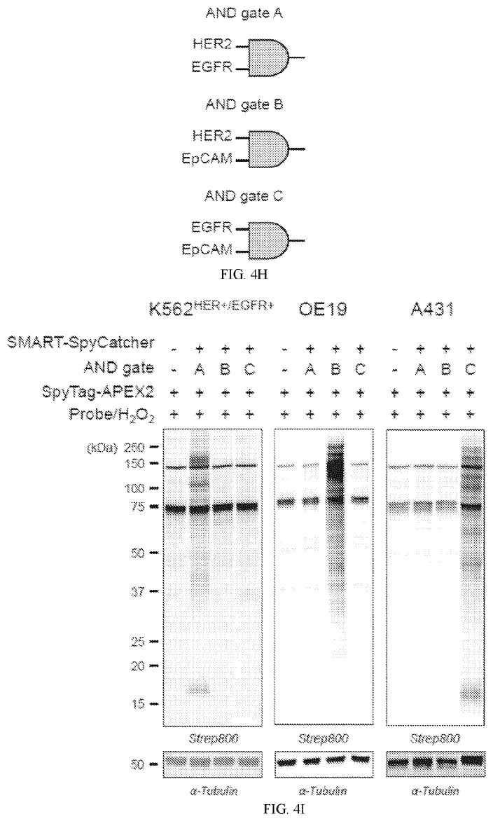

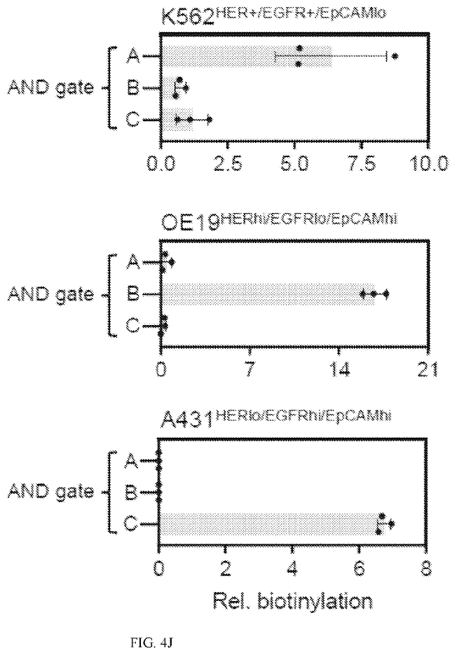

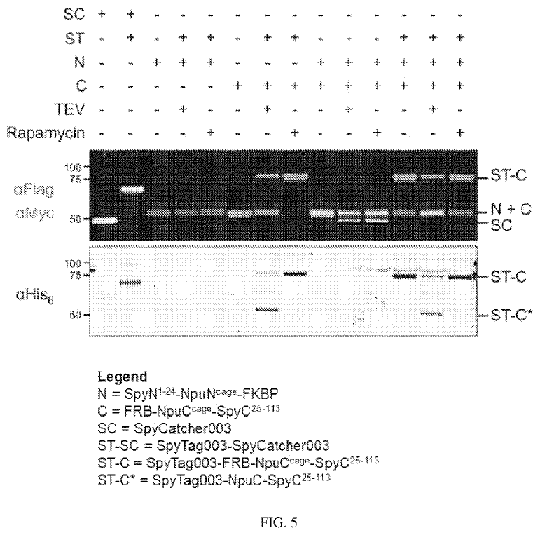

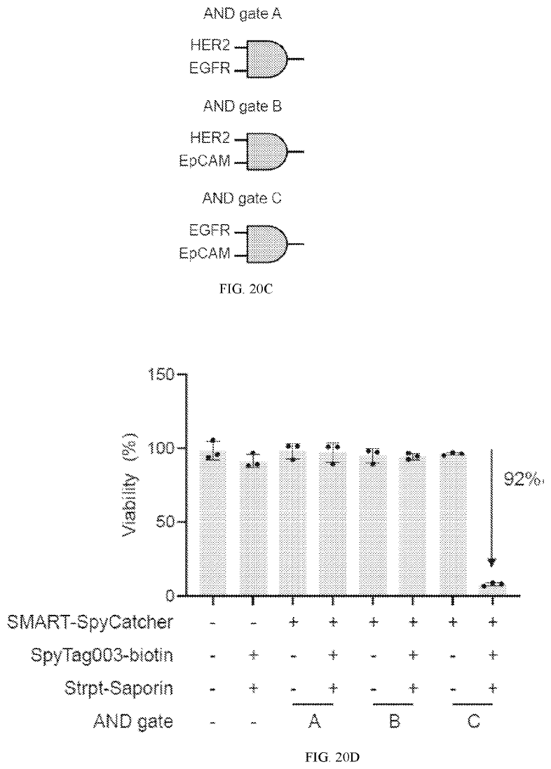

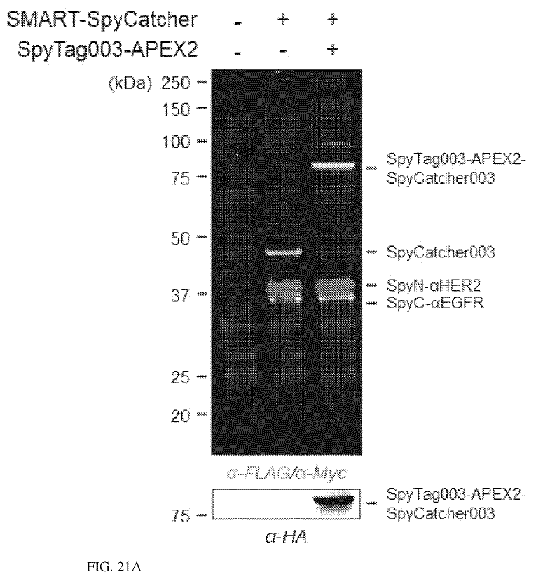

As an application, specific delivery and intake of a toxin can enable exclusive depletion of a target cell line in a heterogeneous population. While many ribosome-inactivating proteins can facilitate their own cellular intake, the plant toxin Saporin lacks a targeting domain to do so. Whenever internalized, however, Saporin depurinates ribosomes to induce translational arrest and cell death²⁴. A Streptavidin-Saporin conjugate was used to test for and demonstrate cell selective depletion (FIG. 4A). First, it was validated that AND gated CPS of SpyCatcher003 and delivery of SpyTag003-biotin recruited NeutrAvidin Rhodamine Red-X conjugate to K562^{HER2+EGFR+} cells and induced its internalization to endosomal/lysosomal compartments (FIG. 4B), and that this method was selective (FIG. 4C). Then the NeutrAvidin Rhodamine Red-X conjugate was replaced with a Streptavidin-Saporin conjugate in a similar setup. Mixed K562 cells were treated with SMART-SpyCatcher implementing [HER2 AND EGFR] logic and SpyTag003-biotin and subsequently cultured with Streptavidin-Saporin (FIG. 4D and FIG. 20A). After an additional 72 hours the cultures were sampled for variations in cell composition. While a one-dose regimen led to a 53% reduction of K562^{HER2+/EGFR+} cells (FIG. 20B), a two-dose regimen led to a 78% depletion of the subpopulation (FIG. 4E). Importantly, neither wildtype, K562^{HER2+}, nor K562^{EGFR+} cell lines were depleted to a similar extent in the co-culture following either the one- or two-dose regimens. Also tested was depletion of a A431 cell line, which displays low levels of HER2 but high levels of EGFR and EpCAM (FIG. 18A). For this, we used eNrdJ-1^{cage(K114AK116A)} to enhance recruitment of SpyTag003-biotin and various AND gates (FIG. 20C). Compared to an untreated control, A431 was depleted by 92% when using a two-dose regimen that employed SpyTag003-biotin, streptavidin-Saporin and SMART-SpyCatcher operating by [EGFR AND EpCAM] logic (FIG. 20D). Identical settings using SMART-SpyCatcher set up for [HER2 AND EGFR] or [HER2 AND EpCAM] logics failed to achieve comparable reductions and no reduction was observed when the toxin conjugate was omitted. Hence, SMART-SpyCatcher unlocks logic gated cell depletion strategies, which can be useful to study distinct groups of cells and to differently affect (e.g., through selective killing) distinct groups of cells.

That is, a biotinylated probe was used that could bind with the reporter protein conjugate NeutrAvidin Rhodamine Red-X conjugate (NA-Red), which is an imaging agent. Through binding and activation of the split-actuator system and subsequent probe capture clear colocalization of NA-Red on a (antigen) double positive cell surface was observed. Extended incubation at 37° C. prompted an internalization of the signal to lysosomal compartments, which was indicative of cargo uptake. This indicated that the reconstituted SpyCatcher003 enabled the delivery and internalization of protein-based cargo.

Imaging agents other than or in addition to Rhodamine Red can be used to image a target cell line in a heterogeneous population. For example, an imaging agent can be a dye, a protein, a radionuclide, a radiocontrast agent, or a magnetic contrast agent for magnetic resonance imaging (MRI). Imaging can be used, for example, to diagnose a hyperproliferative disorder, such as a benign tumor, malignant tumor, or cancer.

The split-actuator system was then again used in combination with a biotinylated probe but to deliver a Toxin-Streptavidin conjugate (disulfide linked). Post-endocytosis, the toxin can escape into the cytosol and perform its toxic activity. That is, the toxin Saporin depurinates ribosomal RNA, effectively halting protein synthesis; it is possible that Saporin also enters the nucleus and damages genomic DNA.

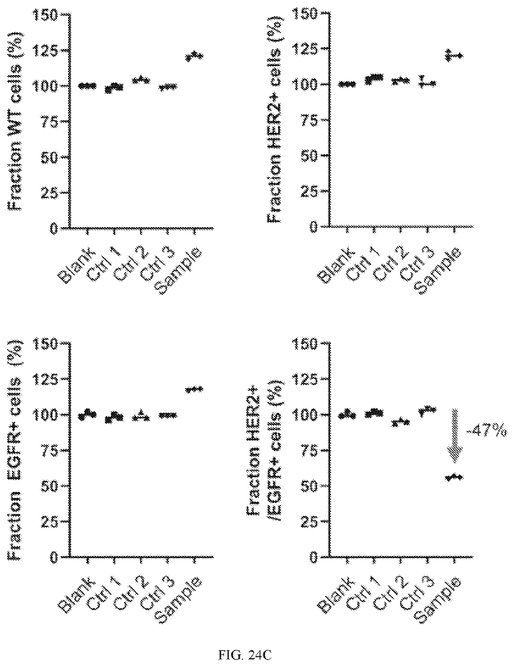

Using flow cytometry for analysis, a significant perturbation in cell line ratios between sample, controls, and a blank untreated control was observed. Complete single-dose treatment depleted as much as 47% of the (antigen) double positive cell line, without significantly affecting the other K562 variants. In contrast, leaving out any constituent (split-actuator system, biotinylated probe, Toxin-Streptavidin) resulted in a minimal effect on any cell line. Therefore, use of the split-actuator system to implement an AND gating strategy enabled killing of a certain cell line while avoiding harm to the other cell lines (FIGS. 24A-24C).

Toxins other than or in addition to Saporin can be used for specific delivery and/or intake of a toxin for exclusive depletion of a target cell line in a heterogeneous population. For example, a toxin can be a protein, a naturally-occurring protein, a modified naturally-occurring protein, a synthetic protein, an enzyme, a nucleotide, a nucleotide analog, a polynucleotide, a chemotherapeutic agent, a small-molecule chemotherapeutic agent, a metallo-organic chemotherapeutic agent, or a radionuclide. Depletion (and/or killing) of a target cell line can be used, for example, to treat a hyperproliferative disorder, such as a benign tumor, a malignant tumor, or cancer.

### Cell-Type Specific Delivery of APEX2

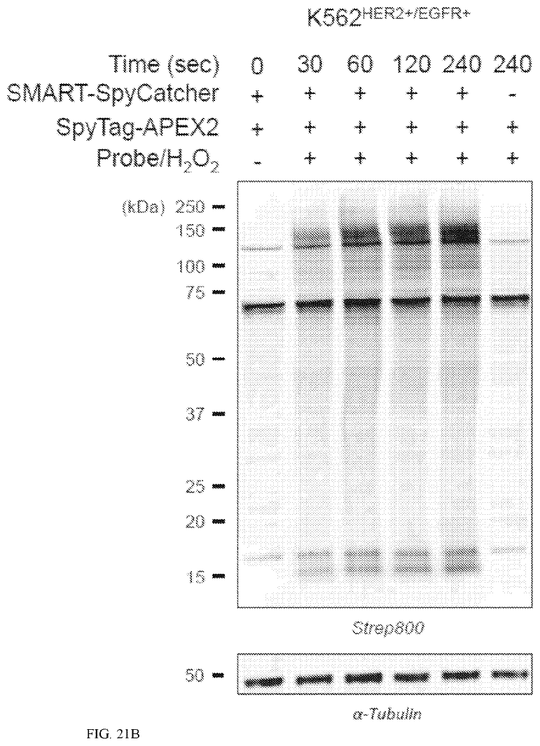

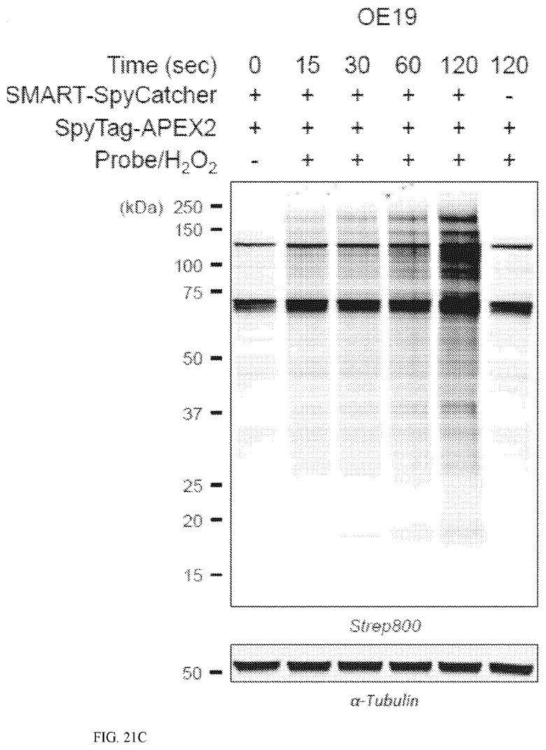

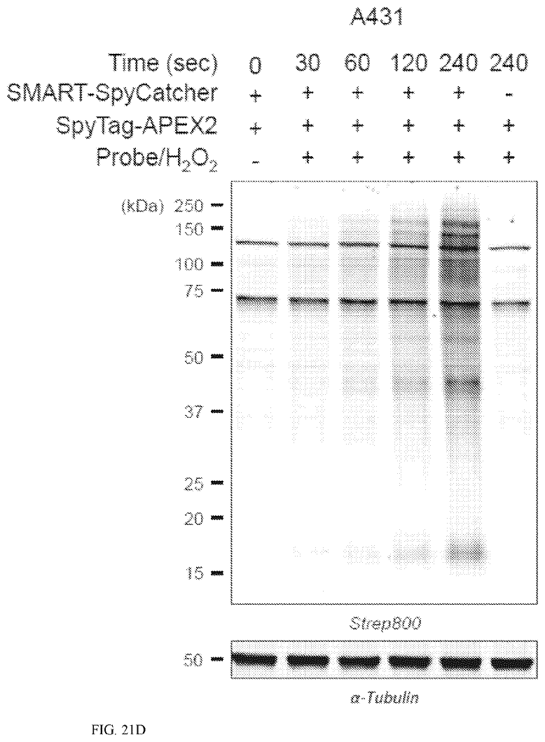

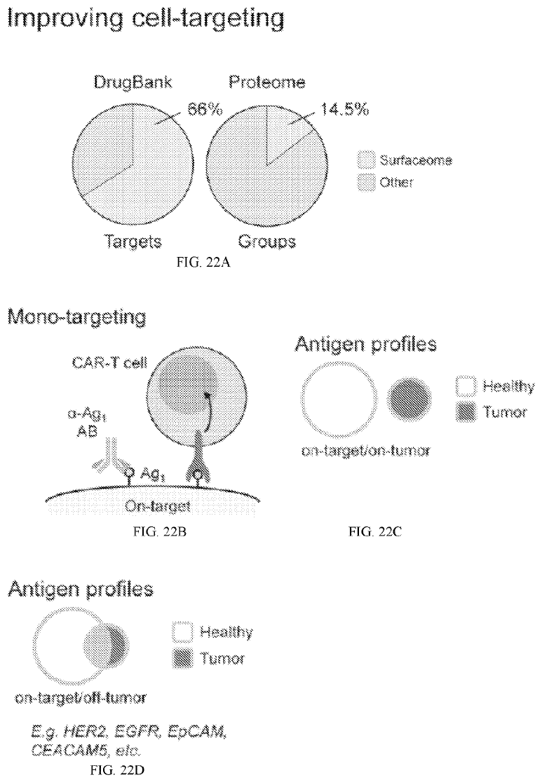

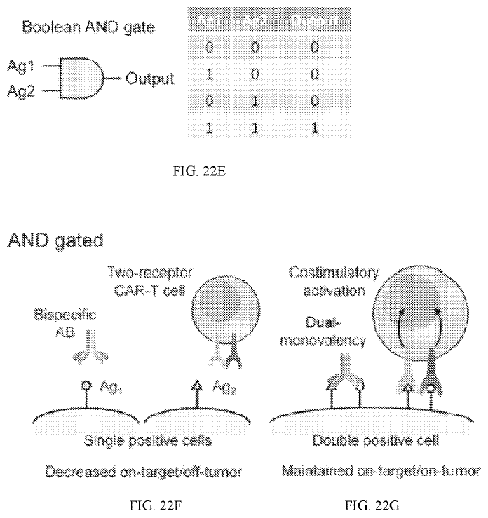

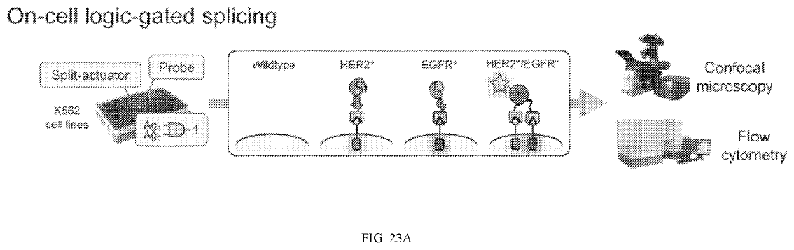

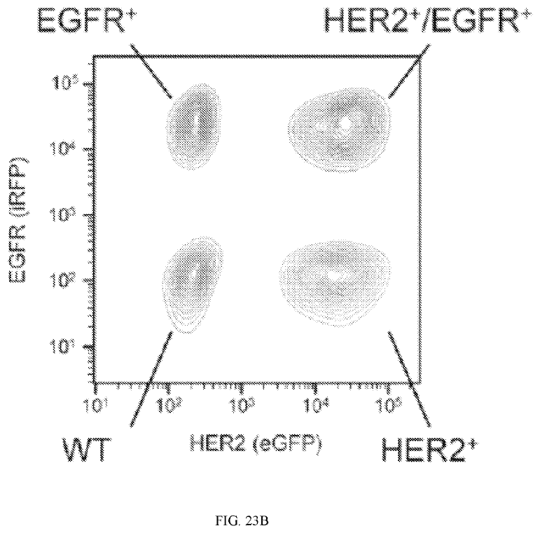

We also established an application for delivering APEX2²⁵ for cell selective proximity-labeling (FIG. 4F). Following incubation of K562^{HER2+EGFR+} with SMART-SpyCatcher for [HER2 AND EGFR] logic, reconstitution of SpyCatcher003, and delivery of SpyTag003-APEX2 (FIG. 21A) we performed a standard APEX2 labeling protocol with a biotin-phenol probe and activation upon addition of H₂O₂. APEX2 is useful for interactome mapping. Western blotting and detection using IRDye 800CW Streptavidin showed that K562^{HER2+/EGFR+} was biotinylated, a phenomenon that depended upon the AND gated activity of SMART-SpyCatcher (FIG. 4G). In comparison, single positive and wildtype K562 cells were not biotinylated under identical conditions (FIG. 4G). We validated these results further on K562^{HER2+/EGFR+} (HER2^{hi}, EGFR^{hi}, EpCAM^{1o}), OE19 (HER2^{hi}, EGFR^{lo}, EpCAM^{hi}), and A431 (HER2^{lo}, EGFR^{hi}, EpCAM^{hi}) while performing either [HER2 AND EGFR], [HER2 AND EpCAM], or [EGFR AND EpCAM] logic operations (FIG. 4H) prior to incubation with SpyTag003-APEX2 and biotin-phenol labeling. Only when SMART-SpyCatcher was matched with the correct antigen profile of the individual cell line did we observe labeling, a clear indicator of the usefulness of AND gated splicing for the localization of and, thus, labeling by SpyTag003-APEX2 (FIGS. 4I-4J. FIGS. 21B-21D). Therefore, SMART-SpyCatcher provides the ability to perform AND gated proximity-labeling to study and map the neighborhoods for two antigens.

The close relatedness between various tissues and their disease states is a key challenge in securing absolute precision targeting using surface markers. For example, few markers have been recognized as tumor specific, and population specific combinatorial sets of antigens can be considered to achieve specificity¹⁻³. Synthetic biology opens for systems that can operate by Boolean logic to realize multifaceted pattern recognition. For instance, next generation Chimeric Antigen-Receptor T (CAR T) Cells can compute multiple antigen inputs to trigger targeted cell killing with limited adverse effects on non-targets^{7,26}. Conversely, few protein-based systems have been developed with similar decision-making capabilities⁸⁻¹¹. Rewiring protein function to operate by Boolean logic in response to environmental stimuli is a complex task. Herein is described the use of CPS to generate a protein switch that processes Boolean AND, OR, and NOT operations at a single cell level to activate the switch's function. The SMART-SpyCatcher device, an embodiment of the present invention, enables selective cargo delivery to a target cell population. In addition, the SMART platform itself can be used to rewire the activity of any POT to make it operate through Boolean logic, thereby unlocking numerous applications.

### Protein Sequences

Below are listed the primary sequences (1-letter amino acid residue abbreviations used) for final protein products after recombinant expression and purification, along with overviews of the domain structures of these proteins.

TABLE 1A
Proteins used as probes or for delivery.
Protein primary structures from Table 1A (shown
with unprocessed N-terminal methionine and
His₆-SUMO tag when relevant):

| Protein identifier | Domain structure |
|---|---|
| SpyTag003-Cys | His₆-SpyTag003-SUMO-Cys |
| SpyTag003^{D117A}-Cys | His₆-SpyTag003^{D117A}-SUMO-Cys |
| SpyTag003-APEX2 | His₆-SpyTag003-HA-APEX2 |
| αHER2-Cys DARPin | αHER2-HA-Cys |
| αEGFR-Cys DARPin | αEGFR-HA-Cys |
| αEpCAM-Cys DARPin | αEpCAM-HA-Cys |

SpyTag003-Cys
[SEQ. ID NO. 1]
MGSSHHHHHHSSGLVPRGSRGVPHIVMVDAYKRYKGSGESGMSDSEVNQEAKPEVKP
EVKPETHINLKVSDGSSEIFFKIKKTTPLRRLMEAFAKRQGKEMDSLRFLYDGIRIQADQT
PEDLDMEDNDIIEAHREQIGC
SpyTag003^{D117A}-Cys
[SEQ. ID NO. 2]
MGSSHHHHHHSSGLVPRGSRGVPHIVMVAAYKRYKGSGESGMSDSEVNQEAKPEVKP
EVKPETHINLKVSDGSSEIFFKIKKTTPLRRLMEAFAKRQGKEMDSLRFLYDGIRIQADQT
PEDLDMEDNDIIEAHREQIGC
SpyTag003-APEX2
[SEQ. ID NO. 3]
MGSSHHHHHHSSGLVPRGSRGVPHIVMVDAYKRYKGSGESGYPYDVPDYASSGGKSYP
TVSADYQDAVEKAKKKLRGFIAEKRCAPLMLRLAFHSAGTFDKGTKTGGPFGTIKHPAE
LAHSANNGLDIAVRLLEPLKAEFPILSYADFYQLAGVVAVEVTGGPKVPFHPGREDKPE
PPPEGRLPDPTKGSDHLRDVFGKAMGLTDQDIVALSGGHTIGAAHKERSGFEGPWTSNP
LIFDNSYFTELLSGEKEGLLQLPSDKALLSDPVFRPLVDKYAADEDAFFADYAEAHQKLS
ELGFADA
His₆-SUMO-αHER2-Cys DARPin
[SEQ. ID NO. 4]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DLGKKLLEAARAGQDDEVRILMANGADVNAKDEYGLTPLYLATAHGHLEIVEVLLKN
GADVNAVDAIGFTPLHLAAFIGHLEIAEVLLKHGADVNAQDKFGKTAFDISIGNGNEDL
AEILQKLNGGYPYDVPDYAGGSC
His₆-SUMO-αEGFR-Cys DARPin
[SEQ. ID NO. 5]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DLGKKLLEAARAGQDDEVRILMANGADVNAKDEYGLTPLYLATAHGHLEIVEVLLKN
GADVNAVDAIGFTPLHLAAFIGHLEIAEVLLKHGADVNAQDKFGKTAFDISIGNGNEDL
AEILQKLNGGYPYDVPDYAGGSC
His₆-SUMO-αEpCAM-Cys DARPin
[SEQ. ID NO. 6]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DLGKKLLEAARAGQDDEVRILVANGADVNAYFGTTPLHLAAAHGRLEIVEVLLKNGAD
VNAQDVWGITPLHLAAYNGHLEIVEVLLKYGADVNAHDTRGWTPLHLAAINGHLEIVE
VLLKNVADVNAQDRSGKTPFDLAIDNGNEDIAEVLQKAAKLNGGYPYDVPDYAGGSC

TABLE 1B
Proteins used to screen for optimal SpyCatcher003 split site
to generate SMART-SpyCatcher. A linker with two TEV cut sites
(SGGENLYFQGENLYFQGGSGG [SEQ. ID NO. 7]) was positioned
in between the caged split intein fragments and their cages.

| Protein identifier | Domain structure |
|---|---|
| SpyN¹⁻²⁴-NpuN^{cage}-FKBP | FLAG-αHER2-SpyN¹⁻²⁴-NpuN^{cage}-FKBP |
| SpyN¹⁻⁴²-NpuN^{cage}-FKBP | FLAG-αHER2-SpyN¹⁻⁴²-NpuN^{cage}-FKBP |
| SpyN¹⁻⁵⁵-NpuN^{cage}-FKBP | FLAG-αHER2-SpyN¹⁻⁵⁵-NpuN^{cage}-FKBP |
| SpyN¹⁻⁷³-NpuN^{cage}-FKBP | FLAG-αHER2-SpyN¹⁻⁷³-NpuN^{cage}-FKBP |
| SpyN¹⁻⁸²-NpuN^{cage}-FKBP | FLAG-αHER2-SpyN¹⁻⁸²-NpuN^{cage}-FKBP |
| SpyN¹⁻⁹⁰-NpuN^{cage}-FKBP | FLAG-αHER2-SpyN¹⁻⁹⁰-NpuN^{cage}-FKBP |
| FRB-NpuC^{cage}-SpyC²⁵⁻¹¹³ | FRB-NpuC^{cage}-SpyC²⁵⁻¹¹³-αEGFR-Myc |
| FRB-NpuC^{cage}-SpyC⁴³⁻¹¹³ | FRB-NpuC^{cage}-SpyC⁴³⁻¹¹³-αEGFR-Myc |
| FRB-NpuC^{cage}-SpyC⁵⁶⁻¹¹³ | FRB-NpuC^{cage}-SpyC⁵⁶⁻¹¹³-αEGFR-Myc |
| FRB-NpuC^{cage}-SpyC⁷⁴⁻¹¹³ | FRB-NpuC^{cage}-SpyC⁷⁴⁻¹¹³-αEGFR-Myc |
| FRB-NpuC^{cage}-SpyC⁸³⁻¹¹³ | FRB-NpuC^{cage}-SpyC⁸³⁻¹¹³-αEGFR-Myc |
| FRB-NpuC^{cage}-SpyC^{91 113} | FRB-NpuC^{cage}-SpyC⁹¹⁻¹¹³-αEGFR-Myc |

Protein primary structures from Supplementary Table 1B (shown with N-terminal His⁶-SUMO tag):

His₆-SUMO-SpyN¹⁻²⁴-NpuN^{cage}-FKBP
[SEQ. ID NO. 8]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSACLSYETEILTVEYGL
LPIGKIVEKRIECTVYSVDNNGNIYTQPVAQWHDRGKQKVFEYCLEDGSLIRATKDHKF
MTVDGQMLPIKEIFRRKLDLMRVDNLPNGSGGENLYFQGENLYFQGGSGGIEIATEKYL
GEQNVYDIGVERDHNFALKNGGYFQGIEIATEKYLGEQNVYDIGVERDHNFALKNGNS
AGGVQVETISPGDGRTFPKRGQTCVVHYTGMLEDGKKFDSSRDRNKPFKFMLGKQEVI
RGWEEGVAQMSVGQRAKLTISPDYAYGATGHPGIIPPHATLVFDVELLKLE
His₆-SUMO-SpyN¹⁻⁴²-NpuN^{cage}-FKBP
[SEQ. ID NO. 9]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGACLSYETEILTVEYGLLPIGKIVEKRIECTVYSVDNNGNIYTQPVAQWHDRGKQKVF
EYCLEDGSLIRATKDHKFMTVDGQMLPIKEIFRRKLDLMRVDNLPNGSGGENLYFQGEN
LYFQGGSGGIEIATEKYLGEQNVYDIGVERDHNFALKNGGYFQGIEIATEKYLGEQNVY
DIGVERDHNFALKNGNSAGGVQVETISPGDGRTFPKRGQTCVVHYTGMLEDGKKFDSS
RDRNKPFKFMLGKQEVIRGWEEGVAQMSVGQRAKLTISPDYAYGATGHPGIIPPHATLV
FDVELLKLE
His₆-SUMO-SpyN¹⁻³⁵-NpuN^{cage}-FKBP
[SEQ. ID NO. 10]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISCLSYETEILTVEYGLLPIGKIVEKRIECTVYSVDNNGNIYTQPV
AQWHDRGKQKVFEYCLEDGSLIRATKDHKFMTVDGQMLPIKEIFRRKLDLMRVDNLPN
GSGGENLYFQGENLYFQGGSGGIEIATEKYLGEQNVYDIGVERDHNFALKNGGYFQGIEI
ATEKYLGEQNVYDIGVERDHNFALKNGNSAGGVQVETISPGDGRTFPKRGQTCVVHYT
GMLEDGKKFDSSRDRNKPFKFMLGKQEVIRGWEEGVAQMSVGQRAKLTISPDYAYGA
TGHPGIIPPHATLVFDVELLKLE
His₆-SUMO-SpyN¹⁻⁷³-NpuN^{cage}-FKBP
[SEQ. ID NO. 11]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLSYETEILTVEYGLLPIGKIVEKRI
ECTVYSVDNNGNIYTQPVAQWHDRGKQKVFEYCLEDGSLIRATKDHKFMTVDGQMLPI
KEIFRRKLDLMRVDNLPNGSGGENLYFQGENLYFQGGSGGIEIATEKYLGEQNVYDIGV
ERDHNFALKNGGYFQGIEIATEKYLGEQNVYDIGVERDHNFALKNGNSAGGVQVETISP
GDGRTFPKRGQTCVVHYTGMLEDGKKFDSSRDRNKPFKFMLGKQEVIRGWEEGVAQM
SVGQRAKLTISPDYAYGATGHPGIIPPHATLVFDVELLKLE
His₆-SUMO-SpyN¹⁻⁸²-NpuN^{cage}-FKBP
[SEQ. ID NO. 12]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYTFVETAAPºCLSYETEILTVEYGLL
PIGKIVEKRIECTVYSVDNNGNIYTQPVAQWHDRGKQKVFEYCLEDGSLIRATKDHKFM
TVDGQMLPIKEIFRRKLDLMRVDNLPNGSGGENLYFQGENLYFQGGSGGIEIATEKYLG
EQNVYDIGVERDHNFALKNGGYFQGIEIATEKYLGEQNVYDIGVERDHNFALKNGNSA
GGVQVETISPGDGRTFPKRGQTCVVHYTGMLEDGKKFDSSRDRNKPFKFMLGKQEVIR
GWEEGVAQMSVGQRAKLTISPDYAYGATGHPGIIPPHATLVFDVELLKLE
His₆-SUMO-SpyN¹⁻⁹⁰-NpuN^{cage}-FKBP
[SEQ. ID NO. 13]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYTFVETAAPDGYEVATPICLSYETEI
LTVEYGLLPIGKIVEKRIECTVYSVDNNGNIYTQPVAQWHDRGKQKVFEYCLEDGSLIR
ATKDHKFMTVDGQMLPIKEIFRRKLDLMRVDNLPNGSGGENLYFQGENLYFQGGSGGI
EIATEKYLGEQNVYDIGVERDHNFALKNGGYFQGIEIATEKYLGEQNVYDIGVERDHNF
ALKNGNSAGGVQVETISPGDGRTFPKRGQTCVVHYTGMLEDGKKFDSSRDRNKPFKFM
LGKQEVIRGWEEGVAQMSVGQRAKLTISPDYAYGATGHPGIIPPHATLVEDVELLKLE
His₆-SUMO-FRB-NpuC^{cage}-SpyC²⁵⁻¹¹³
[SEQ. ID NO. 14]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
GRVAILWHEMWHEGLEEASRLYFGERNVKGMFEVLEPLHAMMERGPQTLKETSFNQA
YGRDLMEAQEWCRKYMKSGNVKDLLQAWDLYYHVFRRISGNGNGGEQEVFEYCLED
GSLIRATKDHKFMTVDGQMLPIDEIFERELDLMRVDNLPNGSGGENLYFQGENLYFQGG
SGGIKIATRKYLGKQNVYDIGVERDHNFALKNGFIASNCHIKFSKRDEDGRELAGATME
LRDSSGKTISTWISDGHVKDFYLYPGKYTFVETAAPDGYEVATPIEFTVNEDGQVTVDG
EATEGDAHTGSTSGSDLGKKLLEAARAGQDDEVRILMANGADVNADDTWGWTPLHLA
AYQGHLEIVEVLLKNGADVNAYDYIGWTPLHLAADGHLEIVEVLLKNGADVNASDYIG
DTPLHLAAHNGHLEIVEVLLKHGADVNAQDKFGKTAFDISIDNGNEDLAEILQKLNEQK
LISEEDL
His₆-SUMO-FRB-NpuC^{cage}-SpyC⁴³-
[SEQ. ID NO. 15]
¹¹³MGSSHHHHHHGSGLVPRGSASMSDSEVNQEAKPEVKPEVKPETHINLKVSDGSSEIFF
KIKKTTPLRRLMEAFAKRQGKEMDSLRFLYDGIRIQADQTPEDLDMEDNDIIEAHREQIG
GGRVAILWHEMWHEGLEEASRLYFGERNVKGMFEVLEPLHAMMERGPQTLKETSENQ
AYGRDLMEAQEWCRKYMKSGNVKDLLQAWDLYYHVFRRISGNGNGGEQEVFEYCLE
DGSLIRATKDHKFMTVDGQMLPIDEIFERELDLMRVDNLPNGSGGENLYFQGENLYFQG
GSGGIKIATRKYLGKQNVYDIGVERDHNFALKNGFIASNCMELRDSSGKTISTWISDGHV
KDFYLYPGKYTFVETAAPDGYEVATPIEFTVNEDGQVTVDGEATEGDAHTGSTSGSDLG
KKLLEAARAGQDDEVRILMANGADVNADDTWGWTPLHLAAYQGHLEIVEVLLKNGA
DVNAYDYIGWTPLHLAADGHLEIVEVLLKNGADVNASDYIGDTPLHLAAHNGHLEIVE
VLLKHGADVNAQDKFGKTAFDISIDNGNEDLAEILQKLNEQKLISEEDL
His₆-SUMO-FRB-NpuC^{cage}-SpyC⁵⁶⁻¹¹³
[SEQ. ID NO. 16]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
GRVAILWHEMWHEGLEEASRLYFGERNVKGMFEVLEPLHAMMERGPQTLKETSFNQA
YGRDLMEAQEWCRKYMKSGNVKDLLQAWDLYYHVFRRISGNGNGGEQEVFEYCLED
GSLIRATKDHKFMTVDGQMLPIDEIFERELDLMRVDNLPNGSGGENLYFQGENLYFQGG
SGGIKIATRKYLGKQNVYDIGVERDHNFALKNGFIASNCWISDGHVKDFYLYPGKYTFV
ETAAPDGYEVATPIEFTVNEDGQVTVDGEATEGDAHTGSTSGSDLGKKLLEAARAGQD
DEVRILMANGADVNADDTWGWTPLHLAAYQGHLEIVEVLLKNGADVNAYDYIGWTPL
HLAADGHLEIVEVLLKNGADVNASDYIGDTPLHLAAHNGHLEIVEVLLKHGADVNAQD
KFGKTAFDISIDNGNEDLAEILQKLNEQKLISEEDL
His₆-SUMO-FRB-NpuC^{cage}-SpyC⁷⁴⁻¹¹³
[SEQ. ID NO. 17]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
GRVAILWHEMWHEGLEEASRLYFGERNVKGMFEVLEPLHAMMERGPQTLKETSFNQA
YGRDLMEAQEWCRKYMKSGNVKDLLQAWDLYYHVFRRISGNGNGGEQEVFEYCLED
GSLIRATKDHKFMTVDGQMLPIDEIFERELDLMRVDNLPNGSGGENLYFQGENLYFQGG
SGGIKIATRKYLGKQNVYDIGVERDHNFALKNGFIASNCFVETAAPDGYEVATPIEFTVN
EDGQVTVDGEATEGDAHTGSTSGSDLGKKLLEAARAGQDDEVRILMANGADVNADDT
WGWTPLHLAAYQGHLEIVEVLLKNGADVNAYDYIGWTPLHLAADGHLEIVEVLLKNG
ADVNASDYIGDTPLHLAAHNGHLEIVEVLLKHGADVNAQDKFGKTAFDISIDNGNEDLA
EILQKLNEQKLISEEDL
His₆-SUMO-FRB-NpuC^{cage}-SpyC⁸³⁻¹¹³
[SEQ. ID NO. 18]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
GRVAILWHEMWHEGLEEASRLYFGERNVKGMFEVLEPLHAMMERGPQTLKETSFNQA
YGRDLMEAQEWCRKYMKSGNVKDLLQAWDLYYHVFRRISGNGNGGEQEVFEYCLED
GSLIRATKDHKFMTVDGQMLPIDEIFERELDLMRVDNLPNGSGGENLYFQGENLYFQGG
SGGIKIATRKYLGKQNVYDIGVERDHNFALKNGFIASNCYEVATPIEFTVNEDGQVTVD
GEATEGDAHTGSTSGSDLGKKLLEAARAGQDDEVRILMANGADVNADDTWGWTPLHL
AAYQGHLEIVEVLLKNGADVNAYDYIGWTPLHLAADGHLEIVEVLLKNGADVNASDYI
GDTPLHLAAHNGHLEIVEVLLKHGADVNAQDKFGKTAFDISIDNGNEDLAEILQKLNEQ
KLISEEDL
His₆-SUMO-FRB-NpuC^{cage}-SpyC⁹¹⁻¹¹³
[SEQ. ID NO. 19]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
GRVAILWHEMWHEGLEEASRLYFGERNVKGMFEVLEPLHAMMERGPQTLKETSFNQA
YGRDLMEAQEWCRKYMKSGNVKDLLQAWDLYYHVFRRISGNGNGGEQEVFEYCLED
GSLIRATKDHKFMTVDGQMLPIDEIFERELDLMRVDNLPNGSGGENLYFQGENLYFQGG
SGGIKIATRKYLGKQNVYDIGVERDHNFALKNGFIASNCFTVNEDGQVTVDGEATEGDA
HTGSTSGSDLGKKLLEAARAGQDDEVRILMANGADVNADDTWGWTPLHLAAYQGHL
EIVEVLLKNGADVNAYDYIGWTPLHLAADGHLEIVEVLLKNGADVNASDYIGDTPLHL
AAHNGHLEIVEVLLKHGADVNAQDKFGKTAFDISIDNGNEDLAEILQKLNEQKLISEEDL

TABLE 1C
Proteins for optimizing SMART-SpyCatcher.

| Protein identifier | Domain structure |
|---|---|
| SpyN¹⁻⁷³-NrdJ-1N^{cage}-FKBP | FLAG-αHER2-SpyN¹⁻⁷³-NrdJ-1N^{cage}-FKBP |
| SpyN¹⁻⁷³-NrdJ-1N(C1A)^{cage}-FKBP | FLAG-αHER2-SpyN¹⁻⁷³-NrdJ-1N(C1A)^{cage}-FKBP |
| SpyN¹⁻⁷³-NrdJ-1N(C76V)^{cage}-FKBP | FLAG-αHER2-SpyN¹⁻⁷³-NrdJ-1N(C76V)^{cage}-FKBP |
| FRB-NrdJ-1C^{cage}-SpyC⁷⁴⁻¹¹³ | FRB-NrdJ-1C^{cage}-SpyC⁷⁴⁻¹¹³-αEGFR-Myc |
| FRB-NrdJ-1C^{cage(C76V)}-SpyC⁷⁴⁻¹¹³ | FRB-NrdJ-1C^{cage(C76V)}-SpyC⁷⁴⁻¹¹³-αEGFR-Myc |

Protein primary structures from Table 1C (shown with N-terminal His⁶-SUMO tag):

His₆-SUMO-SpyN¹⁻⁷³-NrdJ-1N^{cage}-FKBP
[SEQ. ID NO. 20]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRCTPDHPVYTKN
RGYVRADELTDDDELVVAIENLYGDEVDGYFQGMEAKTYIGKLKSRKIVSNEDTYDIQT
STHNFFANDNSAGGVQVETISPGDGRTFPKRGQTCVVHYTGMLEDGKKFDSSRDRNKP
FKFMLGKQEVIRGWEEGVAQMSVGQRAKLTISPDYAYGATGHPGIIPPHATLVFDVELL
KLE
His₆-SUMO-SpyN¹⁻⁷³-NrdJ-1N(C1A)^{cage}-FKBP
[SEQ. ID NO. 21]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYALVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVDGYFQGMEAKTYIGKLKSRKIVSNEDTYDIQT
STHNFFANDNSAGGVQVETISPGDGRTFPKRGQTCVVHYTGMLEDGKKFDSSRDRNKP
FKFMLGKQEVIRGWEEGVAQMSVGQRAKLTISPDYAYGATGHPGIIPPHATLVFDVELL
KLE
His₆-SUMO-SpyN¹⁻⁷³-NrdJ-1N(C76V)^{cage}-FKBP
[SEQ. ID NO. 22]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVDGYFQGMEAKTYIGKLKSRKIVSNEDTYDIQT
STHNFFANDNSAGGVQVETISPGDGRTFPKRGQTCVVHYTGMLEDGKKFDSSRDRNKP
FKFMLGKQEVIRGWEEGVAQMSVGQRAKLTISPDYAYGATGHPGIIPPHATLVFDVELL
KLE
His₆-SUMO-FRB-NrdJ-1C^{cage}-SpyC⁷⁴⁻¹¹³
[SEQ. ID NO. 23]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
GRVAILWHEMWHEGLEEASRLYFGERNVKGMFEVLEPLHAMMERGPQTLKETSFNQA
YGRDLMEAQEWCRKYMKSGNVKDLLQAWDLYYHVFRRISGNGNGNAELVELEIDTLH
GVKTIRCTPDHPVYTKNRGYVRADELTDDDELVVAIENLYGDEVDGYFQGMEAKTYIG
KLKSRKIVSNEDTYDIQTSTHNFFANDILVHNSFVETAAPDGYEVATPIEFTVNEDGQVT
VDGEATEGDAHTGSTSGSDLGKKLLEAARAGQDDEVRILMANGADVNADDTWGWTP
LHLAAYQGHLEIVEVLLKNGADVNAYDYIGWTPLHLAADGHLEIVEVLLKNGADVNAS
DYIGDTPLHLAAHNGHLEIVEVLLKHGADVNAQDKFGKTAFDISIDNGNEDLAEILQKL
NEQKLISEEDL
His₆-SUMO-FRB-NrdJ-1C^{cage(C76V)}-SpyC⁷⁴-
[SEQ. ID NO. 24]
¹¹³MGSSHHHHHHGSGLVPRGSASMSDSEVNQEAKPEVKPEVKPETHINLKVSDGSSEIFF
KIKKTTPLRRLMEAFAKRQGKEMDSLRFLYDGIRIQADQTPEDLDMEDNDIIEAHREQIG
GGRVAILWHEMWHEGLEEASRLYFGERNVKGMFEVLEPLHAMMERGPQTLKETSFNQ
AYGRDLMEAQEWCRKYMKSGNVKDLLQAWDLYYHVFRRISGNGNGNAELVELEIDTL
HGVKTIRVTPDHPVYTKNRGYVRADELTDDDELVVAIENLYGDEVDGYFQGMEAKTYI
GKLKSRKIVSNEDTYDIQTSTHNFFANDILVHNSFVETAAPDGYEVATPIEFTVNEDGQV
TVDGEATEGDAHTGSTSGSDLGKKLLEAARAGQDDEVRILMANGADVNADDTWGWT
PLHLAAYQGHLEIVEVLLKNGADVNAYDYIGWTPLHLAADGHLEIVEVLLKNGADVNA
SDYIGDTPLHLAAHNGHLEIVEVLLKHGADVNAQDKFGKTAFDISIDNGNEDLAEILQKL
NEQKLISEEDL

TABLE 1D
Proteins used as standard SMART-SpyCatcher components.

| Protein identifier | Domain structure |
|---|---|
| αHER2-SpyN-NrdJ-1N^{cage(C76V)} | FLAG-αHER2-SpyN¹⁻⁷³-NrdJ-1N(C76V)^{cage} |
| αHER2-SpyN-eNrdJ-1N^{cage} | FLAG-αHER2-SpyN¹⁻⁷³-NrdJ-1N(C76V)^{cage(K104EK119A)} |
| αEGFR-SpyN-eNrdJ-1N^{cage} | FLAG-αEGFR-SpyN¹⁻⁷³-NrdJ-1N(C76V)^{cage(K104EK119A)} |
| αEpCAM-SpyN-eNrdJ-1N^{cage} | FLAG-αEpCAM-SpyN¹⁻⁷³-NrdJ-1N(C76V)^{cage(K104EK119A)} |
| NrdJ-1C^{cage(C76V)}-SpyC-αEGFR | NrdJ-1C^{cage(C76V)}-SpyC⁷⁴⁻¹¹³-αEGFR-Myc |
| eNrdJ-1C^{cage}-SpyC-αEGFR | NrdJ-1C^{cage(D66KC76V)}-SpyC⁷⁴⁻¹¹³-αEGFR-Myc |
| eNrdJ-1C^{cage}-SpyC-αHER2 | NrdJ-1C^{cage(D66KC76V)}-SpyC⁷⁴⁻¹¹³-αHER2-Myc |
| eNrdJ-1C^{cage}-SpyC-αEpCAM | NrdJ-1C^{cage(D66KC76V)}-SpyC⁷⁴⁻¹¹³-αEpCAM-Myc |

Protein primary structures from Supplementary Table 1D (shown with N-terminal His₆-SUMO tag):

His₆-SUMO-αHER2-SpyN-NrdJ-1N(C76V)^{cage}
[SEQ. ID NO. 25]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVDGYFQGMEAKTYIGKLKSRKIVSNEDTYDIQT
STHNFFAND
His₆-SUMO-αHER2-SpyN-eNrdJ-1N^{cage}
[SEQ. ID NO. 26]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFFAND
His₆-SUMO-αEGFR-SpyN-eNrdJ-
[SEQ. ID NO. 27]
INcageMGSSHHHHHHGSGLVPRGSASMSDSEVNQEAKPEVKPEVKPETHINLKVSDGSSE
IFFKIKKTTPLRRLMEAFAKRQGKEMDSLRFLYDGIRIQADQTPEDLDMEDNDIIEAHRE
QIGGDYKDDDDKDLGKKLLEAARAGQDDEVRILVANGADVNAYFGTTPLHLAAAHGR
LEIVEVLLKNGADVNAQDVWGITPLHLAAYNGHLEIVEVLLKYGADVNAHDTRGWTPL
HLAAINGHLEIVEVLLKNVADVNAQDRSGKTPFDLAIDNGNEDIAEVLQKAAKLNGSTS
GSVTTLSGLSGEQGPSGDMTTEEDSATHIKFSKRDEDGRELAGATMELRDSSGKTISTWI
SDGHVKDFYLYPGKYCLVGSSEIITRNYGKTTIKEVVEIFDNDKNIQVLAFNTHTDNIEW
APIKAAQLTRPNAELVELEIDTLHGVKTIRVTPDHPVYTKNRGYVRADELTDDDELVVAI
ENLYGDEVNGYFQGMEAETYIGKLKSRAIVSNEDTYDIQTSTHNFFAND
His₆-SUMO-αEpCAM-SpyN-eNrdJ-1N^{cage}
[SEQ. ID NO. 28]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILVANGADVNAYFGTTPLHLAAAHGRLEIV
EVLLKNGADVNAQDVWGITPLHLAAYNGHLEIVEVLLKYGADVNAHDTRGWTPLHLA
AINGHLEIVEVLLKNVADVNAQDRSGKTPFDLAIDNGNEDIAEVLQKAAKLNGSTSGSV
TTLSGLSGEQGPSGDMTTEEDSATHIKFSKRDEDGRELAGATMELRDSSGKTISTWISDG
HVKDFYLYPGKYCLVGSSEIITRNYGKTTIKEVVEIFDNDKNIQVLAFNTHTDNIEWAPIK
AAQLTRPNAELVELEIDTLHGVKTIRVTPDHPVYTKNRGYVRADELTDDDELVVAIENL
YGDEVNGYFQGMEAETYIGKLKSRAIVSNEDTYDIQTSTHNFFAND
His₆-SUMO--NrdJ-1C^{cage(C76V)}-SpyC-αEGFR
[SEQ. ID NO. 29]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
NAELVELEIDTLHGVKTIRVTPDHPVYTKNRGYVRADELTDDDELVVAIENLYGDEVDG
YFQGMEAKTYIGKLKSRKIVSNEDTYDIQTSTHNFFANDILVHNSFVETAAPDGYEVATP
IEFTVNEDGQVTVDGEATEGDAHTGSTSGSDLGKKLLEAARAGQDDEVRILMANGADV
NADDTWGWTPLHLAAYQGHLEIVEVLLKNGADVNAYDYIGWTPLHLAADGHLEIVEV
LLKNGADVNASDYIGDTPLHLAAHNGHLEIVEVLLKHGADVNAQDKFGKTAFDISIDNG
NEDLAEILQKLNEQKLISEEDL
His₆-SUMO-NrdJ-1C^{cage(C76V)}-SpyC-αEGFR
[SEQ. ID NO. 30]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
NAELVELEIKTLHGVKTIRVTPDHPVYTKNRGYVRADELTDDDELVVAIENLYGDEVNG
YFQGMEAKTYIGKLKSRKIVSNEDTYDIQTSTHNFFANDILVHNSFVETAAPDGYEVATP
IEFTVNEDGQVTVDGEATEGDAHTGSTSGSDLGKKLLEAARAGQDDEVRILMANGADV
NADDTWGWTPLHLAAYQGHLEIVEVLLKNGADVNAYDYIGWTPLHLAADGHLEIVEV
LLKNGADVNASDYIGDTPLHLAAHNGHLEIVEVLLKHGADVNAQDKFGKTAFDISIDNG
NEDLAEILQKLNEQKLISEEDL
His₆-SUMO-eNrdJ-1C^{cage}-SpyC-αHER2
[SEQ. ID NO. 31]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
NAELVELEIKTLHGVKTIRVTPDHPVYTKNRGYVRADELTDDDELVVAIENLYGDEVNG
YFQGMEAKTYIGKLKSRKIVSNEDTYDIQTSTHNFFANDILVHNSFVETAAPDGYEVATP
IEFTVNEDGQVTVDGEATEGDAHTGSTSGSDLGKKLLEAARAGQDDEVRILMANGADV
NAKDEYGLTPLYLATAHGHLEIVEVLLKNGADVNAVDAI
His₆-SUMO-eNrdJ-1C^{cage}-SpyC-αEpCAM
[SEQ. ID NO. 32]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
NAELVELEIKTLHGVKTIRVTPDHPVYTKNRGYVRADELTDDDELVVAIENLYGDEVNG
YFQGMEAKTYIGKLKSRKIVSNEDTYDIQTSTHNFFANDILVHNSFVETAAPDGYEVATP
IEFTVNEDGQVTVDGEATEGDAHTGSTSGSDLGKKLLEAARAGQDDEVRILVANGADV
NAYFGTTPLHLAAAHGRLEIVEVLLKNGADVNAQDVWGITPLHLAAYNGHLEIVEVLL
KYGADVNAHDTRGWTPLHLAAINGHLEIVEVLLKNVADVNAQDRSGKTPFDLAIDNGN
EDIAEVLQKAAKLNEQKLISEEDL

TABLE 1E
Proteins with tuned cage electrostatics.

| Protein identifier | Domain structure |
|---|---|
| αHER2-SpyN-eNrdJ- | FLAG-αHER2-SpyN¹⁻⁴²-NrdJ- |
| 1N^{cage(K114A)} | 1N(C76V)^{cage(K104EK114AK119A)} |
| αHER2-SpyN-eNrdJ- | FLAG-αHER2-SpyN¹⁻⁴²-NrdJ- |
| 1N^{cage(K116A)} | 1N(C76V)^{cage(K104EK116AK119A)} |
| αHER2-SpyN-eNrdJ- | FLAG-αHER2-SpyN¹⁻⁴²-NrdJ- |
| 1N^{cage(R118A)} | 1N(C76V)^{cage(K104ER118AK119A)} |
| αHER2-SpyN-eNrdJ- | FLAG-αHER2-SpyN¹⁻⁴²-NrdJ- |
| 1N^{cage(K114AK116A)} | 1N(C76V)^{cage(K104EK114AK116AK119A)} |
| αHER2-SpyN-eNrd.J- | FLAG-αHER2-SpyN¹⁻⁴²-NrdJ- |
| 1N^{cage(K114AK116AR118A)} | 1N(C76V)^{cage(K104EK114AK116AR118AK119A)} |
| αHER2-SpyN-eNrdJ- | FLAG-αHER2-SpyN¹⁻⁴²-NrdJ- |
| 1N^{cage(A119K)} | 1N(C76V)^{cage(K104E)} |
| αEGFR-SpyN-eNrdJ- | FLAG-αEGFR-SpyN¹⁻⁴²-NrdJ- |
| 1N^{cage(K114AK116A)} | 1N(C76V)^{cage(K104EK114AK116AK119A)} |
| αEpCAM-SpyN-eNrdJ- | FLAG-αEpCAM-SpyN¹⁻⁴²-NrdJ- |
| 1N^{cage(K114AK116A)} | 1N(C76V)^{cage(K104EK114AK116AK119A)} |

Protein primary structures from Supplementary Table XX (shown with N-terminal His₆-SUMO tag):

His₆-SUMO-αHER2-SpyN-eNrdJ-1N^{cage(K114A)}
[SEQ. ID NO. 33]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGALKSRAIVSNEDTYDIQT
STHNFFAND
His₆-SUMO-αHER2-SpyN-eNrdJ-1N^{cage(K116A)}
[SEQ. ID NO. 34]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLASRAIVSNEDTYDIQT
STHNFFAND
His₆-SUMO-αHER2-SpyN-eNrdJ-1N^{cage(R118A)}
[SEQ. ID NO. 35]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSAAIVSNEDTYDIQT
STHNFFAN
His₆-SUMO-αHER2-SpyN-eNrdJ-1N^{cage(K114AK116A)}
[SEQ. ID NO. 36]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGALASRAIVSNEDTYDIQT
STHNFFAND
His₆-SUMO-αHER2-SpyN-eNrdJ-1N^{cage(K114AK116AR118A)}
[SEQ. ID NO. 37]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGALASAAIVSNEDTYDIQT
STHNFFAND
His₆-SUMO-αHER2-SpyN-eNrdJ-1N^{cage(A119K)}
[SEQ. ID NO. 38]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRKIVSNEDTYDIQT
STHNFFAND
His₆-SUMO-αEGFR-SpyN-eNrdJ-1N^{cage(K114AK116A)}
[SEQ. ID NO. 39]
MGSSHHHHHHGSGLVPRGSASMSDSEVNQEAKPEVKPEVKPETHINLKVSDGSSEIFFKI
KKTTPLRLMEAFAKRQGKEMDSLRFLYDGIRIQADQTPEDLDMEDNDIIEAHREQIGG
DYKDDDDKDLGKKLLEAARAGQDDEVRILVANGADVNAYFGTTPLHLAAAHGRLEIV
EVLLKNGADVNAQDVWGITPLHLAAYNGHLEIVEVLLKYGADVNAHDTRGWTPLHLA
AINGHLEIVEVLLKNVADVNAQDRSGKTPFDLAIDNGNEDIAEVLQKAAKLNGSTSGSV
TTLSGLSGEQGPSGDMTTEEDSATHIKFSKRDEDGRELAGATMELRDSSGKTISTWISDG
HVKDFYLYPGKYCLVGSSEIITRNYGKTTIKEVVEIFDNDKNIQVLAFNTHTDNIEWAPIK
AAQLTRPNAELVELEIDTLHGVKTIRVTPDHPVYTKNRGYVRADELTDDDELVVAIENL
YGDEVNGYFQGMEAETYIGALASRAIVSNEDTYDIQTSTHNFFAND
His₆-SUMO-αEpCAM-SpyN-eNrdJ-1N^{cage(K114AK116A)}
[SEQ. ID NO. 40]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILVANGADVNAYFGTTPLHLAAAHGRLEIV
EVLLKNGADVNAQDVWGITPLHLAAYNGHLEIVEVLLKYGADVNAHDTRGWTPLHLA
AINGHLEIVEVLLKNVADVNAQDRSGKTPFDLAIDNGNEDIAEVLQKAAKLNGSTSGSV
TTLSGLSGEQGPSGDMTTEEDSATHIKFSKRDEDGRELAGATMELRDSSGKTISTWISDG
HVKDFYLYPGKYCLVGSSEIITRNYGKTTIKEVVEIFDNDKNIQVLAFNTHTDNIEWAPIK
AAQLTRPNAELVELEIDTLHGVKTIRVTPDHPVYTKNRGYVRADELTDDDELVVAIENL
YGDEVNGYFQGMEAETYIGALASRAIVSNEDTYDIQTSTHNFFAND

TABLE 1F
Proteins with tuned cage lengths or no cage.

| Protein identifier | Domain structure |
|---|---|
| αHER2-SpyN-eNrdJ-1N^{cage(1-39)} | FLAG-αHER2-SpyN¹⁻⁷³-NrdJ-1N(C76V)^{cage(K104EK119A}^{_{—}}¹⁻³⁹⁾ |
| αHER2-SpyN-eNrdJ-1N^{cage(1-38)} | FLAG-αHER2-SpyN¹⁻⁷³-NrdJ-1N(C76V)^{cage(K104EK119A}^{_{—}}¹⁻³⁸⁾ |
| αHER2-SpyN-eNrdJ-1N^{cage(1-37)} | FLAG-αHER2-SpyN¹⁻⁷³-NrdJ-1N(C76V)^{cage(K104EK119A}^{_{—}}¹⁻³⁷⁾ |
| αHER2-SpyN-eNrdJ-1N^{cage(1-36)} | FLAG-αHER2-SpyN¹⁻⁷³-NrdJ-1N(C76V)^{cage(K104EK119A}^{_{—}}¹⁻³⁶⁾ |
| αHER2-SpyN-cNrdJ-1N^{cage(1-33)} | FLAG-αHER2-SpyN¹⁻⁷³-NrdJ-1N(C76V)^{cage(K104EK119A}^{_{—}}¹⁻³³⁾ |
| αHER2-SpyN-eNrdJ-1N^{cage(1-32)} | FLAG-αHER2-SpyN¹⁻⁷³-NrdJ-1N(C76V)^{cage(K104EK119A}^{_{—}}¹⁻³²⁾ |
| αHER2-SpyN-eNrdJ-1N^{cage(1-31)} | FLAG-αHER2-SpyN¹⁻⁷³-NrdJ-1N(C76V)^{cage(K104EK119A}^{_{—}}¹⁻³¹⁾ |
| αHER2-SpyN-eNrdJ-1N^{cage(1-30)} | FLAG-αHER2-SpyN¹⁻⁷³-NrdJ-1N(C76V)^{cage(K104EK119A}^{_{—}}¹⁻³⁰⁾ |
| αHER2-SpyN-eNrdJ-1N^{cage(1-29)} | FLAG-αHER2-SpyN¹⁻⁷³-NrdJ-1N(C76V)^{cage(K104EK119A}^{_{—}}¹⁻²⁹⁾ |
| αHER2-SpyN-eNrdJ-1N^{cage(1-26)} | FLAG-αHER2-SpyN¹⁻⁷³-NrdJ-1N(C76V)^{cage(K104EK119A}^{_{—}}¹⁻²⁶⁾ |
| αHER2-SpyN-eNrdJ-1N^{cage(1-20)} | FLAG-αHER2-SpyN¹⁻⁷³-NrdJ-1N(C76V)^{cage(K104EK119A}^{_{—}}¹⁻²⁰⁾ |
| αHER2-SpyN-NrdJ-1N | FLAG-αHER2-SpyN¹⁻⁷³-NrdJ-1N |
| NrdJ-1C-SpyC-αEGFR | NrdJ-1C-SpyC⁷⁴⁻¹¹³-αEGFR-Myc |

Protein primary structures from Table IF (here shown with N-terminal His₆-SUMO tag):

His₆-SUMO-αHER2-SpyN-eNrdJ-1N^{cage(1-39)}
[SEQ. ID NO. 41]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFFANDILVH
His₆-SUMO-αHER2-SpyN-eNrdJ-1N^{cage(1-38)}
[SEQ. ID NO. 42]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFFANDILV
His₆-SUMO-αHER2-SpyN-eNrdJ-1N^{cage(1-37)}
[SEQ. ID NO. 43]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFFANDIL
His₆-SUMO-αHER2-SpyN-eNrdJ-1N^{cage(1-36)}
[SEQ. ID NO. 44]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFFANDI
His₆-SUMO-αHER2-SpyN-eNrdJ-1N^{cage(1-33)}
[SEQ. ID NO. 45]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFFA
His₆-SUMO-αHER2-SpyN-eNrdJ-1N^{cage(1-32)}
[SEQ. ID NO. 46]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNFF
His₆-SUMO-αHER2-SpyN-eNrdJ-1N^{cage(1-31)}
[SEQ. ID NO. 47]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHNF
His₆-SUMO-αHER2-SpyN-eNrdJ-1N^{cage(1-30)}
[SEQ. ID NO. 48]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STHN
His₆-SUMO-αHER2-SpyN-eNrdJ-1N^{cage(1-29)}
[SEQ. ID NO. 49]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
STH
His₆-SUMO-αHER2-SpyN-eNrdJ-1N^{cage(1-26)}
[SEQ. ID NO. 50]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNEDTYDIQT
His₆-SUMO-αHER2-SpyN-eNrdJ-1N^{cage(1-20)}
[SEQ. ID NO. 51]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQGMEAETYIGKLKSRAIVSNED
His₆-SUMO-αHER2-SpyN-NrdJ-1N
[SEQ. ID NO. 52]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNAKDEYGLTPLYLATAHGHL
EIVEVLLKNGADVNAVDAIGFTPLHLAAFIGHLEIAEVLLKHGADVNAQDKFGKTAFDIS
IGNGNEDLAEILQKLNGSTSGSVTTLSGLSGEQGPSGDMTTEEDSATHIKFSKRDEDGRE
LAGATMELRDSSGKTISTWISDGHVKDFYLYPGKYCLVGSSEIITRNYGKTTIKEVVEIFD
NDKNIQVLAFNTHTDNIEWAPIKAAQLTRPNAELVELEIDTLHGVKTIRVTPDHPVYTKN
RGYVRADELTDDDELVVAIENLYGDEVNGYFQG
His₆-SUMO-NrdJ-1C-SpyC-αEGFR
[SEQ. ID NO. 53]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
MEAKTYIGKLKSRKIVSNEDTYDIQTSTHNFFANDILVHNSFVETAAPDGYEVATPIEFTV
NEDGQVTVDGEATEGDAHTGSTSGSDLGKKLLEAARAGQDDEVRILMANGADVNADD
TWGWTPLHLAAYQGHLEIVEVLLKNGADVNAYDYIGWTPLHLAADGHLEIVEVLLKN
GADVNASDYIGDTPLHLAAHNGHLEIVEVLLKHGADVNAQDKFGKTAFDISIDNGNEDL
AEILQKLNEQKLISEEDL

TABLE 1G
Protein used as decoy in NOT gates.

| Protein identifier | Domain structure |
|---|---|
| Decoy-αEGFR | FLAG-αEGFR-SpyN^{1-74(K31E)}-NrdJ- |
| | 1N(C76V)^{cage(K104EK114AK116AK119A}^{_{—}}¹⁻²⁹⁾ |

Protein primary structure from Table 1G (here shown with N-terminal His₆-SUMO tag):

His₆-SUMO-Decoy-αEGFR
[SEQ. ID NO. 54]
MGSSHHHHHHGSGLVPRGSASMSDSEVNQEAKPEVKPEVKPETHINLKVSDGSSEIFFKI
KKTTPLRRLMEAFAKRQGKEMDSLRFLYDGIRIQADQTPEDLDMEDNDIIEAHREQIGG
DYKDDDDKDLGKKLLEAARAGQDDEVRILMANGADVNADDTWGWTPLHLAAYQGH
LEIVEVLLKNGADVNAYDYIGWTPLHLAADGHLEIVEVLLKNGADVNASDYIGDTPLHL
AAHNGHLEIVEVLLKHGADVNAQDKFGKTAFDISIDNGNEDLAEILQKLNGSTSGSVTT
LSGLSGEQGPSGDMTTEEDSATHIKFSERDEDGRELAGATMELRDSSGKTISTWISDGHV
KDFYLYPGKYCLVGSSEIITRNYGKTTIKEVVEIFDNDKNIQVLAFNTHTDNIEWAPIKAA
QLTRPNAELVELEIDTLHGVKTIRVTPDHPVYTKNRGYVRADELTDDDELVVAIENLYG
DEVNGYFQGMEAETYIGALASRAIVSNEDTYDIQTSTH

TABLE 1H
Caged split intein fragments.

Protein identifier
eNrdJ-1N^{cage}
eNrdJ-1C^{cage}
NrdJ-1N^{cage}
NrdJ-1C^{cage}
NpuN^{cage}
NpuC^{cage}
Gp41-1N^{cage}
Gp41-1C^{cage}
Gp41-8N^{cage}
Gp41-8C^{cage}

eNrdJ-1N^{cage}
CLVGSSEIITRNYGKTTIKEVVEIFDNDKNIQVLAFNTHTDNIEWAPIKAAQLTRPNAELV
ELEIDTLHGVKTIRVTPDHPVYTKNRGYVRADELTDDDELVVAIENLYGDEVNGYFQG
MEAETYIGKLKSRAIVSNEDTYDIQTSTHNFFAND
eNrdJ-1C^{cage}
NAELVELEIKTLHGVKTIRVTPDHPVYTKNRGYVRADELTDDDELVVAIENLYGDEVNG
YFQGMEAKTYIGKLKSRKIVSNEDTYDIQTSTHNFFANDILVHN
NrdJ-1N^{cage}
CLVGSSEIITRNYGKTTIKEVVEIFDNDKNIQVLAFNTHTDNIEWAPIKAAQLTRPNAELV
ELEIDTLHGVKTIRCTPDHPVYTKNRGYVRADELTDDDELVVAIENLYGDEVDGYFQG
MEAKTYIGKLKSRKIVSNEDTYDIQTSTHNFFAND
NrdJ-1C^{cage}
NAELVELEIDTLHGVKTIRCTPDHPVYTKNRGYVRADELTDDDELVVAIENLYGDEVDG
YFQGMEAKTYIGKLKSRKIVSNEDTYDIQTSTHNFFANDILVHN
NpuN^{cage}
CLSYETKILTVEYGLLPIGKIVEKRIECTVYSVDNNGNIYTQPVAQWHDRGKQKVFEYCL
KDGSLIRATKDHKFMTVDGQMLPIKEIFRRKLDLMRVDNLPNENLYGDEVDGYFQGIEI
ATEKYLGEQNVYDIGVERDHNFALENGGYFQGIEIATEKYLGEQNVYDIGVERDHNFAL
ENG
NpuC^{cage}
GEQEVFEYCLEDGSLIRATKDHKFMTVDGQMLPIDEIFERELDLMRVDNLPNENLYGDE
VDGYFQGIKIATRKYLGKQNVYDIGVERDHNFALKNGFIASN
Gp41-1N^{cage}
CLDLKTQVQTPQGMKEISNIQVGDLVLSNTGYNEVLNVFPKSKKKSYKITLEDGKEIICS
EEHLFPTQTGEMNISGGLKEGMCLYVKEGNLYGDEVDGYFQGMMLKKILKIEELDERE
LIDIEVSGNHLFYAND
Gp41-1C^{cage}
SKKKSYKITLEDGKEIICSEEHLFPTQTGEMNISGGLKEGMCLYVKEGNLYGDEVDGYF
QGMMLKKILKIEELDERELIDIEVSGNHLFYANDILTHN
Gp41-8N^{cage}
CLSLDTMVVTNGKAIEIRDVKVGDWLESECGPVQVTEVLPIIKQPVFEIVLKSGKKIRVS
ANHKFPTKDGLKTINSGLKVGDFLRSRAKENLYGDEVDGYFQGMCEIFENEIDWDEIASI
EYVGVEETIDINVTNDRLFFANG
Gp41-8C^{cage}
IIKQPVFEIVLKSGKKIRVSANHKFPTKDGLKTINSGLKVGDFLRSRAKENLYGDEVDGY
FQGMCEIFENEIDWDEIASIEYVGVEETIDINVTNDRLFFANGILTHN

### Protein Sizes

The following Table 2 presents the calculated molar mass (Mr (Cal.)) in g/mol and determined molar mass (Mr (Det.)) in g/mol for a protein whose identifier (Protein ID) and domain structure is provided. The molar masses were determined by mass spectrometry (see Methods and Materials section).

TABLE 2
Calculated molar mass (Mr (Cal.)) in g/mol and determined molar mass (Mr (Det.)) in g/mol for proteins.

| Protein ID | Domain structure | Mr (Cal.) | Mr (Det.) |
|---|---|---|---|
| SpyCatcher003 | FLAG-αHER2-SpyCatcher003-αEGFR-Myc | 45,316.0 | 45,316.0 |
| SpyTag003-Cys | His₆-SpyTag003-SUMO-Cys | 15,591.0 | 15,591.6 |
| SpyTag003-AF594 | His₆-SpyTag003-SUMO-Cys Alexa Fluor 594 conjugate | 16,478.5 | 16,478.5 |
| SpyTag003-biotin | His₆-SpyTag003-SUMO-Cys biotin conjugate | 16,043.0 | 16,043.0 |
| SpyTag003-DNP | His₆-SpyTag003-SUMO-Cys dinitrophenol conjugate | 16,087.7 | 16,087.0 |
| SpyTag003^{D117A}-Cys | His₆-SpyTag003^{D117A}-SUMO-Cys | 15,547.5 | 15,547.1 |
| SpyTag003^{D117A}-AF594 | His₆-SpyTag003^{D117A}-SUMO-Cys Alexa Fluor 594 conjugate | 16,435.0 | 16,434.8 |
| SpyTag003-APEX2 | His₆-SpyTag003-HA-APEX2 | 32,493.0 | 32,492.0 |
| αHER2-Cys DARPin | αHER2-HA-Cys | 14,700.5 | 14,699.5 |
| αHER2-AF594 DARPin | αHER2-HA-Cys Alexa Fluor 594 conjugate | 15,587.5 | 15,586.0 |
| αEGFR-Cys DARPin | αEGFR-HA-Cys | 18,397.5 | 18,396.2 |
| αEGFR-AF594 DARPin | αEGFR-HA-Cys Alexa Fluor 594 conjugate | 19,284.5 | 19,284.0 |
| αEpCAM-Cys DARPin | αEpCAM-HA-Cys | 18,452.7 | 18,451.5 |
| αEpCAM-AF594 DARPin | αEpCAM-HA-Cys Alexa Fluor 594 conjugate | 19,339.7 | 19,339.0 |
| SpyN¹⁻²⁴-NpuN^{cage}-FKBP | FLAG-αHER2-SpyN¹⁻²⁴-NpuN^{cage}-FKBP | 50,637.8 | 50,637.0 |
| SpyN¹⁻⁴²-NpuN^{cage}-FKBP | FLAG-αHER2-SpyN¹⁻⁴²-NpuN^{cage}-FKBP | 52,650.1 | 52,648.3 |
| SpyN¹⁻⁵⁵-NpuN^{cage}-FKBP | FLAG-αHER2-SpyN¹⁻⁵⁵-NpuN^{cage}-FKBP | 54,056.6 | 54,055.2 |
| SpyN¹⁻⁷³-NpuN^{cage}-FKBP | FLAG-αHER2-SpyN¹⁻⁷³-NpuN^{cage}-FKBP | 56,228.1 | 56,227.8 |
| SpyN¹⁻⁸²-NpuN^{cage}-FKBP | FLAG-αHER2-SpyN¹⁻⁸²-NpuN^{cage}-FKBP | 57,160.1 | 57,158.6 |
| SpyN¹⁻⁹⁰-NpuN^{cage}-FKBP | FLAG-αHER2-SpyN¹⁻⁹⁰-NpuN^{cage}-FKBP | 57,991.0 | 57,989.9 |
| FRB-NpuC^{cage}-SpyC²⁵⁻¹¹³ | FRB-NpuC^{cage}-SpyC²⁵⁻¹¹³-αEGFR-Myc | 52,518.6 | 52,517.0 |
| FRB-NpuC^{cage}-SpyC⁴³⁻¹¹³ | FRB-NpuC^{cage}-SpyC⁴³⁻¹¹³-αEGFR-Myc | 50,506.4 | 50,504.1 |
| FRB-NpuC^{cage}-SpyC⁵⁶⁻¹¹³ | FRB-NpuC^{cage}-SpyC⁵⁶⁻¹¹³-αEGFR-Myc | 49,099.8 | 49,101.1 |
| FRB-NpuC^{cage}-SpyC⁷⁴⁻¹¹³ | FRB-NpuC^{cage}-SpyC⁷⁴⁻¹¹³-αEGFR-Myc | 46,928.4 | 46,927.1 |
| FRB-NpuC^{cage}-SpyC⁸³⁻¹¹³ | FRB-NpuC^{cage}-SpyC⁸³⁻¹¹³-αEGFR-Myc | 46,040.4 | 46,038.6 |
| FRB-NpuC^{cage}-SpyC⁹¹⁻¹¹³ | FRB-NpuC^{cage}-SpyC⁹¹⁻¹¹³-αEGFR-Myc | 45,137.4 | 45,135.0 |
| SpyN¹⁻⁷³-NrdJ-1N^{cage}-FKBP | FLAG-αHER2-SpyN¹⁻⁷³-NrdJ-1N^{cage}FKBP | 52,197.4 | 52,196.6 |
| SpyN¹⁻⁷³-NrdJ-1N(C1A)^{cage}-FKBP | FLAG-αHER2-SpyN¹⁻⁷³-NrdJ-1N(C1A)^{cage}-FKBP | 52,161.3 | 52,159.4 |
| SpyN¹⁻⁷³-NrdJ-1N(C76V)^{cage}-FKBP | FLAG-αHER2-SpyN¹⁻⁷³-NrdJ-1N(C76V)^{cage}-FKBP | 52,193.3 | 52,191.0 |
| FRB-NrdJ-1C^{cage}-SpyC⁷⁴⁻¹¹³ | FRB-NrdJ-1C^{cage}-SpyC⁷⁴⁻¹¹³-αEGFR-Myc | 46,364.5 | 46,364.6 |
| FRB-NrdJ-1C^{cage(C76V)}-SpyC⁷⁴⁻¹¹³ | FRB-NrdJ-1C^{cage(C76V)}-SpyC⁷⁴⁻¹¹³-αEGFR-Myc | 46,360.5 | 46,359.0 |
| SpyN-NrdJ-1N^{cage(C76V)}-αHER2 | FLAG-αHER2-SpyN¹⁻⁷³-NrdJ-1N(C76V)^{cage} | 40,062.5 | 40,062.0 |
| αHER2-SpyN-eNrdJ-1N^{cage} | FLAG-αHER2-SpyN¹⁻⁷³-NrdJ-1N(C76V)^{cage(K104EK119A)} | 52,137.2 | 52,133.6 |
| αHER2-SpyN-eNrdJ-1N^{cage} Cys1-alkylated | FLAG-αHER2-SpyN¹⁻⁷³-NrdJ-1N(C76V)^{cage(K104EK119A)} Cys1-alkylated | 40,063.0 | 40,062.0 |
| αEGFR-SpyN-eNrdJ-1N^{cage} | FLAG-αEGFR-SpyN¹⁻⁷³-NrdJ-1N(C76V)^{cage(K104EK119A)} | 43,702.3 | 43,701.5 |
| αEpCAM-SpyN-eNrdJ-1N^{cage} | FLAG-αEpCAM-SpyN¹⁻⁷³-NrdJ-1N(C76V)^{cage(K104EK119A)} | 43,757.6 | 43,756.4 |
| SpyC-NrdJ-1C^{cage(C76V)}-αEGFR | NrdJ-1C^{cage(C76V)}-SpyC⁷⁴⁻¹¹³-αEGFR-Myc | 34,437.9 | 34,437.0 |
| αHER2-eNrdJ-1C^{cage}-SpyC | NrdJ-1C^{cage(D66KC76V)}-SpyC⁷⁴⁻¹¹³-αHER2-Myc | 30,753.1 | 30,752.0 |
| αEGFR-eNrdJ-1C^{cage}-SpyC | NrdJ-1C^{cage(D66KC76V)}-SpyC⁷⁴⁻¹¹³-αEGFR-Myc | 34,450.0 | 34,449.8 |
| αEpCAM-eNrdJ-1C^{cage}-SpyC | NrdJ-1C^{cage(D66KC76V)}-SpyC⁷⁴⁻¹¹³-αEpCAM-Myc | 34,505.3 | 34,505.2 |
| αHER2-SpyN-eNrdJ-1N^{cage(K114A)} | FLAG-αHER2-SpyN¹⁻⁴²-NrdJ-1N(C76V)^{cage(K104EK114AK119A)} | 39,948.3 | 39,947.3 |
| αHER2-SpyN-eNrdJ-1N^{cage(K116A)} | FLAG-αHER2-SpyN¹⁻⁴²-NrdJ-1N(C76V)^{cage(K104EK116AK119A)} | 39,948.3 | 39,946.8 |
| αHER2-SpyN-eNrdJ-1N^{cage(R118A)} | FLAG-αHER2-SpyN¹⁻⁴²-NrdJ-1N(C76V)^{cage(K104ER118AK119A)} | 39,920.3 | 39,921.2 |
| αHER2-SpyN-eNrdJ-1N^{cage(K114AK116A)} | FLAG-αHER2-SpyN¹⁻⁴²-NrdJ-1N(C76V)^{cage(K104EK114AK116AK119A)} | 39,891.1 | 39,892.2 |
| αHER2-SpyN-eNrdJ-1N^{cage(K114AK116AR118A)} | FLAG-αHER2-SpyN¹⁻⁴²-NrdJ-1N(C76V)^{cage(K104EK114AK116AR118AK119A)} | 39,806.1 | 39,807.1 |
| αHER2-SpyN-eNrdJ-1N^{cage(A119K)} | FLAG-αHER2-SpyN¹⁻⁴²-NrdJ-1N(C76V)^{cage(K104E)} | 40,062.4 | 40063.0 |
| αEGFR-SpyN-eNrdJ-1N^{cage(K114AK116A)} | FLAG-αEGFR-SpyN¹⁻⁴²-NrdJ-1N(C76V)^{cage(K104EK114AK116AK119A)} | 43,643.4 | 43,641.8 |
| αEpCAM-SpyN-eNrdJ-1N^{cage(K114AK116A)} | FLAG-αEpCAM-SpyN¹⁻⁴²-NrdJ-1N(C76V)^{cage(K104EK114AK116AK119A)} | 43,588.1 | 43,587.1 |
| αHER2-SpyN-eNrdJ-1N^{cage(1-39)} | FLAG-αHER2-SpyN¹⁻⁷³-NrdJ-1N(C76V)^{cage(K104EK119A}^{_{—}}¹⁻³⁹⁾ | 40,468.0 | 40,467.0 |
| αHER2-SpyN-eNrdJ-1N^{cage(1-38)} | FLAG-αHER2-SpyN¹⁻⁷³-NrdJ-1N(C76V)^{cage(K104EK119A}^{_{—}}¹⁻³⁸⁾ | 40,330.8 | 40,331.4 |
| αHER2-SpyN-eNrdJ-1N^{cage(1-37)} | FLAG-αHER2-SpyN¹⁻⁷³-NrdJ-1N(C76V)^{cage(K104EK119A}^{_{—}}¹⁻³⁷⁾ | 40,231.7 | 40,231.2 |
| αHER2-SpyN-eNrdJ-1N^{cage(1-36)} | FLAG-αHER2-SpyN¹⁻⁷³-NrdJ-1N(C76V)^{cage(K104EK119A}^{_{—}}¹⁻³⁶⁾ | 40,118.5 | 40,118.3 |
| αHER2-SpyN-eNrdJ-1N^{cage(1-33)} | FLAG-αHER2-SpyN¹⁻⁷³-NrdJ-1N(C76V)^{cage(K104EK119A}^{_{—}}¹⁻³³⁾ | 39,776.2 | 39,775.3 |
| αHER2-SpyN-eNrdJ-1N^{cage(1-32)} | FLAG-αHER2-SpyN¹⁻⁷³-NrdJ-1N(C76V)^{cage(K104EK119A}^{_{—}}¹⁻³²⁾ | 39,705.1 | 39,704.7 |
| αHER2-SpyN-eNrdJ-1N^{cage(1-31)} | FLAG-αHER2-SpyN¹⁻⁷³-NrdJ-1N(C76V)^{cage(K104EK119A}^{_{—}}¹⁻³¹⁾ | 39,557.9 | 39,556.8 |
| αHER2-SpyN-eNrdJ-1N^{cage(1-30)} | FLAG-αHER2-SpyN¹⁻⁷³-NrdJ-1N(C76V)^{cage(K104EK119A}^{_{—}}¹⁻³⁰⁾ | 39,410.7 | 39,410.2 |
| αHER2-SpyN-eNrdJ-1N^{cage(1-29)} | FLAG-αHER2-SpyN¹⁻⁷³-NrdJ-1N(C76V)^{cage(K104EK119A}^{_{—}}¹⁻²⁹⁾ | 39,296.6 | 39,295.6 |
| αHER2-SpyN-eNrdJ-1N^{cage(1-26)} | FLAG-αHER2-SpyN¹⁻⁷³-NrdJ-1N(C76V)^{cage(K104EK119A}^{_{—}}¹⁻²⁶⁾ | 38,971.3 | 38,970.1 |
| αHER2-SpyN-eNrdJ-1N^{cage(1-20)} | FLAG-αHER2-SpyN¹⁻⁷³-NrdJ-1N(C76V)^{cage(K104EK119A}^{_{—}}¹⁻²⁰⁾ | 38,249.5 | 38,248.6 |
| αHER2-SpyN-NrdJ-1N | FLAG-αHER2-SpyN¹⁻⁷³-NrdJ-1N | 34,426.4 | 34,425.2 |
| NrdJ-1C-SpyC-αEGFR | NrdJ-1C-SpyC⁷⁴⁻¹¹³-αEGFR-Myc | 27,334.1 | 27,333.7 |
| Decoy-αEGFR | FLAG-αEGFR-SpyN^{1-74(K31E)}-NrdJ-1N(C76V)^{cage(K104EK114AK116AK119A)}^{_{—}}¹⁻²⁹ | 42,880.3 | 42,881.0 |

### Methods and Materials

### General Materials

Common reagents and chemicals were purchased from MilliporeSigma (Burlington, MA) unless stated otherwise. 2,4-dinitrochlorobenzene (CAS NO 97-00-7), tert-butyl bromoacetate (CAS NO. 5292-43-3), N-hydroxysuccinimide (CAS NO. 6066-82-6), N,N′-Dicyclohexylcarbodiimide (CAS NO. 538-75-0), Celite 545 (CAS NO. 68855-54-9), and 1-(2-aminoethyl)maleimide hydrochloride (CAS NO. 134272-64-3) were purchased from MilliporeSigma (Burlington, MA). 2-[2-(2-aminoethoxy) ethoxy]ethanol (CAS NO 86770-74-3), and Biotin-PEG3-amine (CAS NO 359860-27-8) were purchased from Ambeed (Arlington Heights, IL). Triethylamine (CAS NO 121-44-8) was purchased from Thermo Fischer Scientific (Waltham, MA). Dithiothreitol (DTT), isopropyl-β-thiogalactopyranoside (IPTG) and bovine serum albumin (BSA) were obtained from Gold Biotechnology (Burlington, MA). Alexa Fluor 594 C5 maleimide was purchased from BroadPharm (San Diego, CA). Biotin maleimide (CAS NO 116919-18-7) was purchased from MilliporeSigma (Burlington, MA).

Oligonucleotide primers were purchased from MilliporeSigma (Burlington, MA). gBlock® Gene Fragments were purchased from Integrated DNA Technologies. PrimeSTAR HS DNA Polymerase was purchased from Takara Bio (Kusatsu, Japan). Gibson Assembly Master Mix was purchased from New England Biolabs (Ipswich, MA). DNA purification kits were purchased from Qiagen (Valencia, CA). PCR Purification&Gel extract columns were purchased from Thomas Scientific (Swedesboro, NJ). All plasmid sequencing was performed by GENEWIZ (South Plainfield, NJ) or alternatively by Plasmidosaurus (Eugene, OR). Hoechst 33342 solution was obtained from Invitrogen (Carlsbad, CA).

Nickel nitrilotriacetic acid (Ni-NTA) resin was obtained from Thermo Fisher Scientific (Waltham, MA). MOPS-SDS running buffer was obtained from Boston Bioproducts (Ashland, MA). Criterion Cassettes, acrylamide, ammonium persulfate, Tetramethylethylenediamine, and Econo-Pac® 10DG Columns were obtained from Bio-Rad (Hercules, CA). Nitrocellulose membrane (0.45 μm) for western blotting was purchased from Thermo Fisher Scientific (Waltham, MA).

NeutrAvidin™ Rhodamine Red™-X conjugate was purchased from Thermo Fischer Scientific (Waltham. MA). Streptavidin-Saporin (Streptavidin-ZAP) was purchased from Advanced Targeting Systems (Carlsbad, CA).

Dulbecco's Modified Eagle Medium (Gibco), RPMI-1640 Medium (Gibco), McCoy's 5a Medium Modified (Gibco), F-12K Medium (Gibco), Dulbecco's phosphate-buffered saline (DPBS, Gibco), Penicillin-Streptomycin (5,000 U/mL), Trypsin-EDTA (0.25%), Trypsin-EDTA (0.05%), and Falcon™ Standard Tissue Culture Dishes were purchased from Thermo Fisher Scientific (Waltham. MA). Mammary Epithelial Cell Growth Medium kit was purchased from MilliporeSigma (Burlington, MA). Fetal Bovine Serum (Heat Inactivated) was purchased from Bio-Techne (Minneapolis, MN). XTT Cell Viability Kit was purchased from Cell Signal Technologies (Danvers, MA). Glass bottom 24 well plates were purchased from Cellvis (Mountain View. CA).

Human cell lines K562^{EpCAMlo} (wildtype, CCL-243), K562^{HER2+}, K562^{EGFR+}, K562^{HER2+/EGFR+}, K562^{HER2+/EpCAMhi}, K562^{HER2+/EGFR+/EpCAMhi} were generous gifts from Prof. David Baker (University of Washington, WA). Human cell lines MCF-10a (CRL-10317), MCF-7 (HTB-22), LoVo (CCL-229), A594 (CCL-185) were generous gifts from Prof. Yibin Kang (Princeton University, NJ). Human cell line Sk-br-3 (HTB-30) was a generous gift from Dr. Stan Lipkowitz (National Cancer Institute, Bethesda, MD). Human cell line OE19 (JROECL19) was purchased from MilliporeSigma (Burlington, MA). Human cell lines HCT-116 (CCL-247) and A431 (CRL-1555) were purchased from ATCC (Manassas, VA)

### DNA Cloning

Expression vectors: All expression vectors were based on a pET backbone vector with a gene for kanamycin resistance. DNA fragments amplified by Polymerase Chain Reaction (PCR) were inserted into a gene cassette using Gibson Assembly. The gene cassette included an upstream T7 promoter under regulation of a lac operator, an ATG start codon in frame with a N-terminal His₆-SUMO fusion tag when specified and ended with a TAA stop codon and a T7-terminator sequence.
Polymerase Chain Reaction: Site-directed mutagenesis (including missense substitutions, insertions, and deletions), in addition to amplifications of gene inserts and plasmid backbones were performed by PCR. To a solution of 10 μL 1 ng/μL plasmid, 1 μM forward primer, 1 μM reverse primer was added 10 μL 2x PrimeSTAR DNA polymerase Master Mix. The mixture was used in a PCR reaction. The completed PCR reaction was restriction digested with 20 units of DpnI for 1 hr at 37° C. before the PCR amplicon was isolated by a general PCR clean up protocol. The purified PCR amplicon was then used in a Gibson assembly reaction (see below). Alternatively, full plasmid amplicons from site directed mutagenesis was used directly in heat-shock transformations of chemically competent Escherichia coli DH5α, which were plated on LB-agar plates supplemented with 50 μg/mL kanamycin. Single colonies were picked, grown and plasmid isolated before being verified by Sanger sequencing.
Gibson assembly: DNA components prepared by PCR using primers designed to have 15-20 base pair overlaps were used in Gibson Assembly reactions. Gibson Assembly reactions were set up by mixing 2 μL DNA fragments (100 ng plasmid backbone amplicon, 3-5 molar excess gene insert amplicon) with 2 μL 2× NEBuilder® HiFi DNA Assembly master mix. The reaction was incubated for 15 min at 50° C., cooled and thereafter used in a heat-shock transformation of chemically competent Escherichia coli DH5α, which were plated on LB-agar plates supplemented with 50 μg/mL kanamycin. Single colonies were picked, grown and plasmid isolated before being verified by Sanger sequencing.

### Recombinant Protein Expression and Purification

Overnight starter culture: Chemically competent Escherichia coli BL21(DE3) were heat-shock transformed with pET vector carrying gene cassettes for the protein of interest and a kanamycin resistance gene. Cells were grown overnight at 37° C. in 8 mL LB medium supplemented with 50 μg/mL kanamycin.
Recombinant protein expression: An overnight culture was used to inoculate an expression culture of 1 L LB medium supplemented with 50 μg/mL kanamycin. In general, the expression culture was incubated at 37° C., 200 RPM until it reached an optical density at 600 nm of 0.4 units, whereafter it was cooled for 20 min at 18° C., 200 RPM. The expression was then induced by the addition of 0.1 mL 1 M IPTG and the culture left overnight at 18° C. 200 RPM. For expression of full-length NrdJ-1 used in the crystallography study and for any SpyTag003 and stand-alone DARPin constructs the expression culture was incubated at 37° C., 200 RPM until it reached an optical density at 600 nm of 0.6 units. The expression was then induced by the addition of 0.1 mL 1 M IPTG and the culture left for 4 hr at 37° C., 200 RPM. For any condition, cells were harvested by centrifugation at 3,500 g, 18° C. for 20 min and suspended in lysis buffer 20 mL 50 mM NaH₂PO₄ (pH 8.0), 300 mM NaCl, 20 mM imidazole, supplemented with 1 mM dithiothreitol (DTT) and 1 mM phenylmethylsulfonyl fluoride (PMSF). Cell suspensions were then either stored at −20° C. until further use or used directly.
Recombinant protein extraction and affinity purification: All following procedures were performed at 4° C. Total soluble protein was extracted by subjecting the cell suspension to sonication for 3 min 20 s using a duty cycle of 20 s on, 30 s off, at 30% amplitude while cooled on an ice bath, after which a cleared lysate was produced by centrifugation at 35.000 g, 4° C. for 20 min. Ni²⁺-nitrilotriacetic acid (NTA) column was prepacked from 2 mL resin slurry per liter culture and preequilibrated with lysis buffer. The cleared lysate was passed through the column and the flow-through discarded. The column was then washed with 50 mL lysis buffer, before the protein was eluted using 6 mL lysis buffer supplemented with 250 mM imidazole. His₆-SUMO tagged proteins were treated overnight with His₆-Ulp1 protease while dialyzed against lysis buffer supplemented with 1 mM DTT. A new Ni²⁺-nitrilotriacetic acid (NTA) column was prepacked from 3 mL resin slurry per liter culture and preequilibrated with lysis buffer. The dialyzed sample was then passed through the column to remove any cleaved His₆-SUMO tag and His₆-Ulp1 protease. The flow-through and an additional 6 mL lysis buffer passed through the column was collected, combined, and concentrated to 0.5 mL. Proteins that were not His₆-SUMO tagged did not go through the process of Ulp1 protease treatment, dialysis, and the second affinity step but was concentrated directly.
Size-exclusion chromatography and recombinant protein product analysis: The concentrated sample was filtered through a 0.22 μm spin filter and the flow-through applied to size-exclusion chromatography passing it through a Superdex 200 10/300 GL (Cytiva Life Sciences) column with 100 mM NaH₂PO₄ (pH 7.2), 150 mM NaCl, 1 mM ethylenediaminetetraacetic acid (EDTA), and 1 mM DTT as eluent at a flowrate of 0.5 mL/min performed on a AKTA Fast Performance Liquid Chromatography (GE Healthcare) system. Pure fractions identified by SDS-PAGE, validated by analytical RP-HPLC, and the protein mass confirmed by Electrospray Ionization Time-of-Flight Mass Spectrometry (ESI-TOF MS). Pure fractions were supplemented with 10% v/v glycerol, aliquoted and flash-frozen in liquid nitrogen before being stored at −80° C. until further use.

### SDS-PAGE

Samples were boiled in Laemmli buffer and run on a 10% BIS-TRIS SDS-PAGE gel in MOPS-SDS running buffer. For protein staining the gel was incubated with 2.5 mg/L Coomassie Brilliant Blue R-250 dissolved in 10% v/v acetic acid, 40% v/v methanol, 50% v/v water, later to be destained in the solution without dye before being imagined.

### Analytical RP-HPLC

Analytical Reversed-Phase High-Pressure Liquid Chromatography (RP-HPLC) was performed on a Vydac 218 ms C18 column (5 μM particle size, 4.6×150 mm dimension) at a flow rate of 1 ml/min. The mobile phase comprised of 0.1% v/v trifluoroacetic acid in water (solvent A) and 90% acetonitrile, 0.1% TFA in water (solvent B).

### Mass Spectrometry

Electrospray Ionization Time-of-Flight Mass Spectrometry (ESI-TOF MS) was performed on a Bruker Daltonics micrOTOF-Q II mass spectrometer. Masses quoted are for the monoisotopic mass as the singly protonated species.

### Western Blotting

Samples were run on an SDS-PAGE gel. For western blotting, the gel was used for a transfer reaction onto a nitrocellulose membrane, which was subsequently blocked with TBS-T (25 mM Tris, 150 mM NaCl, 0.1% v/v Tween-20, pH 7.7) supplemented with 4% w/v skimmed milk powder for 1 hr at RT. The membrane was washed 3×5 min with TBS-T and incubated with primary antibodies at specified dilution (Table 4) on an orbital shaker for 1 hr at room temperature or alternatively overnight at 4° C. After washing 3×5 min with TBS-T, the IRDye secondary antibody or alternatively IRDye Streptavidin were applied for 30 min to 1 hour at room temperature, before imaging on a Li-Cor Odyssey imager (Li-Cor).

### X-Ray Crystallography

Recombinant expression and purification: Full-length NrdJ-1 with CIA and N145A was expressed and purified as a His6-SUMO tagged protein according to the protocol above. SGG and SEI were used as N- and C-terminus of extein sequences. The protein was dialyzed against a buffer of 25 mM HEPES (pH 7.5), 150 mM NaCl, then concentrated to 40 mg/mL, and finally flash-frozen as aliquots in liquid nitrogen before being stored at −80 dC for further use. Protein crystallization: Initial crystallization conditions were established using the SaltRx HT screen (Hampton Research), where an aliquot of 0.3 μL protein solution was mixed with 0.3 μL screening solution per condition using a Phoenix crystallization robot (Art Robbins). Crystals were grown at 4° C. by the sitting-drop vapor diffusion method. Best results were obtained with sodium formate conditions. A focused screen was centered on conditions with increasing concentrations of sodium formate and at various pH values. Optimal crystals were obtained after one week using a solution of 4.5 M sodium formate (pH 7.0).
Diffraction data collection and processing: Diffraction data was obtained at the National Synchrotron Light Source II (Brookhaven National Laboratory), beamline 17-ID-1. The data was processed using the XDS package²⁷. The phase information was determined by molecular replacement using PHASER in the CCP4 suit²⁸ and an in silico AlphaFold2 model^{30, 31} of full-length NrdJ-1 as input. Iterative rounds of model building in Coot³¹ and refinements in PHENIX Refine (version 1.17_3644)³² were done to obtain the final structure. Data collection and refinement statistics are displayed in Table 3.

### Protein Conjugation Reactions

Protein cysteine reduction: Purified protein with a C-terminal cysteine (SpyTag003-Cys, SpyTag003^{D117A}-Cys, αHER2-Cys DARPin, αEGFR-Cys DARPin, αEpCAM-Cys DARPin, or SpyN-eNrdJ-1N^{cage}-αHER2) was reduced with 1 mM DTT for 20 min on ice to prepare it for conjugation chemistry. The sample was then washed with buffer 100 mM NaH₂PO₄ (pH 7.2), 150 mM NaCl₂, 1 mM EDTA using spin-filtration to remove any remaining small molecule thiols. The reduced protein was then either flash-frozen in liquid nitrogen and stored at −80° C. for later use or used directly in conjugation reactions as described in the following paragraphs. Iodoacetamide conjugates: The protein was mixed with freshly prepared iodoacetamide at 2 molar excess and incubated for 20 min at room temperature in the dark. The reaction was monitored by RP-HPLC and ESI-TOF MS and quenched with 1 mM DTT once completed. The reaction was then passed over an Econo-Pac® 10DG Column using 100 mM NaH₂PO₄ (pH 7.2), 150 mM NaCl, 1 mM EDTA, 10% v/v glycerol as eluent. The product was validated by RP-HPLC and ESI-TOF MS, flash-frozen and stored at −80° C.
Alexa Fluor 594 conjugates: The protein was mixed with Alexa Fluor 594 maleimide at 1.2 molar excess and incubated at room temperature in the dark under gentle agitation. The reaction was monitored by RP-HPLC and ESI-TOF MS and quenched with 1 mM DTT once completed. The reaction was then passed over an Econo-Pac® 10DG Column using 100 mM NaH₂PO₄ (pH 7.2), 150 mM NaCl, 1 mM EDTA, 10% v/v glycerol as eluent. The product was validated by RP-HPLC and ESI-TOF MS, flash-frozen and stored at −80° C.
Biotin conjugates: The protein was mixed with biotin-maleimide at 1.2 molar excess of biotin maleimide and incubated at room temperature under gentle agitation. The reaction was monitored by RP-HPLC and ESI-TOF MS and quenched with 1 mM DTT once completed. The reaction was then washed with 100 mM NaH₂PO₄ (pH 7.2), 150 mM NaCl, 1 mM EDTA by spin-filtration and concentrated. The product was validated by RP-HPLC and ESI-TOF MS, flash-frozen and stored at −80° C.
Dinitrophenol conjugates: The protein was mixed with 2,4-dinitrophenyl-maleimide at 1.2 molar excess and incubated at room temperature under gentle agitation. The reaction was monitored by RP-HPLC and ESI-TOF MS and quenched with 1 mM DTT once completed. The reaction was then washed with 100 mM NaH₂PO₄ (pH 7.2), 150 mM NaCl, 1 mM EDTA by spin-filtration and concentrated. The product was validated by RP-HPLC and ESI-TOF MS, flash-frozen and stored at −80° C.

### Screening for Optimal SpyCatcher003 Split Site

In a standard test-tube containing 24 μL splicing buffer of 100 mM NaH₂PO₄ (pH 7.2), 150 mM NaCl, 1 mM EDTA, 1 mM DTT, 10% v/v glycerol was mixed 8 μL, 5 μM of each of the split-SpyCatcher003 actuator components and 8 μL, 10 μM SpyTag003 or conjugates thereof. Splicing was initiated either by addition of 8 μL, 50 UM rapamycin (DMSO) or 1 μL, 40×TEV protease. Buffered solution was added to compensate for volume differences when reaction components were omitted in control reactions. The reaction was left for 24 hr at 37° C. before further analysis by SDS-PAGE followed by western blotting.

### Mammalian Cell Culturing

Mammalian cell lines were cultured in media as detailed in Table 5 supplemented with 100 U/mL penicillin/streptomycin in an incubator at 37° C. and 5% CO₂. All cell lines were regularly tested free of mycoplasma.

### Confocal Microscopy

The cell sample was incubated for 30 min at room temperature with Hoechst 33342 (10 μg/mL) when required for confocal imaging. The cells were washed in DBPS, 1% w/v BSA, 2 mM CaCl₂) before being imaged using 40× magnification on a Nikon A1/HD25 microscope (Nikon Instruments, Inc., Melville, NY). Processing of fluorescence microscopy images was performed using FIJI (National Institutes of Health) 33.

### Flow Cytometry

Cell lines were suspended as 1,000,000 cells/mL in cold in DBPS, 1% w/v BSA, 2 mM CaCl₂) (supplemented with 50 mM EDTA for adherent cell cultures), filtered through a cell strainer and kept on ice. Flow cytometry data acquisition was obtained on a BD® LSR II Flow Cytomcter and results analyzed using FlowJo™ 10.8.1. Single color controls were included for compensation adjustments.

### General Statistical Analysis

All statistical analyses were conducted in GraphPad Prism v.9.2.0.

### Single Population Experiments

Suspension cell lines: The K562 cell lines were cultured were individually. For an experiment, the culture of interest was spun down and counted to estimate the cell concentration. Then approximately 200,000 cells were isolated, washed twice with DPBS, 1% w/v BSA, 2 mM CaCl₂) and suspended in 0.4 mL of the buffer. To the sample was added the specified SMART-SpyCatcher at the specified concentration by adding each individual component (SpyN and SpyC) from stock solutions. The sample was incubated for 2 hr, 37° C., 5% CO₂ to allow for actuation. The sample was then cooled at room temperature for 10 min before adding the specified SpyTag003 conjugate at 100 nM. The sample was incubated for 20 min at room temperature, in the dark to allow reaction between SpyTag003-SpyCatcher003. The cells were then washed twice with cold DPBS, 1% w/v BSA, 2 mM CaCl₂) and incubated on ice until further analysis by confocal microscopy or flow-cytometry. Samples subjected to SDS-PAGE/western blotting were washed twice with DPBS alone to remove excess BSA. The control experiments examining the mechanism of action in Extended FIG. 3a-c included the following steps prior to the addition of SpyTag003-AF594: 1) addition of competing DARPins (100 nM) blocking SMART-SpyCatcher binding, 2) addition of Cys1-alkylated eNrdJ-1N^{cage} component unable to perform splicing, or 3) pre-addition of unlabeled SpyTag003 (500 nM) to block reaction with SpyTag003-AF594. In reactions with SpyTag003D117A-AF594 the mutation D117A was introduced, which disables the formation of an isopeptide bond with SpyCatcher003.
Adherent cell lines: The various adherent cell lines were cultured individually. Plated cells were lifted using trypsin, washed with complete medium and counted to estimate the cell concentration. Then 200,000 cells were seeded in a 24 well plate format and allowed 24 hr recovery and attachment in complete media. The assay protocol for SMART-SpyCatcher actuation and SpyTag003 recruitment follows that outlined above for the suspension cell lines. Post-treatment, the adherent cells were washed twice with DPBS, 1% w/v BSA, 2 mM CaCl₂) and then lifted with a non-enzymatic solution of DPBS, 1% w/v BSA, 50 mM EDTA and incubated on ice before analysis by flow-cytometry. For SDS-PAGE/western blotting cells were lifted as described and then washed with DPBS alone to remove excess BSA.

### Mixed-Population Experiments

Suspension cell lines: The K562 cell lines were cultured individually. For an experiment, the cultures of interest were spun down and counted to estimate the cell concentrations. Two distinct mixed populations were then generated:
Mixed population 1
   K562^{EpCAMlo} (wildtype cell line)
   K562^{EGFR+/EpCAMlo}
   K562^{HER2+/EpCAMlo}
   K562^{HER2+/EGFR+/EpCAMlo}
Mixed population 2
   K562^{EpCAMlo} (wildtype cell line)
   K562^{EGFR+/EpCAMlo}
   K562^{HER2+/EpCAMhi}
   K562^{HER2+/EGFR+/EpCAMhi.}

The mixed populations were made by aliquoting equal numbers of each specified cell line into a common reaction vessel. Briefly, 50,000 cells/reaction of each of the specified four cell lines were pipetted into a test tube thereby totaling 200,000 cells/reaction. Mixed population 1 and mixed population 2 were then assayed as two separate samples to which SMART-SpyCatcher was added at the specified concentration by adding each individual component (SpyN and SpyC) from stock solutions. In experiments involving NOT gating the decoy was added at 100 nM just before the addition of SMART-SpyCatcher. The sample was incubated for 2 hr, 37° C., 5% CO₂ to allow for actuation. The sample was then cooled at room temperature for 10 min before adding the specified SpyTag003 conjugate at 100 nM. The sample was incubated for 20 min at room temperature, in the dark to allow reaction between SpyTag003-SpyCatcher003. The cells were then washed twice with cold DPBS, 1% w/v BSA, 2 mM CaCl₂) and incubated on ice until further analysis by confocal microscopy or flow-cytometry.

When appropriate for data representation, the individual data sets from the two distinct mixed populations were combined into one common bar graph.

Adherent cell lines: The three adherent cell lines MCF-10a, MCF7, and Sk-br-3 were cultured individually. Each cell line was lifted with a non-enzymatic solution of DPBS, 1% w/v BSA, 50 mM EDTA and counted to estimate the cell concentrations. MCF-10a was then mixed in equal numbers with either MCF-7 or Sk-br-3, and the two mixed populations washed with either complete DMEM medium or complete McCoy's 5a Medium Modified medium respectively, before being aliquoted as 200,000 cells/well in a 24 well plate. The cells were incubated for 6 hours at 37° C., 5% CO₂. The assay protocol for SMART-SpyCatcher actuation and SpyTag003 recruitment follows that outlined above for the suspension cell lines. Post-treatment, the adherent cells were washed twice with DPBS, 1% w/v BSA, 2 mM CaCl₂) and then lifted with a non-enzymatic solution of DPBS, 1% w/v BSA, 50 mM EDTA and incubated on ice before analysis by flow-cytometry.

### Cell Phenotyping

The various adherent cell lines were cultured individually. Plated cells were lifted with trypsin, washed with complete medium and counted to estimate the cell concentration. Then 200.000 cells were seeded in a 24 well plate format and allowed 24 hr recovery and attachment in complete media. The cells were then washed with DBPS, 1% w/v BSA, 2 mM CaCl₂) and incubated 30 min at room temperature in said buffer supplemented with 100 nM Alexa Fluor 594 DARPin conjugate targeting either of the three antigens HER2, EGFR, or EpCAM. The cells were washed twice with DBPS, 1% w/v BSA, 2 mM CaCl₂) before being lifted with a non-enzymatic solution of DPBS, 1% w/v BSA, 50 mM EDTA and incubated on ice until further analysis by flow-cytometry.

### Anti-DNP Monoclonal IgG Recruitment Assay

The sample was prepared following the assay protocol detailed for experiments involving a single population or mixed populations of suspension cells described above. After cell sample preparation and incubation with SMART-SpyCatcher a SpyTag003 2,4-dinitrolphenol conjugate (SpyTag003-DNP) was used in the reaction between SpyTag003-SpyCatcher003. Briefly, the sample was incubated at room temperature for 10 min before adding the SpyTag003-DNP at 100 nM. The sample was incubated for 20 min at room temperature, in the dark to allow reaction between SpyTag003-SpyCatcher003. The cells were then washed twice with DPBS, 1% w/v BSA, 2 mM CaCl₂) and suspended in the buffer supplemented with primary anti-DNP IgG antibody (1:1000). After additional incubation for 30 min at room temperature any excess anti-DNP IgG antibody was washed away with DBPS, 1% w/v BSA, 2 mM CaCl₂) and the cells suspended in buffer supplemented with Goat anti-Rabbit IgG antibody Alexa Fluor 594 conjugate (1:4.000). After additional incubation for 30 min at room temperature excess Goat anti-rabbit IgG antibody was washed away with DBPS, 1% w/v BSA, 2 mM CaCl₂) and the cells suspended in buffer before being imaged by confocal microscopy or analyzed by flow cytometry. The control experiments validating that anti-DNP IgG engagement was caused by SpyTag003-DNP recruitment included the following steps prior to addition of SpyTag003-DNP: by 1) addition of competing DARPins (100 nM) blocking SMART-SpyCatcher binding, 2) addition of Cys1-alkylated eNrdJ-1N^{cage} component unable to perform splicing, or 3) addition of unlabeled SpyTag003 to block reaction with the SpyTag003-DNP variant.

### NeutrAvidin Rhodamine Red-X Recruitment Assay

The sample was prepared following the assay protocol detailed for single population or mixed populations of suspension cells described above. After cell sample preparation and incubation with SMART-SpyCatcher, a SpyTag003 biotin conjugate (SpyTag003-Biotin) was used in the reaction between SpyTag003-SpyCatcher003. Briefly, the sample was incubated at room temperature for 10 min before adding the SpyTag003-Biotin at 100 nM. The sample was incubated for 20 min at room temperature in the dark to allow reaction between SpyTag003-SpyCatcher003. The cells were then washed twice with cold DPBS, 1% w/v BSA, 2 mM CaCl₂) before addition of NeutrAvidin Rhodamine Red-X (1:500) and an additional incubation for 30 min at room temperature. Excess NeutrAvidin Rhodamine Red-X was washed away, and the cells suspended in DBPS, 1% w/v BSA, 2 mM CaCl2 before being imaged by confocal microscopy or analyzed by flow cytometry. For the experiment studying the intake of NeutrAvidin Rhodamine Red-X the sample was imaged immediately and after an addition incubation period of 4 hr at the specified temperature.

### Cell Depletion Assay

Mixed K562 population depletion: The sample was prepared following the assay protocol detailed for mixed populations of suspension cells described above. After cell sample preparation and incubation with SMART-SpyCatcher a SpyTag003 biotin conjugate (SpyTag003-Biotin) was used in the reaction between SpyTag003-SpyCatcher003. Briefly, the sample was incubated at room temperature for 10 min before adding the SpyTag003-Biotin to 100 nM. After another 30 min at room temperature to allow for SpyTag003-SpyCatcher003 reaction, the cells were washed with and suspended in 1.6 mL complete RPMI-1640 medium. For each condition and replicate tested was then withdrawn 100 μL of 12,500 cells, aliquoted in a 96 well plate format. An additional 100 μL complete RPMI-1640 media was added to each well, supplemented with 40 nM Streptavidin-Saporin when indicated giving a final concentration of 20 nM of the toxin conjugate. The cells were then cultured for 72 hr for the single dose regimen. For the two-dose regiment the cells were cultured for 24 hr before being subjected to the treatment described above a second time, and then cultured for an additional 72 hr. To sample for variations in the mixed cell population composition, the sample was carefully pipetted into a FACS tube and analyzed by flow cytometry.

Subpopulation percentages obtained from the flow cytometry analysis were normalized using the following formula:

$$Viability ( \% ) = \frac{{X}_{1}}{{X}_{0} \frac{{WT}_{1}}{{WT}_{0}}} \cdot 100 \%$$

where X₀ is the percentage of the specific subpopulation in the negative untreated sample, X₁ is the percentage of the specific subpopulation the relevant sample, WT₀ is the percentage of the wildtype subpopulation in the negative untreated sample, and WT₁ is the percentage of the wildtype subpopulation in the relevant sample. The derived percentage is taken as the viability of the specified subpopulation.
Single A431 population depletion: For the A431 cell depletion assay the cultured cells were lifted with trypsin, counted, and then seeded as 5,000 cells per well in a 96 well format before further culturing for 24 hr at 37° C., 5% CO₂. Then the cells were washed with DPBS, 1% w/v BSA, 2 mM CaCl₂) and incubated with the indicated SMART-SpyCatcher 100 nM. Following 2 hr at 37° C., 5% CO₂, the sample was incubated for 10 min at room temperature. SpyTag003-Biotin was added to 100 nM, and the sample incubated for another 30 min at room temperature. The cells were gently washed with complete DMEM medium before the addition of 200 μL DMEM medium supplemented with 20 nM Streptavidin-Saporin conjugate when indicated. The cells were cultured 24 hr at 37° C., 5% CO₂, before the complete treatment described above was repeated to give a two-dose regimen. Finally, the cells were cultured for an additional 72 hr. Each well was then washed twice with complete DMEM medium and 200 μL complete DMEM medium added. An XTT cell viability assay was used to quantify the metabolic activity of each sample according to the protocol prescribed by the manufacturer. The conversion of XTT to formazan was measured on a SpectraMax® iD5 Multi-Mode Microplate Reader (Molecular Devices) using well-scan mode at 465 nm at room temperature. The obtained absorbance values were normalized to the negative untreated control.

### APEX2 Labeling

The sample was prepared following the assay protocol detailed for single populations described above. After cell sample preparation and incubation with SMART-SpyCatcher a SpyTag003-APEX2 fusion was used in the reaction between SpyTag003-SpyCatcher003. Briefly, the cell sample was incubated at room temperature for 10 min before adding the SpyTag003-APEX2 at 300 nM. After another 30 min at room temperature to allow for SpyTag003-SpyCatcher003 reaction, the cells were washed twice with DPBS alone. Then 1 mL freshly prepared DPBS, 250 μM biotin-phenol was added. To activate APEX2 labeling 10 μL freshly prepared DPBS, 100 mM H₂O₂ was added, and the reaction allowed at room temperature under gentle swirling for the time indicated. The reaction was quenched by the addition of 200 μL DPBS, 5 mM Trolox, 10 mM sodium ascorbate and further washed twice with 1 mL of the quencher buffer. The cells were lifted (when necessary) by the addition of 1 mL DPBS, 50 mM EDTA, pelleted and suspended in 200 μL-500 μL 50 mM Tris-HCl (pH 8.0), 150 mM NaCl, 1% v/v NP-40, 0.5% v/v sodium deoxycholate, 0.1% w/v sodium dodecyl sulfate supplemented with 1×Halt™ Protease Inhibitor Cocktail and 1 mM PMSF, then sonicated briefly and centrifuged for 20 min at 15,000 g, 4° C. The supernatant was isolated and analyzed by SDS-PAGE and western blotting. Image quantification was performed using FIJI (National Institutes of Health).

TABLE 3
Crystallization data collection and refinement statistics. Highest-
resolution shell shown in parentheses. R_{free} represents the R-factor
calculated from 5% of the reflections not used during refinement.
Data collection

| | |
|---|---|
| Wavelength (Å) | 0.97242 |
| Space group | P 21 21 21 |

Cell dimensions

| | |
|---|---|
| a, b, c (Å) | 48.79, 84.38, 174.71 |
| β (degree) | 90 |
| Resolution (Å) | 29.12-1.95 |
| Rpim | 0.053 (0.352) |
| I/σI | 1.66 |
| CC1/2 (%) | 99.7 (80.8) |
| Completeness (%) | 99.8 (98.1) |
| Anomalous completeness (%) | 98.4 (95.6) |
| Redundancy (%) | 6.8 (6.6) |
| Anomalous redundancy (%) | 3.5 (3.1) |
| Wilson B factor (Å²) | 26.5 |

Refinement

| | |
|---|---|
| Resolution (Å) | 1.95 |
| Reflections/unique | 362053/53184 |
| R_{work}/R_{free} | 0.1801/0.2210 |

No. atoms/ions

| | |
|---|---|
| Protein | 4800 |
| Other | 423 |
| Average B-factor (Å²) | 36 |

R.M.S. deviations

| | |
|---|---|
| Bond lengths (Å) | 0.006 |
| Bond angles (degree) | 0.802 |

Ramachandran (%)

| | |
|---|---|
| Favored | 98.2 |
| Allowed | 1.8 |

TABLE 4
Antibodies used for western blotting. Tabulated are epitopes
and antibodies used in the study. All dilutions were done in
TBS-T (25 mM Tris-HCl (pH 7.7), 150 mM NaCl, 0.1% v/v Tween-
20) supplemented with 3% w/v BSA and 0.02% w/v sodium azide.

| Epitope | Antibody | Vendor (product) | Dilution |
|---|---|---|---|
| FLAG | Rabbit anti-FLAG | Sigma (F7425) | 1:2,000 |
| Myc | Mouse anti-Myc | CST (2276S) | 1:2,000 |
| HA | Mouse anti-HA | Invitrogen (26183) | 1:2,000 |
| HER2 | Rabbit anti-HER2 | CST (4290) | 1:1,000 |
| EGFR | Rabbit anti-EGFR | CST (4267) | 1:1,000 |
| EpCAM | Mouse anti-EpCAM | Abcam (ab216136) | 1:2,000 |
| Dinitrophenol | Rabbit anti-DNP | CST (14681) | 1:10,000 |
| Rabbit IgG | Goat Anti-Rabbit IgG H&L | Abcam (ab150080) | 1:10,000 |
| | (Alexa Fluor ® 594) | | |
| Rabbit IgG | IRDye 680RD Goat anti-Rabbit | LI-COR (926- | 1:10,000 |
| | IgG (H + L) | 68071) | |
| Rabbit IgG | IRDye 800CW Goat anti-Rabbit | LI-COR (926- | 1:10,000 |
| | IgG (H + L) | 32211) | |
| Mouse IgG | IRDye 680RD Goat anti-Mouse | LI-COR (926- | 1:10,000 |
| | IgG (H + L) | 68070) | |
| Mouse IgG | IRDye 800CW Goat anti-Mouse | LI-COR (926- | 1:10,000 |
| | IgG (H + L) | 32210) | |
| Biotin | IRDye 800CW Streptavidin | LI-COR (926- | 1:10,000 |
| | | 32230) | |

TABLE 5
Mammalian cell culturing. All media were prepared with
100 U/mL penicillin/streptomycin and stored at 4° C.

| Cell line | Medium | FBS | Supplements |
|---|---|---|---|
| K562^{EpCAMlo} (wildtype) | RPMI-1640 | 5% | 25 mM HEPES |
| K562^{HER2+} | RPMI-1640 | 5% | 25 mM HEPES |
| K562^{EGFR+} | RPMI-1640 | 5% | 25 mM HEPES |
| K562^{HER2+/EGFR+} | RPMI-1640 | 5% | 25 mM HEPES |
| K562^{HER2+/EpCAMhi} | RPMI-1640 | 5% | 25 mM HEPES |
| K562^{HER2+/EGFR+/EpCAMhi} | RPMI-1640 | 5% | 25 mM HEPES |
| OE19 | RPMI-1640 | 10% | — |
| A431 | DMEM | 10% | — |
| MCF-7 | DMEM | 10% | — |
| HCT-116 | McCoy's 5a Medium | 10% | — |
| | Modified | | |
| SK-BR-3 | McCoy's 5a Medium | 10% | — |
| | Modified | | |
| A549 | F-12K Medium | 10% | — |
| LoVo | F-12K Medium | 10% | — |
| MCF-10a | Mammary Epithelial | 5% | Bovine Pituitary Extract |
| | Cell Growth Medium | | (0.004 ml/ml), Epidermal |
| | | | Growth Factor (recombinant |
| | | | human, 10 ng/ml), insulin |
| | | | (recombinant human, |
| | | | 5 μg/ml), hydrocortisone |
| | | | (0.5 μg/ml) |

### ASPECTS OF THE INVENTION

Aspect 1. A logic-gated protein actuator system, comprising:
a mixture comprising
   a first component comprising a first caged split intein fragment fused to a first protein-of-interest fragment and fused to a first antigen-binding domain configured to bind to a first surface antigen; and
   a second component comprising a second caged split intein fragment fused to a second protein-of-interest fragment and fused to a second antigen-binding domain configured to bind to a second surface antigen,
wherein the first caged split intein fragment and the second caged split intein fragment are capable of undergoing protein trans-splicing to form a protein of interest from the first protein-of-interest fragment and the second protein-of-interest fragment when the first antigen-binding domain and the second antigen-binding domain are proximal, and
wherein the first caged split intein fragment and the second caged split intein fragment are an actuator.

Aspect 2. The logic-gated protein actuator system of Aspect 1,
wherein the first surface antigen and the second surface antigen are different,
wherein the mixture does not comprise a third component comprising the first caged split intein fragment fused to the first protein-of-interest fragment and fused to the second antigen-binding domain,
wherein the mixture does not comprise a fourth component comprising the second caged split intein fragment fused to the second protein-of-interest fragment and fused to the first antigen-binding domain, and
wherein the logic-gated protein actuator system is a first-surface-antigen-AND-second-surface-antigen logic gate.

Aspect 3. The logic-gated protein actuator system of any one of Aspects 1 and 2,
wherein the mixture comprises a fifth component comprising the second caged split intcin fragment fused to the second protein-of-interest fragment and fused to a third antigen-binding domain configured to bind to a third surface antigen.
wherein the third surface antigen is different from the first surface antigen and from the second surface antigen,
wherein the first caged split intein fragment and the second caged split intein fragment are capable of undergoing protein trans-splicing to form the protein of interest from the first protein-of-interest fragment and the second protein-of-interest fragment when the third antigen-binding domain and the first antigen-binding domain are proximal,
wherein the logic-gated protein actuator system is a first-surface-antigen-AND-either-second-surface-antigen-OR-third-surface-antigen logic gate.

Aspect 4. The logic-gated protein actuator system of Aspect 3,
wherein the mixture comprises at least one additional component,
wherein each additional component comprises the second caged split intein fragment fused to the second protein-of-interest fragment and fused to an additional antigen-binding domain configured to bind to an additional surface antigen.
wherein each additional surface antigen of each additional component is different from the first surface antigen, the second surface antigen, the third surface antigen, and each other additional surface antigen,
wherein the first caged split intein fragment and the second caged split intein fragment are capable of undergoing protein trans-splicing to form the protein of interest from the first protein-of-interest fragment and the second protein-of-interest fragment when the additional antigen-binding domain is proximal to the third antigen-binding domain, the second antigen-binding domain, the first antigen-binding domain, or any other additional binding domain,
wherein the logic-gated protein actuator system is a first-surface-antigen-AND-either-second-surface-antigen-OR-third-surface-antigen-OR-additional-surface-antigen(s) logic gate.

Aspect 5. The logic-gated protein actuator system of any one of Aspects 1 and 2,
wherein the mixture comprises a fifth component comprising the second caged split intein fragment fused to a modified second protein-of-interest fragment and fused to a third antigen-binding domain configured to bind to a third surface antigen,
wherein the third surface antigen is different from the first surface antigen and from the second surface antigen,
wherein the first caged split intein fragment and the second caged split intein fragment of the fifth component are capable of undergoing protein trans-splicing to form a defective protein of interest from the first protein-of-interest fragment and the modified second protein-of-interest fragment when the third antigen-binding domain and the first antigen-binding domain are proximal,
wherein the defective protein of interest is different from the protein of interest,
wherein the logic-gated protein actuator system is a first-surface-antigen-AND-second-surface-antigen-NOT-third-surface-antigen logic gate.

Aspect 6. The logic-gated protein actuator system of Aspect 5,
wherein the mixture comprises a sixth component comprising the second caged split intein fragment fused to the modified second protein-of-interest fragment and fused to a fourth antigen-binding domain configured to bind to a fourth surface antigen,
wherein the fourth surface antigen is different from the first surface antigen, from the second surface antigen, and from the third surface antigen,
wherein the first caged split intein fragment and the second caged split intein fragment of the sixth component are capable of undergoing protein trans-splicing to form the defective protein of interest from the first protein-of-interest fragment and the modified second protein-of-interest fragment when the fourth antigen-binding domain and the first antigen-binding domain are proximal,
wherein the logic-gated protein actuator system is a first-surface-antigen-AND-second-surface-antigen-NOT-third-surface-antigen-NOT-fourth-surface-antigen logic gate.

Aspect 7. The logic-gated protein actuator system of Aspect 6,
wherein the mixture comprises at least one additional component comprising the second caged split intcin fragment fused to the modified second protein-of-interest fragment and fused to an additional antigen-binding domain configured to bind to an additional surface antigen,
wherein the additional surface antigen is different from the first surface antigen, from the second surface antigen, from the third surface antigen, and from the fourth surface antigen,
wherein the first caged split intein fragment and the second caged split intein fragment of the additional component are capable of undergoing protein trans-splicing to form the defective protein of interest from the first protein-of-interest fragment and the modified second protein-of-interest fragment when the additional antigen-binding domain and the first antigen-binding domain are proximal,
wherein the logic-gated protein actuator system is a first-surface-antigen-AND-second-surface-antigen-NOT-third-surface-antigen-NOT-fourth-surface-antigen-NOT-additional-surface-antigen logic gate.

Aspect 8. The logic-gated protein actuator system of Aspect 5,
wherein the first surface antigen and the second surface antigen are identical and
wherein the logic-gated protein actuator system is a first-surface-antigen-NOT-third-surface-antigen logic gate.

Aspect 9. The logic-gated protein actuator system of Aspect 6,
wherein the first surface antigen and the second surface antigen are identical and
wherein the logic-gated protein actuator system is a first-surface-antigen-AND-[third-surface-antigen-NOR-fourth-surface-antigen] logic gate.

Aspect 10. The logic-gated protein actuator system of Aspect 1,
wherein the first surface antigen and the second surface antigen are different,
wherein the mixture comprises a third component comprising the first caged split intein fragment fused to the first protein-of-interest fragment and fused to the second antigen-binding domain,
wherein the mixture comprises a fourth component comprising the second caged split intcin fragment fused to the second protein-of-interest fragment and fused to the first antigen-binding domain,
wherein the first caged split intein fragment and the second caged split intein fragment are capable of undergoing protein trans-splicing to reconstitute the protein of interest from the first protein-of-interest fragment and the second protein-of-interest fragment when two first antigen-binding domains are proximal,
wherein the first caged split intein fragment and the second caged split intein fragment are capable of undergoing protein trans-splicing to reconstitute the protein of interest from the first protein-of-interest fragment and the second protein-of-interest fragment when two second antigen-binding domains are proximal, and
wherein the logic-gated protein actuator system is a first-surface-antigen-OR-second-surface-antigen logic gate.

Aspect 11. The logic-gated protein actuator system of Aspect 10,
wherein the mixture comprises a fifth component comprising the first caged split intein fragment fused to the first protein-of-interest fragment and fused to a third antigen-binding domain,
wherein the mixture comprises a sixth component comprising the second caged split intein fragment fused to the second protein-of-interest fragment and fused to a fourth antigen-binding domain,
wherein the third antigen-binding domain is different from the second antigen-binding domain and from the first antigen-binding domain,
wherein the first caged split intein fragment and the second caged split intein fragment are capable of undergoing protein trans-splicing to reconstitute the protein of interest from the first protein-of-interest fragment and the second protein-of-interest fragment when the third antigen-binding domain is proximal to the first antigen-binding domain or the second antigen-binding domain or two third antigen-binding domains are proximal, and
wherein the logic-gated protein actuator system is a first-surface-antigen-OR-second-

Aspect 12. The logic-gated protein actuator system of Aspect 11,
wherein the mixture comprises at least one additional pair of components,
wherein the first part of the additional pair of components comprises the first caged split intein fragment fused to the first protein-of-interest fragment and fused to an additional antigen-binding domain configured to bind to an additional surface antigen,
wherein the second part of the additional pair of components comprises the second caged split intein fragment fused to the second protein-of-interest fragment and fused to the additional antigen-binding domain,
wherein each additional surface antigen is different from the first surface antigen, the second surface antigen, the third surface antigen, and each other additional surface antigen,
wherein the first caged split intein fragment and the second caged split intein fragment are capable of undergoing protein trans-splicing to form the protein of interest from the first protein-of-interest fragment and the second protein-of-interest fragment when the additional antigen-binding domain is proximal to the third antigen-binding domain, the second antigen-binding domain, the first antigen-binding domain, or any other additional binding domain, and
wherein the logic-gated protein actuator system is a first-surface-antigen-OR-second-surface-antigen-OR-third-surface-antigen-OR-additional-surface-antigen(s) logic gate.

Aspect 13. The logic-gated protein actuator system of any one of Aspects 1 through 12,
wherein the first caged split intein fragment is eNrdJ-1N^{cage}-K114AK116A and
wherein the second caged split intein fragment is eNrdJ-1C^{cage}.

Aspect 14. The logic-gated protein actuator system of any one of Aspects 1 through 12,
wherein the first caged split intein fragment is eNrdJ-1C^{cage} and
wherein the second caged split intein fragment is eNrdJ-1N^{cage}-K114AK116A.

Aspect 15. The logic-gated protein actuator system of any one of Aspects 1 through 12,
wherein the pair of the first caged split intein fragment and the second caged split intein fragment are selected from the group of pairs consisting of eNrdJ-1N^{cage} & eNrdJ-1C^{cage}, NrdJ-IN^{cage} & NrdJ-1C^{cage}, NpuN^{cage} & NpuC^{cage}, Gp41-1N^{cage} & Gp41-1C^{cage}. Gp41-8N^{cage} & Gp41-8C^{cage}, eNrdJ-1C^{cage} & eNrdJ-1N^{cage}, NrdJ-1C^{cage} & NrdJ-1N^{cage}, NpuC^{cage} & NpuN^{cage}, Gp41-1C^{cage} & Gp41-1N^{cage}, and Gp41-8C^{cage} & Gp41-8N^{cage}.

Aspect 16. The logic-gated protein actuator system of any one of Aspects 1 through 15,
wherein the protein of interest comprises a toxin.

Aspect 17. The logic-gated protein actuator system of any one of Aspects 1 through 15,
wherein the protein of interest comprises an imaging agent.

Aspect 18. The logic-gated protein actuator system of any one of Aspects 1 through 15,
wherein the protein of interest comprises an antibody epitope.

Aspect 19. The logic-gated protein actuator system of any one of Aspects 1 through 15,
wherein the protein of interest comprises an antigen or antigen fragment to which an antibody or an antibody paratope or a T-cell or a T-cell receptor can bind or a protein, protein fragment, antigen, antibody, or antigen-antibody complex to which a complement protein can bind.

Claim 20. The logic-gated protein actuator system of any one of Aspects 1 through 15,
wherein the protein of interest comprises an enzyme.

Aspect 21. The logic-gated protein actuator system of any one of Aspects 1 through 15,
wherein the protein of interest is capable of binding a toxin.

Aspect 22. The logic-gated protein actuator system of any one of Aspects 1 through 15,
wherein the protein of interest is capable of binding an imaging agent.

Aspect 23. The logic-gated protein actuator system of any one of Aspects 1 through 15,
wherein the protein of interest is capable of binding an antibody epitope.

Aspect 24. The logic-gated protein actuator system of any one of Aspects 1 through 15,
wherein the protein of interest is capable of binding an antigen or an antigen fragment to which an antibody or an antibody paratope or a T-cell or a T-cell receptor can bind or a protein, protein fragment, antigen, antibody, or antigen-antibody complex to which a complement protein can bind.

Aspect 25. The logic-gated protein actuator system of any one of Aspects 1 through 15,
wherein the protein of interest is capable of binding an enzyme.

Aspect 26. The logic-gated protein actuator system of any one of Aspects 1 through 15,
wherein the protein of interest is SpyCatcher003.

Aspect 27. The logic-gated protein actuator system of any one of Aspects 1 through 15,
wherein the first protein of interest fragment is SpyN¹⁻⁷³ and
wherein the second protein of interest fragment is SpyC^{74-113.}

Aspect 28. The logic-gated protein actuator system of any one of Aspects 1 through 15,
wherein the first protein of interest fragment is SpyC⁷⁴⁻¹¹³ and
wherein the second protein of interest fragment is SpyN^{1-73.}

Aspect 29. The logic-gated protein actuator system of any one of Aspects 1 through 15,
wherein the pair of the first protein-of-interest fragment and the second protein-of-interest fragment are selected from the group of pairs consisting of SpyN¹⁻²⁴ & SpyC²⁵⁻¹¹³ SpyN¹⁻⁴² & SpyC⁴³⁻¹¹³, SpyN¹⁻⁵⁵ & SpyC⁵⁶⁻¹¹³. SpyN¹⁻⁸² & SpyC⁸³⁻¹¹³, SpyN¹⁻⁹⁰ & SpyC⁹¹⁻¹¹³. SpyC²⁵⁻¹¹³ & SpyN¹⁻²⁴, SpyC⁴³⁻¹¹³ & SpyN¹⁻⁴², SpyC⁵⁶⁻¹¹³ & SpyN¹⁻⁵⁵, SpyC⁸³⁻¹¹³ & SpyN¹⁻⁸², and SpyC⁹¹⁻¹¹³ & SpyN^{1-90.}

Aspect 30. The logic-gated protein actuator system of any one of Aspects 1 through 29, wherein the mixture further comprises a SpyTag003-biotin conjugate.

Aspect 31. The logic-gated protein actuator system of any one of Aspects 1 through 29, wherein the mixture further comprises a SpyTag003-biotin conjugate and a Streptavidin-Saporin conjugate.

Aspect 32. The logic-gated protein actuator system of any one of Aspects 1 through 29, wherein the mixture further comprises a SpyTag003-AF594 conjugate.

Aspect 33. The logic-gated protein actuator system of any one of Aspects 1 through 29, wherein the mixture further comprises a SpyTag003-APEX2 conjugate.

Aspect 34. The logic-gated protein actuator system of any one of Aspects 1 through 33,
wherein the first surface antigen is selected from the group consisting of HER2, EGFR, and EpCAM and
wherein the second surface antigen is selected from the group consisting of HER2, EGFR, and EpCAM.

Aspect 35. The logic-gated protein actuator system of any one of Aspects 1, 3 through 9, 11, and 12,
wherein the first surface antigen is selected from the group consisting of HER2, EGFR, and EpCAM and
wherein the second surface antigen is selected from the group consisting of HER2, EGFR, and EpCAM,
wherein the third surface antigen is selected from the group consisting of HER2, EGFR, and EpCAM.

Aspect 36. The logic-gated protein actuator system of any one of Aspects 1 through 33,
wherein each surface antigen is selected from the group consisting of HER2, EGFR, and EpCAM.

Aspect 37. A method of conditionally forming a protein of interest, comprising:
providing a mixture comprising
   a first component comprising a first caged split intein fragment fused to a first protein-of-interest fragment and fused to a first antigen-binding domain configured to bind to a first surface antigen; and
   a second component comprising a second caged split intein fragment fused to a second protein-of-interest fragment and fused to a second antigen-binding domain configured to bind to a second surface antigen,
bringing the mixture into contact with a cell having a surface, and
when the first surface antigen and the second surface antigen are proximal on the surface of the cell, allowing the first antigen-binding domain to bind to the first surface antigen and the second antigen-binding domain to bind to the second surface antigen, and allowing the first caged split intein fragment and the second caged split intein fragment to undergo protein trans-splicing to form a protein of interest from the first protein-of-interest fragment and the second protein-of-interest fragment,
wherein the first caged split intein fragment and the second caged split intein fragment are an actuator.

Aspect 38. The method of Aspect 37, further comprising
allowing the protein of interest to bind a toxin, and
allowing the toxin to damage or kill the cell.

Aspect 39. Treating a cancer by the method of Aspect 38,
further comprising administering the mixture and the toxin to a patient,
wherein the cell is a cancerous cell.

Aspect 40. The method of Aspect 37, further comprising
allowing the protein of interest to bind an imaging agent, and
using the imaging agent to image the cell.

Aspect 41. Diagnosing a hyperproliferative disorder by the method of Aspect 40,
further comprising administering the mixture and the imaging agent to a patient,
wherein the cell is a hyperproliferative cell.

Aspect 42. The method of Aspect 37,
wherein the protein of interest comprises a toxin and
further comprising allowing the toxin to damage or kill the cell.

Aspect 43. The method of Aspect 37,
wherein the protein of interest comprises an imaging agent and
further comprising using the imaging agent to image the cell.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### REFERENCES

1. Bausch-Fluck, D. et al. The in silico human surfaccome. Proc. Natl. Acad. Sci. USA 115 E10988-E10997 (2018)
2. Annenfelser. R. et al. Discriminatory Power of Combinatorial Antigen Recognition in Cancer T Cell Therapies. 11, 215-228 (2020)
3. Hu, Z. et al. The Cancer Surfaceome Atlas integrates-genomic functional and drug response data to identify actionable targets. Nat. Cancer. 2, 1406-1422 (2021)
4. Kichloo, A. et al. Systemic adverse effects and toxicities associated with immunotherapy: A review 12, 150-163 (2021)
5. Tarantino, P. et al, Optimizing the safety of antibody-drug conjugates for patients with solid tumors. Nat. Rev. Clin. Oncol. 20, 558-576 (2023)
6. Mackay, M. et al. The therapeutic landscape for cells engineered with chimeric antigen receptors. Nat. Biotechnol. 38, 233-244 (2020)
7. Savanur, M. A. et al, Implementing Logic Gates for Safer Immunotherapy of Cancer. Front Immunol. 12, 780399 (2021)
8. Lajoie, M. J. et al. Designed protein logic to target cells with precise combinations of surface antigens. Science. 369, 1637-1643 (2020)
9. Quijano-Rubio, A. et al. A split, conditionally active mimetic of IL-2 reduces the toxicity of systemic cytokine therapy. Nat. Biotechnol. 41, 532-540 (2023)
10. Gunnoo, S. B. et al Creation of a gated antibody as a conditionally functional synthetic protein. Nat. Commun. 5, 4388 (2014)
11. Oostinidie, S. C. et al. Logic-gated antibody pairs that selectively act on cells co-expressing two antigens. Nat. Biotechnol. 40, 1509-1519 (2022)
12. Pedram, K. et al. Design of a mucin-selective protease for targeted degradation of cancer-associated mucins. bioRxiv preprint (2020)
13. Gray, M. et al. Targeted glycan degradation potentiates the anticancer immune response in vivo. Nat. Chem. Biol. 16, 1376-1384 (2020)
14. Porta, C. et al. Ranpirnase and its potential for the treatment of unresectable malignant mesothelioma. Biol.: Targets Ther. 2, 601-609 (2008)
15. Huynh, V. T. et al. Asparaginase Toxicities: Identification and Management in Patients With Acute Lymphoblawith Leukemia. Clin. J. Oncol. Nurs. 21, E248-E259 (2017)
16. Shah, N. et al. Inteins: nature's gift to protein chemists. Chem. Sci. 5, 446-461 (2014)
17. Carvajal-Vallcjos, P. et al. Unprecedented Rates and Efficiencies Revealed for New Natural Split Inteins from Metagenomic Sources. J. Biol. Chem. 287, 28686-28696 (2012)
18. Gramespacher, J. A. et al. Intein Zymogens: Conditional Assembly and Splicing of Split Inteins via Targeted Proteolysis. J. Am. Chem. Soc. 139, 8074-8077 (2017)
19. Gramespacher, J. A. et al. Proximity Induced Splicing Utilizing Caged Split Inteins. J. Am. Chem. Soc. 141, 13708-13712 (2019)
20. Keeble, A. H. et al. Approaching infinite affinity through engineering of peptide-protein interaction. Proc. Natl. Acad. Sci. USA. 116, 26523-26533 (2019)
21. Zahnd, C. et al. A Designed Ankyrin Repeat Protein Evolved to Picomolar Affinity to Her2. J. Mol. Biol. 369, 1015-1028 (2007)
22. Steiner, D. et al. Efficient Selection of DARPIns with Sub-nanomolar Affinities using SRP Phage Display. J. Mol. Biol. 382, 1211-1227 (2008)
23. Stefan, N. et al. DARPins Recognizing the Tumor-Associated Antigen EpCAM Selected by Phage and Ribosome Display and Engineered for Multivalency. J. Mol. Biol. 413, 826-842 (2011)
24. Polito, L. et al. Saporin-S6: A Useful Tool in Cancer Therapy. Toxins. 5, 1698-1722 (2013)
25. Lam, S. S. et al. Directed evolution of APEX2 for electron microscopy and proximity labeling. Nat. Methods 12, 51-54 (2015)
26. Allen, G. et al. Rethinking cancer targeting strategies in the era of smart cell therapeutics. Nat. Rev. Cancer 22, 693-702 (2022)
27. Kabsch, W. et al. Xds. Acta Crystallogr. D Biol. Crystallogr. 66, 125-132 (2010)
28. Winn, M. D. et al. Overview of the CCP4 suite and current developments. Acta Crystallogr. D Biol. Crystallogr. 67(Pt 4): 235-242 (2011)
29. Jumper, J. et al. Highly accurate protein structure prediction with AlphaFold. Nature 596, 583-589 (2021)
30. Varadi, M. et al. AlphaFold Protein Structure Database: massively expanding the structural coverage of protein-sequence space with high-accuracy models. Nucleic Acids Research 50(D1): D439-D444 (2022)
31. Emsley, P. et al. Features and development of Coot. Acta Crystallogr. D Biol. Crystallogr. 66, 486-501 (2010)
32. Liebschner D, et al. Macromolecular structure determination using X-rays, neutrons, and electrons: recent developments in Phenix. Acta Crystallogr. D Struct. Biol. 75, 861-877 (2019)
33. Schindelin, J. et al. Fiji: an open-source platform for biological-image analysis. Nature Methods, 9 (7), 676-682 (2012)

1. A logic-gated protein actuator system, comprising:
a mixture comprising
a first component comprising a first caged split intein fragment fused to a first protein-of-interest fragment and fused to a first antigen-binding domain configured to bind to a first surface antigen; and
a second component comprising a second caged split intein fragment fused to a second protein-of-interest fragment and fused to a second antigen-binding domain configured to bind to a second surface antigen,
wherein the first caged split intein fragment and the second caged split intein fragment are capable of undergoing protein trans-splicing to form a protein of interest from the first protein-of-interest fragment and the second protein-of-interest fragment when the first antigen-binding domain and the second antigen-binding domain are proximal, and
wherein the first caged split intein fragment and the second caged split intein fragment are an actuator.

2. The logic-gated protein actuator system of claim 1,
wherein the first surface antigen and the second surface antigen are different,
wherein the mixture does not comprise a third component comprising the first caged split intein fragment fused to the first protein-of-interest fragment and fused to the second antigen-binding domain,
wherein the mixture does not comprise a fourth component comprising the second caged split intein fragment fused to the second protein-of-interest fragment and fused to the first antigen-binding domain, and
wherein the logic-gated protein actuator system is a first-surface-antigen-AND-second-surface-antigen logic gate.

3. The logic-gated protein actuator system of claim 1,
wherein the mixture comprises a fifth component comprising the second caged split intcin fragment fused to the second protein-of-interest fragment and fused to a third antigen-binding domain configured to bind to a third surface antigen,
wherein the third surface antigen is different from the first surface antigen and from the second surface antigen,
wherein the first caged split intein fragment and the second caged split intein fragment are capable of undergoing protein trans-splicing to form the protein of interest from the first protein-of-interest fragment and the second protein-of-interest fragment when the third antigen-binding domain and the first antigen-binding domain are proximal,
wherein the logic-gated protein actuator system is a first-surface-antigen-AND-either-second-surface-antigen-OR-third-surface-antigen logic gate.

4. The logic-gated protein actuator system of claim 3,
wherein the mixture comprises at least one additional component,
wherein each additional component comprises the second caged split intein fragment fused to the second protein-of-interest fragment and fused to an additional antigen-binding domain configured to bind to an additional surface antigen,
wherein each additional surface antigen of each additional component is different from the first surface antigen, the second surface antigen, the third surface antigen, and each other additional surface antigen,
wherein the first caged split intein fragment and the second caged split intein fragment are capable of undergoing protein trans-splicing to form the protein of interest from the first protein-of-interest fragment and the second protein-of-interest fragment when the additional antigen-binding domain is proximal to the third antigen-binding domain, the second antigen-binding domain, the first antigen-binding domain, or any other additional binding domain,
wherein the logic-gated protein actuator system is a first-surface-antigen-AND-either-second-surface-antigen-OR-third-surface-antigen-OR-additional-surface-antigen(s) logic gate.

5. The logic-gated protein actuator system of claim 1,
wherein the mixture comprises a fifth component comprising the second caged split intein fragment fused to a modified second protein-of-interest fragment and fused to a third antigen-binding domain configured to bind to a third surface antigen,
wherein the third surface antigen is different from the first surface antigen and from the second surface antigen,
wherein the first caged split intein fragment and the second caged split intein fragment of the fifth component are capable of undergoing protein trans-splicing to form a defective protein of interest from the first protein-of-interest fragment and the modified second protein-of-interest fragment when the third antigen-binding domain and the first antigen-binding domain are proximal,
wherein the defective protein of interest is different from the protein of interest,
wherein the logic-gated protein actuator system is a first-surface-antigen-AND-second-surface-antigen-NOT-third-surface-antigen logic gate.

6. The logic-gated protein actuator system of claim 5,
wherein the mixture comprises a sixth component comprising the second caged split intein fragment fused to the modified second protein-of-interest fragment and fused to a fourth antigen-binding domain configured to bind to a fourth surface antigen,
wherein the fourth surface antigen is different from the first surface antigen, from the second surface antigen, and from the third surface antigen,
wherein the first caged split intein fragment and the second caged split intein fragment of the sixth component are capable of undergoing protein trans-splicing to form the defective protein of interest from the first protein-of-interest fragment and the modified second protein-of-interest fragment when the fourth antigen-binding domain and the first antigen-binding domain are proximal,
wherein the logic-gated protein actuator system is a first-surface-antigen-AND-second-surface-antigen-NOT-third-surface-antigen-NOT-fourth-surface-antigen logic gate.

7. The logic-gated protein actuator system of claim 6,
wherein the mixture comprises at least one additional component comprising the second caged split intcin fragment fused to the modified second protein-of-interest fragment and fused to an additional antigen-binding domain configured to bind to an additional surface antigen,
wherein the additional surface antigen is different from the first surface antigen, from the second surface antigen, from the third surface antigen, and from the fourth surface antigen,
wherein the first caged split intein fragment and the second caged split intein fragment of the additional component are capable of undergoing protein trans-splicing to form the defective protein of interest from the first protein-of-interest fragment and the modified second protein-of-interest fragment when the additional antigen-binding domain and the first antigen-binding domain are proximal,
wherein the logic-gated protein actuator system is a first-surface-antigen-AND-second-surface-antigen-NOT-third-surface-antigen-NOT-fourth-surface-antigen-NOT-additional-surface-antigen logic gate.

8. The logic-gated protein actuator system of claim 5,
wherein the first surface antigen and the second surface antigen are identical and
wherein the logic-gated protein actuator system is a first-surface-antigen-NOT-third-surface-antigen logic gate.

9. The logic-gated protein actuator system of claim 6,
wherein the first surface antigen and the second surface antigen are identical and
wherein the logic-gated protein actuator system is a first-surface-antigen-AND-[third-surface-antigen-NOR-fourth-surface-antigen] logic gate.

10. The logic-gated protein actuator system of claim 1,
wherein the first surface antigen and the second surface antigen are different,
wherein the mixture comprises a third component comprising the first caged split intein fragment fused to the first protein-of-interest fragment and fused to the second antigen-binding domain,
wherein the mixture comprises a fourth component comprising the second caged split intcin fragment fused to the second protein-of-interest fragment and fused to the first antigen-binding domain,
wherein the first caged split intein fragment and the second caged split intein fragment are capable of undergoing protein trans-splicing to reconstitute the protein of interest from the first protein-of-interest fragment and the second protein-of-interest fragment when two first antigen-binding domains are proximal,
wherein the first caged split intein fragment and the second caged split intein fragment are capable of undergoing protein trans-splicing to reconstitute the protein of interest from the first protein-of-interest fragment and the second protein-of-interest fragment when two second antigen-binding domains are proximal, and
wherein the logic-gated protein actuator system is a first-surface-antigen-OR-second-surface-antigen logic gate.

11. The logic-gated protein actuator system of claim 10,
wherein the mixture comprises a fifth component comprising the first caged split intein fragment fused to the first protein-of-interest fragment and fused to a third antigen-binding domain,
wherein the mixture comprises a sixth component comprising the second caged split intein fragment fused to the second protein-of-interest fragment and fused to a fourth antigen-binding domain,
wherein the third antigen-binding domain is different from the second antigen-binding domain and from the first antigen-binding domain,
wherein the first caged split intein fragment and the second caged split intein fragment are capable of undergoing protein trans-splicing to reconstitute the protein of interest from the first protein-of-interest fragment and the second protein-of-interest fragment when the third antigen-binding domain is proximal to the first antigen-binding domain or the second antigen-binding domain or two third antigen-binding domains are proximal . . . and
wherein the logic-gated protein actuator system is a first-surface-antigen-OR-second-surface-antigen-OR-third-surface-antigen logic gate.

12. The logic-gated protein actuator system of claim 11,
wherein the mixture comprises at least one additional pair of components,
wherein the first part of the additional pair of components comprises the first caged split intein fragment fused to the first protein-of-interest fragment and fused to an additional antigen-binding domain configured to bind to an additional surface antigen,
wherein the second part of the additional pair of components comprises the second caged split intein fragment fused to the second protein-of-interest fragment and fused to the additional antigen-binding domain,
wherein each additional surface antigen is different from the first surface antigen, the second surface antigen, the third surface antigen, and each other additional surface antigen,
wherein the first caged split intein fragment and the second caged split intein fragment are capable of undergoing protein trans-splicing to form the protein of interest from the first protein-of-interest fragment and the second protein-of-interest fragment when the additional antigen-binding domain is proximal to the third antigen-binding domain, the second antigen-binding domain, the first antigen-binding domain, or any additional binding domain, and
wherein the logic-gated protein actuator system is a first-surface-antigen-OR-second-surface-antigen-OR-third-surface-antigen-OR-additional-surface-antigen(s) logic gate.

13. The logic-gated protein actuator system of claim 1,
wherein the first caged split intein fragment is eNrdJ-1N^{cage}-K114AK116A and
wherein the second caged split intein fragment is eNrdJ-1C^{cage}.

14. The logic-gated protein actuator system of claim 1,
wherein the first caged split intein fragment is eNrdJ-1C^{cage} and
wherein the second caged split intein fragment is eNrdJ-1N^{cage}-K114AK116A.

15. The logic-gated protein actuator system of claim 1,
wherein the pair of the first caged split intein fragment and the second caged split intein fragment are selected from the group of pairs consisting of eNrdJ-1N^{cage} & eNrdJ-1C^{cage}, NrdJ-IN^{cage} & NrdJ-1C^{cage}, NpUN^{cage} & NpuC^{cage}, Gp41-1N^{cage} & Gp41-1C^{cage}, Gp41-8N^{cage} & Gp41-80^{cage}, eNrdJ-1C^{cage} & eNrdJ-1N^{cage}, NrdJ-1C^{cage} & NrdJ-1N^{cage}, NpuC^{cage} & NpuN^{cage}, Gp41-1C^{cage} & Gp41-1N^{cage}, and Gp41-8C^{cage} & Gp41-8N^{cage}.

16. The logic-gated protein actuator system of claim 1,
wherein the protein of interest comprises a toxin.

17. The logic-gated protein actuator system of claim 1,
wherein the protein of interest comprises an imaging agent.

18. The logic-gated protein actuator system of claim 1,
wherein the protein of interest comprises an antibody epitope.

19. The logic-gated protein actuator system of claim 1,
wherein the protein of interest comprises an antigen or antigen fragment to which an antibody or an antibody paratope or a T-cell or a T-cell receptor can bind or a protein, protein fragment, antigen, antibody, or antigen-antibody complex to which a complement protein can bind.

20. The logic-gated protein actuator system of claim 1,
wherein the protein of interest comprises an enzyme.

21. The logic-gated protein actuator system of claim 1,
wherein the protein of interest is capable of binding a toxin.

22. The logic-gated protein actuator system of claim 1,
wherein the protein of interest is capable of binding an imaging agent.

23. The logic-gated protein actuator system of claim 1,
wherein the protein of interest is capable of binding an antibody epitope.

24. The logic-gated protein actuator system of claim 1,
wherein the protein of interest is capable of binding an antigen or an antigen fragment to which an antibody or an antibody paratope or a T-cell or a T-cell receptor can bind or a protein, protein fragment, antigen, antibody, or antigen-antibody complex to which a complement protein can bind.

25. The logic-gated protein actuator system of claim 1,
wherein the protein of interest is capable of binding an enzyme.

26. The logic-gated protein actuator system of claim 1,
wherein the protein of interest is SpyCatcher003.

27. The logic-gated protein actuator system of claim 1,
wherein the first protein of interest fragment is SpyN¹⁻⁷³ and
wherein the second protein of interest fragment is SpyC^{74-113.}

28. The logic-gated protein actuator system of claim 1,
wherein the first protein of interest fragment is SpyC⁷⁴⁻¹¹³ and
wherein the second protein of interest fragment is SpyN^{1-73.}

29. The logic-gated protein actuator system of claim 1,
wherein the pair of the first protein-of-interest fragment and the second protein-of-interest fragment are selected from the group of pairs consisting of SpyN¹⁻²⁴ & SpyC²⁵⁻¹¹³, SpyN¹⁻⁴² & SpyC⁴³⁻¹¹³, SpyN¹⁻⁵⁵ & SpyC⁵⁶⁻¹¹³, SpyN¹⁻⁸² & SpyC⁸³⁻¹¹³, SpyN¹⁻⁹⁰ & SpyC⁹¹⁻¹¹³, SpyC²⁵⁻¹¹³ & SpyN¹⁻²⁴, SpyC⁴³⁻¹¹³ & SpyN¹⁻⁴², SpyC⁵⁶⁻¹¹³ & SpyN¹⁻⁵⁵, SpyC⁸³⁻¹¹³ & SpyN¹⁻⁸², and SpyC⁹¹⁻¹¹³ & SpyN¹⁻⁹⁰.

30. The logic-gated protein actuator system of claim 1,
wherein the mixture further comprises a SpyTag003-biotin conjugate.

31. The logic-gated protein actuator system of claim 1,
wherein the mixture further comprises a SpyTag003-biotin conjugate and a Streptavidin-Saporin conjugate.

32. The logic-gated protein actuator system of claim 1,
wherein the mixture further comprises a SpyTag003-AF594 conjugate.

33. The logic-gated protein actuator system of claim 1,
wherein the mixture further comprises a SpyTag003-APEX2 conjugate.

34. The logic-gated protein actuator system of claim 1,
wherein the first surface antigen is selected from the group consisting of HER2, EGFR, and EpCAM and
wherein the second surface antigen is selected from the group consisting of HER2, EGFR, and EpCAM.

35. The logic-gated protein actuator system of claim 3,
wherein the first surface antigen is selected from the group consisting of HER2, EGFR, and EpCAM and
wherein the second surface antigen is selected from the group consisting of HER2, EGFR, and EpCAM,
wherein the third surface antigen is selected from the group consisting of HER2, EGFR, and EpCAM.

36. The logic-gated protein actuator system of claim 4,
wherein each surface antigen is selected from the group consisting of HER2, EGFR, and EpCAM.

37. A method of conditionally forming a protein of interest, comprising:
providing a mixture comprising
a first component comprising a first caged split intein fragment fused to a first protein-of-interest fragment and fused to a first antigen-binding domain configured to bind to a first surface antigen; and
a second component comprising a second caged split intein fragment fused to a second protein-of-interest fragment and fused to a second antigen-binding domain configured to bind to a second surface antigen,
bringing the mixture into contact with a cell having a surface, and
when the first surface antigen and the second surface antigen are proximal on the surface of the cell, allowing the first antigen-binding domain to bind to the first surface antigen and the second antigen-binding domain to bind to the second surface antigen, and allowing the first caged split intein fragment and the second caged split intein fragment to undergo protein trans-splicing to form a protein of interest from the first protein-of-interest fragment and the second protein-of-interest fragment,
wherein the first caged split intein fragment and the second caged split intein fragment are an actuator.

38. The method of claim 37, further comprising
allowing the protein of interest to bind a toxin, and
allowing the toxin to damage or kill the cell.

39. Treating a cancer by the method of claim 38,
further comprising administering the mixture and the toxin to a patient,
wherein the cell is a cancerous cell.

40. The method of claim 37, further comprising
allowing the protein of interest to bind an imaging agent, and
using the imaging agent to image the cell.

41. Diagnosing a hyperproliferative disorder by the method of claim 40,
further comprising administering the mixture and the imaging agent to a patient,
wherein the cell is a hyperproliferative cell.

42. The method of claim 37,
wherein the protein of interest comprises a toxin and
further comprising allowing the toxin to damage or kill the cell.

43. The method of claim 37,
wherein the protein of interest comprises an imaging agent and
further comprising using the imaging agent to image the cell.
